SEQUENCE LISTING (110 sequences)

SEQ ID NO: 1 - protein, 217 aa, Homo sapiens
  REGION 1..217: note = MISC_FEATURE - CH2-CH3 Domain of Exemplary Human IgG1
  VARIANT 217: note = X is lysine (K) or is absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 2 - protein, 216 aa, Homo sapiens
  REGION 1..216: note = MISC_FEATURE - CH2-CH3 Domain of Exemplary Human IgG2
  VARIANT 216: note = X is lysine (K) or is absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 3 - protein, 217 aa, Homo sapiens
  REGION 1..217: note = MISC_FEATURE - CH2-CH3 Domain of Exemplary Human IgG3
  VARIANT 217: note = X is lysine (K) or is absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 4 - protein, 217 aa, Homo sapiens
  REGION 1..217: note = MISC_FEATURE - CH2-CH3 Domain of Exemplary Human IgG4
  VARIANT 217: note = X is lysine (K) or is absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 5 - protein, 8 aa, synthetic construct
  REGION 1..8: note = Intervening Spacer Peptide (Linker 1)
GGGSGGGG

SEQ ID NO: 6 - protein, 5 aa, synthetic construct
  REGION 1..5: note = Cysteine-Containing Spacer Peptide (Linker 2)
GGCGG

SEQ ID NO: 7 - protein, 4 aa, synthetic construct
  REGION 1..4: note = Alternativel Spacer Peptide Linker 2
GGGS

SEQ ID NO: 8 - protein, 6 aa, synthetic construct
  REGION 1..6: note = Alternativel Spacer Peptide Linker 2
LGGGSG

SEQ ID NO: 9 - protein, 11 aa, synthetic construct
  REGION 1..11: note = Alternative Spacer Peptide Linker 2
GGGSGGGSGGG

SEQ ID NO: 10 - protein, 5 aa, synthetic construct
  REGION 1..5: note = Alternative Spacer Peptide Linker 2
ASTKG

SEQ ID NO: 11 - protein, 6 aa, synthetic construct
  REGION 1..6: note = Alternatice Spacer Pepptide Linker 2
LEPKSS

SEQ ID NO: 12 - protein, 5 aa, synthetic construct
  REGION 1..5: note = Alternative Spacer Peptide Linker 2
APSSS

SEQ ID NO: 13 - protein, 7 aa, synthetic construct
  REGION 1..7: note = Heterodimer-Promoting Domain
GVEPKSC

SEQ ID NO: 14 - protein, 6 aa, Homo sapiens
  REGION 1..6: note = MISC_FEATURE - Hinge Region of Human IgG Heterodimer-Promoting Domain
VEPKSC

SEQ ID NO: 15 - protein, 6 aa, Homo sapiens
  REGION 1..6: note = MISC_FEATURE - Hinge Region of Human IgG Heterodimer-Promoting Domain
AEPKSC

SEQ ID NO: 16 - protein, 6 aa, Homo sapiens
  REGION 1..6: note = MISC_FEATURE - Human Kappa Light Chain Heterodimer-Promoting Domain
GFNGEC

SEQ ID NO: 17 - protein, 6 aa, Homo sapiens
  REGION 1..6: note = MISC_FEATURE - Human Kappa Light Chain Heterodimer-Promoting Domain
FNRGEC

SEQ ID NO: 18 - protein, 28 aa, synthetic construct
  REGION 1..28: note = E-coil Heterodimer-Promoting Domain
EVAALEKEVAALEKEVAALEKEVAALEK

SEQ ID NO: 19 - protein, 28 aa, synthetic construct
  REGION 1..28: note = K-coil Heterodimer-Promoting Domain
KVAALKEKVAALKEKVAALKEKVAALKE

SEQ ID NO: 20 - protein, 28 aa, synthetic construct
  REGION 1..28: note = Cysteine-Containing E-coil Heterodimer-Promoting Domain
EVAACEKEVAALEKEVAALEKEVAALEK

SEQ ID NO: 21 - protein, 28 aa, synthetic construct
  REGION 1..28: note = Cysteine-Containing K-coil Heterodimer-Promoting Domain
KVAACKEKVAALKEKVAALKEKVAALKE

SEQ ID NO: 22 - protein, 46 aa, Streptococcus dysgalactiae
  REGION 1..46: note = MISC_FEATURE - Albumin-Binding Domain 3 (ABD3) of protein G of Streptococcus strain G148
LAEAKVLANRELDKYGVSDYYKNLIDNAKSAEGVKALIDEILAALP

SEQ ID NO: 23 - protein, 46 aa, synthetic construct; identical to SEQ ID NO: 22
  REGION 1..46: note = Deimmunized Variant of Albumin-Binding Domain 3 (ABD3) of protein G of Streptococcus strain G148

SEQ ID NO: 24 - protein, 46 aa, synthetic construct
  REGION 1..46: note = Deimmunized Variant of Albumin-Binding Domain 3 (ABD3) of protein G of Streptococcus strain G148
LAEAKVLANRELDKYGVSDYYKNAANNAKTVEGVKALIAEILAALP

SEQ ID NO: 25 - protein, 46 aa, synthetic construct
  REGION 1..46: note = Deimmunized Variant of Albumin-Binding Domain 3 (ABD3) of protein G of Streptococcus strain G148
LAEAKVLANRELDKYGVSDYYKNLISNAKSVEGVKALIAEILAALP

SEQ ID NO: 26 - protein, 10 aa, synthetic construct
  REGION 1..10: note = Intervening Spacer Linker
DKTHTCPPCP

SEQ ID NO: 27 - protein, 15 aa, synthetic construct
  REGION 1..15: note = Intervening Spacer Linker
EPKSCDKTHTCPPCP

SEQ ID NO: 28 - protein, 5 aa, synthetic construct
  REGION 1..5: note = Intervening Spacer Linker
APSSS

SEQ ID NO: 29 - protein, 8 aa, synthetic construct
  REGION 1..8: note = Intervening Spacer Linker
APSSSPME

SEQ ID NO: 30 - protein, 15 aa, synthetic construct
  REGION 1..15: note = Intervening Spacer Linker
LEPKSADKTHTCPPC

SEQ ID NO: 31 - protein, 13 aa, synthetic construct
  REGION 1..13: note = Intervening Spacer Linker
GGGDKTHTCPPCP

SEQ ID NO: 32 - protein, 16 aa, synthetic construct
  REGION 1..16: note = Intervening Spacer Linker
LEPKSSDKTHTCPPCP

SEQ ID NO: 33 - protein, 15 aa, Homo sapiens
  REGION 1..15: note = MISC_FEATURE - Exemplary IgG1 Hinge Region
EPKSCDKTHTCPPCP

SEQ ID NO: 34 - protein, 12 aa, Homo sapiens
  REGION 1..12: note = MISC_FEATURE - Exemplary IgG2 Hinge Region
ERKCCVECPPCP

SEQ ID NO: 35 - protein, 12 aa, Homo sapiens
  REGION 1..12: note = MISC_FEATURE - Exemplary IgG4 Hinge Region
ESKYGPPCPSCP

SEQ ID NO: 36 - protein, 12 aa, synthetic construct
  REGION 1..12: note = IgG4 Hinge Variant comprising Stabilizing S228P Substitution
ESKYGPPCPPCP

SEQ ID NO: 37 - protein, 15 aa, synthetic construct
  REGION 1..15: note = Intervening Spacer Peptide
GGGGSGGGGSGGGGS

SEQ ID NO: 38 - protein, 107 aa, Homo sapiens [sequence omitted]

SEQ ID NO: 39 - protein, 104 aa, Homo sapiens
  REGION 1..104: note = MISC_FEATURE - Exemplary Human IgG CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 40 - protein, 98 aa, Homo sapiens
  REGION 1..98: note = MISC_FEATURE - Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 41 - protein, 98 aa, Homo sapiens
  REGION 1..98: note = MISC_FEATURE - Human IgG2 CH1 Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 42 - protein, 98 aa, Homo sapiens
  REGION 1..98: note = MISC_FEATURE - Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 43 - protein, 217 aa, synthetic construct
  REGION 1..217: note = CH2-CH3 Domains of IgG1 Comprising Substitutions L234A and L235A (Kabat)
  VARIANT 217: note = X is lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 44 - protein, 217 aa, synthetic construct
  REGION 1..217: note = "Knob-Bearing" IgG1 CH2-CH3 Domain
  VARIANT 217: note = X is lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 45 - protein, 217 aa, synthetic construct
  REGION 1..217: note = "Hole-Bearing" IgG1 CH2-CH3 Domain
  VARIANT 217: note = X is lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 46 - protein, 288 aa, Homo sapiens
  REGION 1..288: note = MISC_FEATURE - Human PD-1, Including Signal Sequence (NCBI Sequence NP_005009.2)
  SIGNAL 1..20: note = Signal Sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 47 - protein, 113 aa, Homo sapiens [sequence omitted]

SEQ ID NO: 48 - protein, 107 aa, Homo sapiens [sequence omitted]

SEQ ID NO: 49 - protein, 120 aa, synthetic construct [sequence omitted]

SEQ ID NO: 50 - protein, 111 aa, synthetic construct [sequence omitted]

SEQ ID NO: 51 - protein, 121 aa, Mus musculus
  REGION 1..121: note = MISC_FEATURE - VH Domain of Murine Anti-Human PD-1 Antibody PD-1 mAb 3 (EH12.2H7)
QVQLQQSGAELAKPGASVQMSCKASGYSFTSSWIHWVKQRPGQGLEWIGYIYPSTGFTEYNQKFKDKATLTADKSSSTAYMQLSSLTSEDSAVYYCARWRDSSGYHAMDYWGQGTSVTVSS

SEQ ID NO: 52 - protein, 111 aa, Mus musculus
  REGION 1..111: note = MISC_FEATURE - VL Domain of Murine Anti-Human PD-1 Antibody PD-1 mAb 3 (EH12.2H7)
DIVLTQSPASLTVSLGQRATISCRASQSVSTSGYSYMHWYQQKPGQPPKLLIKFGSNLESGIPARFSGSGSGTDFTLNIHPVEEEDTATYYCQHSWEIPYTFGGGTKLEIK

SEQ ID NO: 53 - protein, 117 aa, synthetic construct
  REGION 1..117: note = VH Domain of Humanized Anti-Human Antibody PD-1 mAb 4 (Pidilizumab)
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 54 - protein, 106 aa, synthetic construct
  REGION 1..106: note = VL Domain of Humanized Anti-Human Antibody PD-1 mAb 4 (Pidilizumab)
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 55 - protein, 116 aa, synthetic construct
  REGION 1..116: note = VH Domain of Humanized Anti-Human PD-1 Antibody (PD-1 mAb 5)
EVQLVESGGGLVQPGGSLRLSCAASGFVFSSFGMHWVRQAPGKGLEWVAYISSGSMSISYADTVKGRFTISRDNAKNTLYLQMNSLRTEDTALYYCASLSDYFDYWGQGTTVTVSS

SEQ ID NO: 56 - protein, 112 aa, synthetic construct
  REGION 1..112: note = VL Domain of Humanized Anti-Human PD-1 Antibody (PD-1 mAb 5)
DVVMTQSPLSLPVTLGQPASISCRSSQSLVHSTGNTYLHWYLQKPGQSPQLLIYRVSNRFSGVPDRFSGSGSGTDFTLKISRVEAEDVGVYYCSQTTHVPWTFGQGTKLEIK

SEQ ID NO: 57 - protein, 119 aa, synthetic construct
  REGION 1..119: note = VH Domain of Humnaized Anti-Human PD-1 Antibody (PD-1 mAb 6)
  VARIANT 48: note = X is isoleucine (I) or alanine (A)
QVQLVQSGAEVKKPGASVKVSCKASGYSFTSYWMNWVRQAPGQGLEWXGVIHPSDSETWLDQKFKDRVTITVDKSTSTAYMELSSLRSEDTAVYYCAREHYGTSPFAYWGQGTLVTVSS

SEQ ID NO: 58 - protein, 111 aa, synthetic construct
  REGION 1..111: note = VL Domain of Humanized Anti-Human PD-1 Antibody (PD-1 mAb 6)
  VARIANT 26: note = X is asparagine (N) or serine (S)
  VARIANT 58: note = X is glutamine (Q) or arginine (R)
EIVLTQSPATLSLSPGERATLSCRAXESVDNYGMSFMNWFQQKPGQPPKLLIHAASNXGSGVPSRFSGSGSGTDFTLTISSLEPEDFAVYFCQQSKEVPYTFGGGTKVEIK

SEQ ID NO: 59 - protein, 119 aa, synthetic construct
  REGION 1..119: note = VH Domain of Humanized Anti-Human PD-1 Antibody (PD-1 mAb 7)
  VARIANT 12: note = X is valine (V) or alanine (A)
  VARIANT 35: note = X is serine (S) or glycine (G)
  VARIANT 48: note = X is valine (V) or threonine (T)
  VARIANT 86: note = X is leucine (L) or alanine (A)
EVQLVESGGGLXRPGGSLKLSCAASGFTFSSYLVXWVRQAPGKGLEWXATISGGGGNTYYSDSVKGRFTISRDNAKNSLYLQMNSXRAEDTATYYCARYGFDGAWFAYWGQGTLVTVSS

SEQ ID NO: 60 - protein, 107 aa, synthetic construct
  REGION 1..107: note = VL Domain of Humanized Anti-Human PD-1 Antibody (PD-1 mAb 7)
  VARIANT 31: note = X is serine (S) or asparagine (N)
  VARIANT 50: note = X is asparagine (N) or asparatate (D)
DIQMTQSPSSLSASVGDRVTITCRASENIYXYLAWYQQKPGKAPKLLIYXAKTLAAGVPSRFSGSGSGTDFTLTISSLQPEDFATYYCQHHYAVPWTFGQGTKLEIK

SEQ ID NO: 61 - protein, 117 aa, synthetic construct
  REGION 1..117: note = VH Domain of Humanized Anti-Human PD-1 Antibody (PD-1 mAb 8)
EVQLVESGGGLVRPGGSLRLSCAASGFTFSSYLISWVRQAPGKGLEWVAAISGGGADTYYADSVKGRFTISRDNAKNSLYLQMNSLRAEDTATYYCARRGTYAMDYWGQGTLVTVSS

SEQ ID NO: 62 - protein, 107 aa, synthetic construct
  REGION 1..107: note = VL Domain of Humanized Anti-Human PD-1 Antibody (PD-1 mAb 8)
DIQMTQSPSSLSASVGDRVTITCRASENIYNYLAWYQQKPGKAPKLLIYDAKTLAAGVPSRFSGSGSGTDFTLTISSLQPEDFATYYCQHHYAVPWTFGQGTKLEIK

SEQ ID NO: 63 - protein, 496 aa, synthetic construct
  REGION 1..496: note = First and Third Polypeptide Chains of PD-1 x LAG-3 DART A
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 64 - protein, 271 aa, synthetic construct
  REGION 1..271: note = Second and Fourth Polypeptide Chains of PD-1 x LAG-3 DART A
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 65 - protein, 118 aa, Mus musculus
  REGION 1..118: note = MISC_FEATURE - VH Domain of mAb 4-4-20
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 66 - protein, 112 aa, Mus musculus
  REGION 1..112: note = MISC_FEATURE - VL Domain of mAb 4-4-20
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIK

SEQ ID NO: 67 - protein, 273 aa, synthetic construct
  REGION 1..273: note = First Polypeptide Chain of Universal Bispecific Adaptor Molecule UBA-1
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAALEKEVAALEKEVAALEKEVAALEK

SEQ ID NO: 68 - protein, 272 aa, synthetic construct
  REGION 1..272: note = Second Polypeptide Chain of Universal Bispecific Adaptor Molecule UBA-1
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGGSGGGGEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSSGGCGGGKVAALKEKVAALKEKVAALKEKVAALKE

SEQ ID NO: 69 - protein, 503 aa, synthetic construct
  REGION 1..503: note = First and Third Polypeptides of Universal Bispecific Adaptor Molecule UBA-2
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 503 aa, synthetic construct
  REGION 1..503: note = First Polypeptide Chain of Universal Bispecific Adaptor Molecule UBA-3
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 227 aa, synthetic construct
  REGION 1..227: note = Third Polypeptide Chain of Universal Bispecific Adaptor Molecule UBA-3
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 72 - protein, 449 aa, synthetic construct
  REGION 1..449: note = Third Polypeptide Chain of Universal Bispecific Adaptor Molecule UBA-4
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 73 - protein, 218 aa, synthetic construct
  REGION 1..218: note = Fourth Polypeptide Chain of Universal Bispecific Adaptor Molecule UBA-4
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 575 aa, synthetic construct
  REGION 1..575: note = Third Polypeptide Chain of Universal Bispecific Adaptor Molecule UBA-5
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGGSGGGGSGGGGSQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 75 - protein, 223 aa, Homo sapiens
  REGION 1..223: note = MISC_FEATURE - Human CTLA-4, Including Signal Sequence (NCBI Sequence NP_005205.2)
  SIGNAL 1..34: note = Signal Sequence
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 76 - protein, 118 aa, Homo sapiens
  REGION 1..118: note = MISC_FEATURE - VH Domain of Anti-Human CTLA-4 Antibody CTLA-4 mAb 1 (Ipilimumab)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 77 - protein, 108 aa, Homo sapiens
  REGION 1..108: note = MISC_FEATURE - VL Domain of Anti-Human CTLA-4 Antibody CTLA-4 mAb 1 (Ipilimumab)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 78 - protein, 125 aa, Homo sapiens
  REGION 1..125: note = MISC_FEATURE - VH Domain of Anti-Human CTLA-4 Antibody CTLA-4 mAb 2 (Tremelimumab)
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 79 - protein, 107 aa, Homo sapiens
  REGION 1..107: note = MISC_FEATURE - VL Domain of Anti-Human CTLA-4 Antibody CTLA-4 mAb 2 (Tremelimumab)
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 80 - protein, 217 aa, synthetic construct
  REGION 1..217: note = CH2-CH3 Domains of IgG1 Fc Region-Comprising Substitutions M252Y, S254T and T256E
  VARIANT 217: note = X is lysine (K) or is absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 81 - protein, 217 aa, synthetic construct
  REGION 1..217: note = CH2-CH3 Domains of IgG4 Fc Region-Comprising Substitutions M252Y, S254T and T256E
  VARIANT 217: note = X is lysine (K) or is absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 82 - protein, 217 aa, synthetic construct
  REGION 1..217: note = "Knob-Bearing" Variant of CH2-CH3 Domains of IgG1 Fc Region-Comprising Substitutions M252Y, S254T and T256E
  VARIANT 217: note = X is lysine (K) or is absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 83 - protein, 217 aa, synthetic construct
  REGION 1..217: note = "Hole-Bearing" Variant of CH2-CH3 Domains of IgG1 Fc Region-Comprising Substitutions M252Y, S254T and T256E
  VARIANT 217: note = X is lysine (K) or is absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 84 - protein, 217 aa, synthetic construct
  REGION 1..217: note = "Knob-Bearing" Variant of CH2-CH3 Domains of IgG4 Fc Region-Comprising Substitutions M252Y, S254T and T256E
  VARIANT 217: note = X is lysine (K) or is absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGX

SEQ ID NO: 85 - protein, 217 aa, synthetic construct
  REGION 1..217: note = "Hole-Bearing" Variant of CH2-CH3 Domains of IgG4 Fc Region-Comprising Substitutions M252Y, S254T and T256E
  VARIANT 217: note = X is lysine (K) or is absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGX

SEQ ID NO: 86 - protein, 119 aa, synthetic construct
  REGION 1..119: note = VH Domain of Humanized Anti-Human PD-1 Antibody (PD-1 mAb 6 I VH)
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 87 - protein, 111 aa, synthetic construct
  REGION 1..111: note = VL Domain of Humanized Anti-Human PD-1 Antibody (PD-1 mAb 6 SQ VL)
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 88 - protein, 445 aa, synthetic construct
  REGION 1..445: note = Heavy Chain of Anti-Human PD-1 Antibody PD-1 mAb 6 G4P
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 89 - protein, 218 aa, synthetic construct; identical to SEQ ID NO: 73
  REGION 1..218: note = Light Chain of Anti-Human PD-1 Antibody PD-1 mAb 6 G4P

SEQ ID NO: 90 - protein, 118 aa, Mus musculus
  REGION 1..118: note = MISC_FEATURE - VH Domain of Anti-Human CTLA-4 Antibody CTLA-4 mAb 3
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSS

SEQ ID NO: 91 - protein, 108 aa, Mus musculus
  REGION 1..108: note = MISC_FEATURE - VL Domain of Anti-Human CTLA-4 Antibody CTLA-4 mAb 3
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIK

SEQ ID NO: 92 - protein, 448 aa, Mus musculus
  REGION 1..448: note = MISC_FEATURE - Heavy Chain of Anti-Human CTLA-4 Antibody mAb 3 G1AA
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 444 aa, Mus musculus
  REGION 1..444: note = MISC_FEATURE - Heavy Chain of Anti-Human CTLA-4 Antibody mAb 3 G4P
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 94 - protein, 215 aa, Mus musculus
  REGION 1..215: note = MISC_FEATURE - Light Chain of Anti-Human CTLA-4 Antibodies mAb 3 G1AA and mAb 3 G4P
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 497 aa, synthetic construct
  REGION 1..497: note = First and Third Polypeptide Chains of DART B
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 96 - protein, 271 aa, synthetic construct
  REGION 1..271: note = Second and Fourth Polypeptide Chains of DART B
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGCGGGKVAACKEKVAALKEKVAALKEKVAALKE

SEQ ID NO: 97 - protein, 497 aa, synthetic construct
  REGION 1..497: note = First and Third Polypeptide Chains of DART C
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 98 - protein, 271 aa, synthetic construct
  REGION 1..271: note = Second and Fourth Polypeptide Chains of DART C
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSGGCGGGKVAACKEKVAALKEKVAALKEKVAALKE

SEQ ID NO: 99 - protein, 499 aa, synthetic construct
  REGION 1..499: note = First and Third Polypeptide Chains of DART D
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 269 aa, synthetic construct
  REGION 1..269: note = Second and Fourth Polypeptide Chains of DART D
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGKVAACKEKVAALKEKVAALKEKVAALKE

SEQ ID NO: 101 - protein, 503 aa, synthetic construct
  REGION 1..503: note = First and Third Polypeptide Chains of DART F
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSGGCGGGEVAACEKEVAALEKEVAALEKEVAALEKLEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 567 aa, synthetic construct
  REGION 1..567: note = First and Third Polypeptide Chains of DART E
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 103 - protein, 350 aa, synthetic construct
  REGION 1..350: note = Second and Fourth Polypeptide Chains of DART E
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSLGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 500 aa, synthetic construct
  REGION 1..500: note = First Polypeptide Chain of TRIDENT A
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 105 - protein, 272 aa, synthetic construct
  REGION 1..272: note = Second Polypeptide Chain of TRIDENT A
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGKVAACKEKVAALKEKVAALKEKVAALKE

SEQ ID NO: 106 - protein, 444 aa, synthetic construct
  REGION 1..444: note = Third Polypeptide Chain of TRIDENT A
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLG

SEQ ID NO: 107 - protein, 215 aa, synthetic construct; identical to SEQ ID NO: 94
  REGION 1..215: note = Fourth Polypeptide Chain of TRIDENT A

SEQ ID NO: 108 - protein, 502 aa, synthetic construct
  REGION 1..502: note = First Polypeptide Chain of TRIDENT B
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKGGGDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 448 aa, synthetic construct
  REGION 1..448: note = Third Polypeptide Chain of TRIDENT B
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 110 - protein, 469 aa, synthetic construct
  REGION 1..469: note = Third Polypeptide Chain of TRIDENT C
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLG

(19) United States
(12) Patent Application Publication
(10) Pub. No.: US 2024/0084015 A1
(43) Pub. Date: Mar. 14, 2024

(54) METHODS OF USING BISPECIFIC MOLECULES HAVING IMMUNOREACTIVITY WITH PD-1 AND CTLA-4

(71) Applicant: MACROGENICS, INC., Rockville, MD (US)

(72) Inventors: Leslie S. JOHNSON, Darnestown, MD (US); Gurunadh Reddy CHICHILI, Germantown, MD (US); Kalpana SHAH, Boyds, MD (US); Ross LA MOTTE-MOHS, Boyds, MD (US); Paul A. MOORE, North Potomac, MD (US); Ezio BONVINI, Potomac, MD (US); Scott KOENIG, Rockville, MD (US)

(21) Appl. No.: 18/499,658

(22) Filed: Nov. 1, 2023

Related U.S. Application Data
(63) Continuation of application No. 17/148,309, filed on Jan. 13, 2021, now Pat. No. 11,840,571.
Application No. 17/148,309 is a division of application No. 16/060,227, filed on Jun. 7, 2018, now Pat. No. 10,954,301.
(60) Provisional application No. 62/266,944, filed on Dec. 14, 2015.

(51) Int. Cl. C07K 16/28; A61K 39/395; A61P 31/00; A61P 35/00; C07K 16/30; C07K 16/44; C07K 16/46

(52) U.S. Cl. CPC C07K 16/2827; A61K 39/395; A61P 31/00; A61P 35/00; C07K 16/28; C07K 16/2803; C07K 16/2818; C07K 16/30; C07K 16/44; C07K 16/46; C07K 16/468; A61K 38/00; C07K 2317/31; C07K 2317/35; C07K 2317/52; C07K 2317/53; C07K 2317/622; C07K 2317/626; C07K 2317/64; C07K 2317/76; C07K 2317/92; C07K 2317/94; C07K 2319/01

(57) ABSTRACT

The present invention is directed to bispecific molecules (e.g., diabodies, bispecific antibodies, trivalent binding molecules, etc.) that possess at least one epitope-binding site that is immunospecific for an epitope of PD-1 and at least one epitope-binding site that is immunospecific for an epitope of CTLA-4 (i.e., a “PD-1×CTLA-4 bispecific molecule”). The PD-1×CTLA-4 bispecific molecules of the present invention are capable of simultaneously binding to PD-1 and to CTLA-4, particularly as such molecules are arrayed on the surfaces of human cells. The invention is directed to pharmaceutical compositions that contain such PD-1×CTLA-4 bispecific molecules, and to methods involving the use of such bispecific molecules in the treatment of cancer and other diseases and conditions. The present invention also pertains to methods of using such PD-1×CTLA-4 bispecific molecules to stimulate an immune response.

### CROSS-REFERENCE TO RELATED APPLICATIONS

This patent application is a divisional application of U.S. patent application Ser. No. 16/060,227 filed on Jun. 7, 2018 (allowed), which is a 35 U.S.C. 371 national stage patent application of Patent Cooperation Treaty Patent Application No. PCT/US2016/066060 filed on Dec. 12, 2016 (expired), which claims the benefit of U.S. Patent Application No. 62/266,944 filed on Dec. 14, 2015 (expired). Each of the foregoing patent applications is incorporated herein by reference in its entirety.

### REFERENCE TO SEQUENCE LISTING

The instant application contains a Sequence Listing (0260-0003US3_SL.xml; size 169,737 bytes; and Date of Creation Nov. 1, 2023) which has been submitted electronically and is hereby incorporated by reference in its entirety.

### FIELD OF THE INVENTION

The present invention is directed to bispecific molecules (e.g., diabodies, bispecific antibodies, trivalent binding molecules, etc.) that possess at least one epitope-binding site that is immunospecific for an epitope of PD-1 and at least one epitope-binding site that is immunospecific for an epitope of CTLA-4 (i.e., a “PD-1×CTLA-4 bispecific molecule”). The present invention concerns such PD-1×CTLA-4 bispecific molecules that possess two epitope-binding sites that are immunospecific for one (or two) epitope(s) of PD-1 and two epitope-binding sites that are immunospecific for one (or two) epitope(s) of CTLA-4. The present invention also is directed to such PD-1×CTLA-4 bispecific molecules that additionally comprise an immunoglobulin Fc Region. The PD-1×CTLA-4 bispecific molecules of the present invention are capable of simultaneously binding to PD-1 and to CTLA-4, particularly as such molecules are arrayed on the surfaces of human cells. The invention is directed to pharmaceutical compositions that contain such PD-1×CTLA-4 bispecific molecules, and to methods involving the use of such bispecific molecules in the treatment of cancer and other diseases and conditions. The present invention also pertains to methods of using such PD-1×CTLA-4 bispecific molecules to stimulate an immune response.

### BACKGROUND OF THE INVENTION

### I. The Immune System Response to Cancer

The mammalian immune system is naturally poised to recognize and eliminate cancerous cells (Topalian, S. L. et al. (2015) “Immune Checkpoint Blockade: A Common Denominator Approach to Cancer Therapy,” Cancer Cell 27:450-461). In healthy individuals, the immune system is in a quiescent state, inhibited by a repertoire of diverse inhibitory receptors and ligands. Such immune “checkpoint” pathways are important in maintaining self-tolerance (i.e., in preventing a subject from mounting an immune system attack against his/her own cells (an “autoimmune” reaction) and in limiting collateral tissue damage during anti-microbial or anti-allergic immune responses. Upon recognition of a cancer antigen, detection of a microbial pathogen, or the presence of an allergen, an array of activating receptors and ligands induce the activation of the immune system. Such activation leads to the activation of macrophages, Natural Killer (NK) cells and antigen-specific, cytotoxic, T-cells, and promotes the release of various cytokines, all of which act to counter the perceived threat to the health of the subject (Dong, C. et al. (2003) “Immune Regulation by Novel Costimulatory Molecules,” Immunolog. Res. 28(1):39-48; Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). The immune system is capable of returning to its normal quiescent state when the countervailing inhibitory immune signals outweigh the activating immune signals.

Thus, the disease state of cancer (and indeed the disease states of infectious diseases) may be considered to reflect a failure to adequately activate a subject's immune system. Such failure may reflect an inadequate presentation of activating immune signals, or it may reflect an inadequate ability to alleviate inhibitory immune signals in the subject. In some instances, researchers have determined that cancer cells can co-opt the immune system to evade being detected by the immune system (Topalian, S. L. et al. (2015) “Immune Checkpoint Blockade: A Common Denominator Approach to Cancer Therapy,” Cancer Cell 27:450-461).

Of particular importance is binding between the B7.1 (CD80) and B7.2 (CD86) ligands of the Antigen-Presenting Cell and the CD28 and CTLA-4 receptors of the CD4⁺ T lymphocyte (Sharpe, A. H. et al. (2002) “The B7-CD28 Superfamily,” Nature Rev. Immunol. 2:116-126; Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321). Binding of B7.1 or of B7.2 to CD28 stimulates T-cell activation; binding of B7.1 or B7.2 to CTLA-4 inhibits such activation (Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321; Greenwald, R. J. et al. (2005) “The B7 Family Revisited,” Ann. Rev. Immunol. 23:515-548). CD28 is constitutively expressed on the surface of T-cells (Gross, J., et al. (1992) “Identification And Distribution Of The Costimulatory Receptor CD28 In The Mouse,” J. Immunol. 149:380-388), whereas CTLA-4 expression is rapidly upregulated following T-cell activation (Linsley, P. et al. (1996) “Intracellular Trafficking Of CTLA4 And Focal Localization Towards Sites Of TCR Engagement,” Immunity 4:535-543). Since CTLA-4 is the higher affinity receptor (Sharpe, A. H. et al. (2002) “The B7-CD28 Superfamily,” Nature Rev. Immunol. 2:116-126; Topalian, S. L. et al. (2015) “Immune Checkpoint Blockade: A Common Denominator Approach to Cancer Therapy,” Cancer Cell 27:450-461), binding first initiates T-cell proliferation (via CD28) and then inhibits it (via nascent expression of CTLA-4), thereby dampening the effect when proliferation is no longer needed.

### II. CTLA-4

Cytotoxic T-lymphocyte associated protein-4 (CTLA-4; CD152) is a single pass type I membrane protein that forms a disulfide linked homo-dimer (Schwartz J. C., et al. (2001) “Structural Basis For Co-Stimulation By The Human CTLA-4 B7-2 Complex,” Nature 410:604-608). Alternate splice variants, encoding different isoforms, have been characterized including a soluble isoform which functions as a monomer (Magistrelli G., et al. (1999) “A Soluble Form Of CTLA-4 Generated By Alternative Splicing Is Expressed By Nonstimulated Human T Cells,” Eur. J. Immunol. 29:3596-3602; Oaks M. K. et al. (2000) “A Native Soluble Form Of CTLA-4,” Cell Immunol 201:144-153).

CTLA-4 is primarily an intracellular antigen whose surface expression is tightly regulated by restricted trafficking to the cell surface and rapid internalization (Alegre M-L, et al., (1996) “Regulation Of Surface And Intracellular Expression Of CTLA4 On Mouse T Cells,” J. Immunol. 157:4762-4770; Linsley, P. S. et al. (1996) “Intracellular Trafficking Of CTLA-4 And Focal Localization Towards Sites Of TCR Engagement,” Immunity 4:535-543). CTLA-4 is expressed at low levels on the surface of naïve effector T-cells (Alegre, M. L., et al. (1996) “Regulation Of Surface And Intracellular Expression Of CTLA4 On Mouse T Cells,” J Immunol 157:4762-70), and constitutively expressed on T regulatory cells (Wang, X. B., et al. (2002) “Expression Of CTLA-4 By Human Monocytes,” Scand. J. Immunol. 55:53-60).

The extracellular region of CTLA-4 comprises a single extracellular Ig(V) domain, followed by a transmembrane (TM) region and a small intracellular cytoplasmic tail (37 amino acids). The intracellular tail contains two tyrosine-based motifs, which interact with several intracellular proteins, including the lipid kinase phosphatidylinositol 3-kinase (PI3K), the phosphatases SHP-2 and PP2A and clathrin adaptor proteins AP-1 and AP-2 (Rudd, C. E. et al. (2003) “Unifying Concepts In CD28, ICOS And CTLA4 Co-Receptor Signalling,” Nat Rev Immunol. 3:544-56). CTLA-4 is related to CD28, with the two proteins having approximately 29% identity at the amino acid level (Harper, K. (1991) “CTLA-4 And CD28 Activated Lymphocyte Molecules Are Closely Related In Mouse And Human As To Sequence, Message Expression, Gene Structure, And Chromosomal Location,” J. Immunol. 147:1037-1044).

When a naïve T effector cell is activated through its T-cell receptors (TCRs), CTLA-4 is recruited to the cell surface (Linsley, P. S., et al. (1996) “Intracellular Trafficking Of CTLA-4 And Focal Localization Towards Sites Of TCR Engagement,” Immunity 4:535-43). Once CTLA-4 is expressed on the T-cell surface, it competes with CD28 (constitutively expressed on T-cells) for CD80/CD86, thereby shutting off further signaling through the TCR and thus down-regulating any further T-cell response by TCR signaling (Carreno, B. M., et al. (2000) “CTLA-4 (CD152) Can Inhibit T Cell Activation By Two Different Mechanisms Depending On Its Level Of Cell Surface Expression,” J Immunol 165:1352-6; Chuang, E., et al. (1999) “Regulation Of Cytotoxic T Lymphocyte-Associated Molecule-4 By Src Kinases,” J Immunol 162:1270-7). Thus, CTLA-4 acts as a negative regulator of T effector cell activation that diminishes effector function and dictates the efficacy and duration of a T-cell response (Linsley, P. S., et al. (1996) “Intracellular Trafficking Of CTLA-4 And Focal Localization Towards Sites Of TCR Engagement,” Immunity 4:535-43).

In addition, CTLA-4 may play a role in enhancing the negative effect of regulatory T-cells on the immune response to cancer (Tai, Y. T., et al., (2012) “Potent in vitro And in vivo Activity Of An Fc-Engineered Humanized Anti-HM1.24 Antibody Against Multiple Myeloma via Augmented Effector Function,” Blood 119:2074-82). CTLA-4 has a much higher affinity for members of the B7 family than for CD28, and therefore its expression on a T-cell dictates a dominant negative regulation of the T-cell (Allison, J. P., et al. (1995) “Manipulation Of Costimulatory Signals To Enhance Antitumor T-Cell Responses,” Curr Opin Immunol 7:682-6). The mechanism by which CTLA-4 contributes to the suppressor function of T regulatory cells is incompletely understood, but the expression of CTLA-4 on T regulatory cells enhances the suppressive function of these cells (Tai, Y. T., et al., (2012) “Potent in vitro And in vivo Activity Of An Fc-Engineered Humanized Anti-HM1.24 Antibody Against Multiple Myeloma via Augmented Effector Function,” Blood 119:2074-82).

Blockage of CTLA-4 is reported to enhance T-cell responses in vitro (Walunas, T. L., et al. (1994) “CTLA-4 Can Function As A Negative Regulator Of T Cell Activation,” Immunity 1:405-413) and in vivo (Kearney, E. R., et al. (1995) “Antigen-Dependent Clonal Expansion Of A Trace Population Of Antigen-Specific CD4+ T Cells in vivo Is Dependent On CD28 Costimulation And Inhibited By CTLA-4,” J. Immunol. 155:1032-1036) and also to increase antitumor immunity (Leach, D. R. et al. (1996) “Enhancement Of Antitumor Immunity By CTLA-4 Blockade,” Science 271:1734-1736). Thus, blockage of CTLA-4 using anti-CTLA-4 antibodies has been proposed to provide new treatments for disease, especially human diseases where immune stimulation might be beneficial such as for treatment of cancers and infectious diseases (see, Leach, D. R., et al. (1996) “Enhancement Of Antitumor Immunity By CTLA-4 Blockade,” Science. 271:1734-1736; and PCT Publications No. WO 01/14424; WO 00/37504). Development of blockers of CTLA-4 function has focused on the use of monoclonal antibodies such as ipilimumab (see, e.g., Hodi, F. S., et al., (2003) “Biologic Activity Of Cytotoxic T Lymphocyte-Associated Antigen 4 Antibody Blockade In Previously Vaccinated Metastatic Melanoma And Ovarian Carcinoma Patients,” Proc. Natl. Acad. Sci. (U.S.A.) 100:4717-4717) and tremelimumab (Ribas, A. et al. (2005) “Antitumor Activity In Melanoma And Anti-Self Responses In A Phase I Trial With The Anti-Cytotoxic T Lymphocyte-Associated Antigen 4 Monoclonal Antibody CP-675,206,” Oncologist 12: 873-883).

### III. Programmed Death-1 (“PD-1”)

Programmed Death-1 (“PD-1,” also known as “CD279”) is type I membrane protein member of the extended CD28/CTLA-4 family of T-cell regulators that broadly negatively regulates immune responses (Ishida, Y. et al. (1992) “Induced Expression Of PD-1, A Novel Member Of The Immunoglobulin Gene Superfamily, Upon Programmed Cell Death,” EMBO J. 11:3887-3895; United States Patent Application Publications No. 2007/0202100; 2008/0311117; 2009/00110667; U.S. Pat. Nos. 6,808,710; 7,101,550; 7,488,802; 7,635,757; 7,722,868; PCT Publication No. WO 01/14557).

The receptor-ligand interactions of the PD-1 system appear to be even more complex than those of the CD28/CTLA-4 system. PD-1 is expressed on the cell surface of activated T-cells, B-cells, and monocytes (Agata, Y. et al. (1996) “Expression Of The PD-1 Antigen On The Surface Of Stimulated Mouse T And B Lymphocytes,” Int. Immunol. 8(5):765-772; Yamazaki, T. et al. (2002) “Expression Of Programmed Death 1 Ligands By Murine T-Cells And APC,” J. Immunol. 169:5538-5545) and at low levels in natural killer (NK) T-cells (Nishimura, H. et al. (2000) “Facilitation Of Beta Selection And Modification Of Positive Selection In The Thymus Of PD-1-Deficient Mice,” J. Exp. Med. 191:891-898; Martin-Orozco, N. et al. (2007) “Inhibitory Costimulation And Anti-Tumor Immunity,” Semin. Cancer Biol. 17(4):288-298).

The extracellular region of PD-1 consists of a single immunoglobulin (Ig)V domain with 23% identity to the equivalent domain in CTLA-4 (Martin-Orozco, N. et al. (2007) “Inhibitory Costimulation And Anti-Tumor Immunity,” Semin. Cancer Biol. 17(4):288-298). The extracellular IgV domain is followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals (Ishida, Y. et al. (1992) “Induced Expression Of PD-1, A Novel Member Of The Immunoglobulin Gene Superfamily, Upon Programmed Cell Death,” EMBO J. 11:3887-3895; Blank, C. et al. (2006) “Contribution Of The PD-L1/PD-1 Pathway To T-Cell Exhaustion: An Update On Implications For Chronic Infections And Tumor Evasion Cancer,” Immunol. Immunother. 56(5):739-745).

PD-1 mediates its inhibition of the immune system by binding to B7-H1 and B7-DC (also known as PD-L1 and PD-L2, Flies, D. B. et al. (2007) “The New B7s: Playing a Pivotal Role in Tumor Immunity,” J. Immunother. 30(3):251-260; U.S. Pat. Nos. 6,803,192; 7,794,710; United States Patent Application Publication Nos. 2005/0059051; 2009/0055944; 2009/0274666; 2009/0313687; PCT Publication Nos. WO 01/39722; WO 02/086083).

B7-H1 and B7-DC are broadly expressed on the surfaces of many types of human and murine tissues, such as heart, placenta, muscle, fetal liver, spleen, lymph nodes, and thymus as well as murine liver, lung, kidney, islets cells of the pancreas and small intestine (Martin-Orozco, N. et al. (2007) “Inhibitory Costimulation And Anti-Tumor Immunity,” Semin. Cancer Biol. 17(4):288-298). In humans, B7-H1 protein expression has been found in human endothelial cells (Chen, Y. et al. (2005) “Expression of B7-H1 in Inflammatory Renal Tubular Epithelial Cells,” Nephron. Exp. Nephrol. 102:e81-e92; de Haij, S. et al. (2005) “Renal Tubular Epithelial Cells Modulate T-Cell Responses Via ICOS-L And B7-HT” Kidney Int. 68:2091-2102; Mazanet, M. M. et al. (2002) “B7-H1 Is Expressed By Human Endothelial Cells And Suppresses T-Cell Cytokine Synthesis,” J. Immunol. 169:3581-3588), myocardium (Brown, J. A. et al. (2003) “Blockade Of Programmed Death-1 Ligands On Dendritic Cells Enhances T-Cell Activation And Cytokine Production,” J. Immunol. 170:1257-1266), syncyciotrophoblasts (Petroff, M. G. et al. (2002) “B7 Family Molecules: Novel Immunomodulators At The Maternal-Fetal Interface,” Placenta 23:S95-S101). The molecules are also expressed by resident macrophages of some tissues, by macrophages that have been activated with interferon (IFN)-γ or tumor necrosis factor (TNF)-α (Latchman, Y. et al. (2001) “PD-L2 Is A Second Ligand For PD-1 And Inhibits T-Cell Activation,” Nat. Immunol 2:261-268), and in tumors (Dong, H. (2003) “B7-H1 Pathway And Its Role In The Evasion Of Tumor Immunity,” J. Mol. Med. 81:281-287).

The interaction between B7-H1 and PD-1 has been found to provide a crucial negative costimulatory signal to T and B-cells (Martin-Orozco, N. et al. (2007) “Inhibitory Costimulation And Anti-Tumor Immunity,” Semin. Cancer Biol. 17(4):288-298) and functions as a cell death inducer (Ishida, Y. et al. (1992) “Induced Expression Of PD-1, A Novel Member Of The Immunoglobulin Gene Superfamily, Upon Programmed Cell Death,” EMBO J. 11:3887-3895; Subudhi, S. K. et al. (2005) “The Balance Of Immune Responses: Costimulation Verse Coinhibition,” J. Molec. Med. 83:193-202). More specifically, interaction between low concentrations of the PD-1 receptor and the B7-H1 ligand has been found to result in the transmission of an inhibitory signal that strongly inhibits the proliferation of antigen-specific CD8⁺ T-cells; at higher concentrations the interactions with PD-1 do not inhibit T-cell proliferation but markedly reduce the production of multiple cytokines (Sharpe, A. H. et al. (2002) “The B7-CD28 Superfamily,” Nature Rev. Immunol. 2:116-126). T-cell proliferation and cytokine production by both resting and previously activated CD4 and CD8 T-cells, and even naive T-cells from umbilical-cord blood, have been found to be inhibited by soluble B7-H1-Fc fusion proteins (Freeman, G. J. et al. (2000) “Engagement Of The PD-1 Immunoinhibitory Receptor By A Novel B7 Family Member Leads To Negative Regulation Of Lymphocyte Activation,” J. Exp. Med. 192:1-9; Latchman, Y. et al. (2001) “PD-L2 Is A Second Ligand For PD-1 And Inhibits T-Cell Activation,” Nature Immunol. 2:261-268; Carter, L. et al. (2002) “PD-1:PD-L Inhibitory Pathway Affects Both CD4(+) and CD8(+) T-cells And Is Overcome By IL-2,” Eur. J. Immunol. 32(3):634-643; Sharpe, A. H. et al. (2002) “The B7-CD28 Superfamily,” Nature Rev. Immunol. 2:116-126).

The role of B7-H1 and PD-1 in inhibiting T-cell activation and proliferation has suggested that these biomolecules might serve as therapeutic targets for treatments of inflammation and cancer. Thus, the use of anti-PD-1 antibodies to treat infections and tumors and to up-modulate an adaptive immune response has been proposed (see, United States Patent Application Publication Nos. 2010/0040614; 2010/0028330; 2004/0241745; 2008/0311117; 2009/0217401; U.S. Pat. Nos. 7,521,051; 7,563,869; 7,595,048; PCT Publication Nos. WO 2004/056875; WO 2008/083174). Antibodies capable of specifically binding to PD-1 have been reported by Agata, T. et al. (1996) “Expression Of The PD-1 Antigen On The Surface Of Stimulated Mouse T And B Lymphocytes,” Int. Immunol. 8(5):765-772; and Berger, R. et al. (2008) “Phase I Safety And Pharmacokinetic Study Of CT-011, A Humanized Antibody Interacting With PD-1, In Patients With Advanced Hematologic Malignancies,” Clin. Cancer Res. 14(10):3044-3051 (see, also, U.S. Pat. Nos. 8,008,449 and 8,552,154; US Patent Publications No. 2007/0166281; 2012/0114648; 2012/0114649; 2013/0017199; 2013/0230514 and 2014/0044738; and PCT Patent Publication Nos. WO 2003/099196; WO 2004/004771; WO 2004/056875; WO 2004/072286; WO 2006/121168; WO 2007/005874; WO 2008/083174; WO 2009/014708; WO 2009/073533; WO 2012/135408, WO 2012/145549; and WO 2013/014668).

However, despite all such prior advances, a need remains for improved compositions capable of more vigorously directing the body's immune system to attack cancer cells or pathogen-infected cells, especially at lower therapeutic concentrations and/or with reduced side effects. Although the adaptive immune system can be a potent defense mechanism against cancer and disease, it is often hampered by immune suppressive/evasion mechanisms in the tumor microenvironment, such as the expression of PD-1 and CTLA-4. Furthermore, co-inhibitory molecules expressed by tumor cells, immune cells, and stromal cells in the tumor milieu can dominantly attenuate T-cell responses against cancer cells. In addition, the use of anti-CTLA-4 antibodies induces well-identified side effects referred to as “immune-related adverse events” (irAEs). IrAEs include colitis/diarrhea, dermatitis, hepatitis, endocrinopathies, and inflammatory myopathy. These unique side effects are reported to arise due to breaking immune tolerance upon CTLA-4 blockade (Di Giacomo, A. M., et al. (2010) “The Emerging Toxicity Profiles Of Anti-CTLA-4 Antibodies Across Clinical Indications,” Semin Oncol. 37:499-507). Accordingly, therapies which overcome these limitations would be of great benefit.

As described in detail below, the present invention addresses this need by providing PD-1×CTLA-4 bispecific molecules. Such bispecific molecules are capable of binding to PD-1 and CTLA-4 molecules that are present on the surfaces of exhausted and tolerant tumor-infiltrating lymphocytes and other cell types, and of thereby impairing the ability of such cell-surface molecules to respond to their respective ligands. As such, the PD-1×CTLA-4 bispecific molecules of the present invention act to block PD-1- and CTLA-4-mediated immune system inhibition, so as to promote the activation or continued activation of the immune system. These attributes permit such bispecific molecules to have utility in stimulating the immune system and particularly in the treatment of cancer and pathogen-associated diseases and conditions. The present invention is directed to these and other goals.

### SUMMARY OF THE INVENTION

The present invention is directed to bispecific molecules (e.g., diabodies, bispecific antibodies, trivalent binding molecules, etc.) that possess at least one epitope-binding site that is immunospecific for an epitope of PD-1 and at least one epitope-binding site that is immunospecific for an epitope of CTLA-4 (i.e., a “PD-1×CTLA-4 bispecific molecule”). The present invention concerns such PD-1×CTLA-4 bispecific molecules that possess two epitope-binding sites that are immunospecific for one (or two) epitope(s) of PD-1 and two epitope-binding sites that are immunospecific for one (or two) epitope(s) of CTLA-4. The present invention also is directed to such PD-1×CTLA-4 bispecific molecules that additionally comprise an immunoglobulin Fc Region. The PD-1×CTLA-4 bispecific molecules of the present invention are capable of simultaneously binding to PD-1 and to CTLA-4, particularly as such molecules are arrayed on the surfaces of human cells. The invention is directed to pharmaceutical compositions that contain such PD-1×CTLA-4 bispecific molecules, and to methods involving the use of such bispecific molecules in the treatment of cancer and other diseases and conditions. The present invention also pertains to methods of using such PD-1×CTLA-4 bispecific molecules to stimulate an immune response.

In detail, the invention provides a bispecific molecule possessing both one or more epitope-binding sites capable of immunospecific binding to (an) epitope(s) of PD-1 and one or more epitope-binding sites capable of immunospecific binding to (an) epitope(s) of CTLA-4, wherein the molecule comprises:
(A) a Heavy Chain Variable Domain and a Light Chain Variable Domain of an antibody that binds PD-1; and
(B) a Heavy Chain Variable Domain and a Light Chain Variable Domain of an antibody that binds CTLA-4;
   wherein the bispecific binding molecule is:
(i) a diabody, the diabody being a covalently bonded complex that comprises two, three, four or five polypeptide chains; or
(ii) a trivalent binding molecule, the trivalent binding molecule being a covalently bonded complex that comprises three, four, five, or more polypeptide chains.

The invention concerns the embodiment of such bispecific molecules, wherein the bispecific binding molecule exhibits an activity that is enhanced relative to such activity exhibited by two monospecific molecules one of which possesses the Heavy Chain Variable Domain and the Light Chain Variable Domain of the antibody that binds PD-1 and the other of which possesses the Heavy Chain Variable Domain and the Light Chain Variable Domain of the antibody that binds CTLA-4.

The invention concerns the embodiment of all such bispecific molecules, wherein the molecule elicits fewer immune-related adverse events (irAEs) when administered to a subject in need thereof relative to such iREs elicited by the administration of a monospecific antibody that binds CTLA-4 such as ipilimumab.

The invention additionally concerns the embodiment of such bispecific molecules in which the molecule comprises an Fc Region. The invention additionally concerns the embodiment of such bispecific molecules wherein the Fc Region is a variant Fc Region that comprises:
(A) one or more amino acid modifications that reduces the affinity of the variant Fc Region for an FcγR; and/or
(B) one or more amino acid modifications that enhances the serum half-life of the variant Fc Region.

The invention additionally concerns the embodiment of such bispecific molecules wherein the modifications that reduces the affinity of the variant Fc Region for an FcγR comprise the substitution of L234A; L235A; or L234A and L235A, wherein the numbering is that of the EU index as in Kabat.

The invention additionally concerns the embodiment of such bispecific molecules wherein the modifications that that enhances the serum half-life of the variant Fc Region comprise the substitution of M252Y; M252Y and S254T; M252Y and T256E; M252Y, S254T and T256E; or K288D and H435K, wherein the numbering is that of the EU index as in Kabat.

The invention additionally concerns the embodiment of all such bispecific molecules wherein the molecule is the diabody and comprises two epitope-binding sites capable of immunospecific binding to an epitope of PD-1 and two epitope-binding sites capable of immunospecific binding to an epitope of CTLA-4.

The invention additionally concerns the embodiment of all such bispecific molecules wherein the molecule is the trivalent binding molecule and comprises two epitope-binding sites capable of immunospecific binding to an epitope of PD-1 and one epitope-binding site capable of immunospecific binding to an epitope of CTLA-4.

The invention additionally concerns the embodiment of all such bispecific molecules wherein the molecule is capable of binding to PD-1 and CTLA-4 molecules present on the cell surface.

The invention additionally concerns the embodiment of all such bispecific molecules wherein the molecule is capable of simultaneously binding to PD-1 and CTLA-4.

The invention additionally concerns the embodiment of all such bispecific molecules wherein the molecule promotes the stimulation of immune cells, and particularly wherein the stimulation of immune cells results in:
(A) immune cell proliferation; and/or
(B) immune cell production and/or release of at least one cytokine; and/or
(C) immune cell production and/or release of at least one lytic molecule; and/or
(D) immune cell expression of at least one activation marker.

The invention additionally concerns the embodiment of all such bispecific molecules wherein the immune cell is a T-lymphocyte or an NK-cell.

The invention additionally concerns the embodiment of all such bispecific molecules wherein the epitope-binding sites capable of immunospecific binding to an epitope of PD-1 comprise:
(A) the VH Domain of PD-1 mAb 1 (SEQ ID NO:47) and the VL Domain of PD-1 mAb 1 (SEQ ID NO:48); or
(B) the VH Domain of PD-1 mAb 2 (SEQ ID NO:49) and the VL Domain of PD-1 mAb 2 (SEQ ID NO:50); or
(C) the VH Domain of PD-1 mAb 3 (SEQ ID NO:51) and the VL Domain of PD-1 mAb 3 (SEQ ID NO:52); or
(D) the VH Domain of PD-1 mAb 4 (SEQ ID NO:53) and the VL Domain of PD-1 mAb 4 (SEQ ID NO:54); or
(E) the VH Domain of PD-1 mAb 5 (SEQ ID NO:55) and the VL Domain of PD-1 mAb 5 (SEQ ID NO:56); or
(F) the VH Domain of PD-1 mAb 6 (SEQ ID NO:57) and the VL Domain of PD-1 mAb 6 (SEQ ID NO:58); or
(G) the VH Domain of PD-1 mAb 6-I VH (SEQ ID NO:86) and the VL Domain of PD-1 mAb 6-SQ VL (SEQ ID NO:87); or
(H) the VH Domain of PD-1 mAb 7 (SEQ ID NO:59) and the VL Domain of PD-1 mAb 7 (SEQ ID NO:60); or
(I) the VH Domain of PD-1 mAb 8 (SEQ ID NO:61) and the VL Domain of PD-1 mAb 8 (SEQ ID NO:62).

The invention additionally concerns the embodiment of all such bispecific molecules wherein the epitope-binding site(s) capable of immunospecific binding to an epitope of CTLA-4 comprise:
(A) the VH Domain of CTLA-4 mAb 1 (SEQ ID NO:76) and the VL Domain of CTLA-4 mAb 1 (SEQ ID NO:77); or
(B) the VH Domain of CTLA-4 mAb 2 (SEQ ID NO:78) and the VL Domain of CTLA-4 mAb 2 (SEQ ID NO:79); or
(C) the VH Domain of CTLA-4 mAb 3 (SEQ ID NO:90) and the VL Domain of CTLA-4 mAb 3 (SEQ ID NO:91).

The invention additionally concerns the embodiment of such bispecific molecules wherein:
(A) the epitope-binding sites capable of immunospecific binding to an epitope of PD-1 comprise the VH Domain of PD-1 mAb 6-I VH (SEQ ID NO:86) and the VL Domain of PD-1 mAb 6-SQ (SEQ ID NO:87); and
(B) the epitope-binding site(s) capable of immunospecific binding to an epitope of CTLA-4 comprise(s) the VH Domain of CTLA-4 mAb 3 (SEQ ID NO:90) and the VL Domain of CTLA-4 mAb 3 (SEQ ID NO:91).

The invention additionally concerns the embodiment of all such bispecific molecules wherein the molecule comprises:
(A) two polypeptide chains having SEQ ID NO:95, and two polypeptide chain having SEQ ID NO:96; or
(B) two polypeptide chains having SEQ ID NO:97, and two polypeptide chain having SEQ ID NO:98; or
(C) two polypeptide chains having SEQ ID NO:99, and two polypeptide chain having SEQ ID NO:100; or
(D) two polypeptide chains having SEQ ID NO:102, and two polypeptide chain having SEQ ID NO:103; or
(E) two polypeptide chains having SEQ ID NO:101, and two polypeptide chain having SEQ ID NO:100; or
(F) one polypeptide chains having SEQ ID NO:104, one polypeptide chain having SEQ ID NO:105, one polypeptide chain having SEQ ID NO:106, and one polypeptide chain having SEQ ID NO:107; or
(G) one polypeptide chains having SEQ ID NO:108, one polypeptide chain having SEQ ID NO:105, one polypeptide chain having SEQ ID NO:109, and one polypeptide chain having SEQ ID NO:107.

The invention additionally concerns the embodiment of such bispecific molecules in which the molecule comprises an Albumin-Binding Domain, and especially a deimmunized Albumin-Binding Domain.

The invention additionally concerns a pharmaceutical composition that comprises an effective amount of any of such bispecific molecules and a pharmaceutically acceptable carrier.

The invention additionally concerns the use of such pharmaceutical composition or the use of any of the above-described bispecific molecules to promote stimulation of an immune-mediated response of a subject in need thereof, and in particular, wherein such molecule promotes the stimulation of immune cells, and in particular, stimulation of NK-cells and/or T-lymphocytes. The invention particularly concerns the embodiments wherein such stimulation results in immune cell proliferation, immune cell production and/or release of cytokines (e.g., IFNγ, IL-2, TNFα, etc.), immune cell production and/or release of lytic molecules (e.g., granzyme, perforin, etc.), and/or immune cell expression of activation markers (e.g., CD69, CD25, CD107a, etc.). The invention further concerns methods of treating cancer or other diseases that involve the use or administration of any of the above-described PD-1×CTLA-4 bispecific molecules to stimulate an immune mediated response. The invention particularly concerns the embodiments in which the immune stimulatory activity of any of the above-described PD-1×CTLA-4 bispecific molecules is more potent than the joint or combined administration of a separate anti-PD-1 antibody and a separate anti-CTLA-4 antibody (especially, wherein such antibodies are monospecific for such molecules). The invention also concerns embodiments in which immune cells, particularly NK-cells and/or T-lymphocytes, stimulated by the above-described PD-1×CTLA-4 bispecific molecules exhibit enhanced proliferation, altered production and/or release of cytokines (e.g., IFNγ, IL-2, TNFα, etc.), altered production and/or release of lytic molecules, and/or altered expression of activation markers relative to that exhibited by such cells stimulated by the joint or combined administration of a separate anti-PD-1 antibody and a separate anti-CTLA-4 antibody. The invention also concerns embodiments in which the above-described PD-1×CTLA-4 bispecific molecules have a reduced incidence of irAEs. The invention additionally concerns the embodiments in which any of the above-described PD-1×CTLA-4 bispecific molecules are used in the treatment of a disease or condition associated with a suppressed immune system, especially cancer or an infection.

The invention additionally concerns such a use to treat a disease or condition associated with a suppressed immune system, or in the treatment of such a disease or condition. The invention particularly concerns such a use in in the treatment of a disease or condition associated with a suppressed immune system, or wherein the disease or condition is cancer or an infection (particularly, an infection characterized by the presence of a bacterial, fungal, viral or protozoan pathogen).

The invention particularly concerns such a use wherein:
(A) the use is in the treatment of cancer, and the cancer is characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumor, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, a hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer; or (B) the use is in the treatment of infection, and the infection is a chronic viral, bacterial, fungal and parasitic infection, characterized the presence of Epstein Barr virus, Hepatitis A Virus (HAV); Hepatitis B Virus (HBV); Hepatitis C Virus (HCV); herpes viruses (e.g. HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia lambia, trichinella, Dientamoeba Fragilis, Trypanosoma brucei, Trypanosoma cruzi, or Leishmania donovani.

The invention particularly concerns such use in the treatment of cancer, wherein the cancer is colorectal cancer, hepatocellular carcinoma, glioma, kidney cancer, breast cancer, multiple myeloma, bladder cancer, neuroblastoma; sarcoma, non-Hodgkin's lymphoma, non-small cell lung cancer, ovarian cancer, pancreatic cancer, a rectal cancer, acute myeloid leukemia (AML), chronic myelogenous leukemia (CML), acute B lymphoblastic leukemia (B-ALL), chronic lymphocytic leukemia (CLL), hairy cell leukemia (HCL), blastic plasmacytoid dendritic cell neoplasm (BPDCN), non-Hodgkin's lymphomas (NHL), including mantel cell leukemia (MCL), and small lymphocytic lymphoma (SLL), Hodgkin's lymphoma, systemic mastocytosis, or Burkitt's lymphoma.

### BRIEF DESCRIPTION OF THE DRAWINGS

FIG. 1 provides a schematic of a representative covalently bonded diabody having two epitope-binding sites composed of two polypeptide chains, each having an E-coil or K-coil Heterodimer-Promoting Domain (alternative Heterodimer-Promoting Domains are provided below). A cysteine residue may be present in a linker and/or in the Heterodimer-Promoting Domain as shown in FIG. 3B. VL and VH Domains that recognize the same epitope are shown using the same shading or fill pattern.

FIG. 2 provides a schematic of a representative covalently bonded diabody molecule having two epitope-binding sites composed of two polypeptide chains, each having a CH2 and CH3 Domain, such that the associated chains form all or part of an Fc Region. VL and VH Domains that recognize the same epitope are shown using the same shading or fill pattern.

FIGS. 3A-3C provide schematics showing representative covalently bonded tetravalent diabodies having four epitope-binding sites composed of two pairs of polypeptide chains (i.e., four polypeptide chains in all). One polypeptide of each pair possesses a CH2 and CH3 Domain, such that the associated chains form all or part of an Fc Region. VL and VH Domains that recognize the same epitope are shown using the same shading or fill pattern. The two pairs of polypeptide chains may be same. In such embodiments wherein the two pairs of polypeptide chains are the same and the VL and VH Domains recognize different epitopes (as shown in FIGS. 3A-3B), the resulting molecule possesses four epitope-binding sites and is bispecific and bivalent with respect to each bound epitope. In such embodiments wherein the VL and VH Domains recognize the same epitope (e.g., the same VL Domain CDRs and the same VH Domain CDRs are used on both chains) the resulting molecule possesses four epitope-binding sites and is monospecific and tetravalent with respect to a single epitope. Alternatively, the two pairs of polypeptides may be different. In such embodiments wherein the two pairs of polypeptide chains are different and the VL and VH Domains of each pair of polypeptides recognize different epitopes (as shown by the different shading and patterns in FIG. 3C), the resulting molecule possesses four epitope-binding sites and is tetraspecific and monovalent with respect to each bound epitope. FIG. 3A shows an Fc Region-containing diabody which contains a peptide Heterodimer-Promoting Domain comprising a cysteine residue. FIG. 3B shows an Fc Region-containing diabody, which contains E-coil and K-coil Heterodimer-Promoting Domains comprising a cysteine residue and a linker (with an optional cysteine residue). FIG. 3C, shows an Fc-Region-Containing diabody, which contains antibody CH1 and CL domains.

FIGS. 4A and 4B provide schematics of a representative covalently bonded diabody molecule having two epitope-binding sites composed of three polypeptide chains. Two of the polypeptide chains possess a CH2 and CH3 Domain, such that the associated chains form all or part of an Fc Region. The polypeptide chains comprising the VL and VH Domain further comprise a Heterodimer-Promoting Domain. VL and VH Domains that recognize the same epitope are shown using the same shading or fill pattern.

FIG. 5 provides the schematics of a representative covalently bonded diabody molecule having four epitope-binding sites composed of five polypeptide chains. Two of the polypeptide chains possess a CH2 and CH3 Domain, such that the associated chains form an Fc Region that comprises all or part of an Fc Region. The polypeptide chains comprising the linked VL and VH Domains further comprise a Heterodimer-Promoting Domain. VL and VH Domains that recognize the same epitope are shown using the same shading or fill pattern.

FIGS. 6A-6F provide schematics of representative Fc Region-containing trivalent binding molecules having three epitope-binding sites. FIGS. 6A and 6B, respectively, illustrate schematically the domains of trivalent binding molecules comprising two diabody-type binding domains and a Fab-type binding domain having different domain orientations in which the diabody-type binding domains are N-terminal or C-terminal to an Fe Region. The molecules in FIGS. 6A and 6B comprise four chains. FIGS. 6C and 6D, respectively, illustrate schematically the domains of trivalent binding molecules comprising two diabody-type binding domains N-terminal to an Fc Region, and a linked Fab-type binding domain, or an scFv-type binding domain. The trivalent binding molecules in FIGS. 6E and 6F, respectively illustrate schematically the domains of trivalent binding molecules comprising two diabody-type binding domains C-terminal to an Fc Region, and a Fab-type binding domain in which the light chain and heavy chain are linked via a polypeptide spacer, or an scFv-type binding domain. The trivalent binding molecules in FIGS. 6C-6F comprise three chains. VL and VH Domains that recognize the same epitope are shown using the same shading or fill pattern.

FIG. 7 illustrates the principles of the present invention by showing that an exemplary bispecific molecule (a PD-1×LAG-3 bispecific molecule, designated as DART A) is able to stimulate cytokine production to levels higher than those observed upon the joint or combined administration of the parental anti-PD-1 and anti-LAG-3 antibodies. Shown are IFNγ secretion profiles of PBMCs from a representative donor, stimulated with SEB (0.5 ng/mL) and treated with the exemplary bispecific molecule (PD-1×LAG-3 bispecific molecule DART A) or with the anti-PD-1 and anti-LAG-3 antibodies alone or in combination.

FIGS. 8A-8D show the results of ELISA studies measuring the binding of serially diluted binding molecules to human CTLA-4 and human PD-1. FIGS. 8A-8B show the binding curves of CTLA-4 mAb 3 G4P, DART D, TRIDENT A or DART B to soluble hCTLA-4-Avi-His (1 μg/mL) (FIG. 8A) or hPD-1-His (1 μg/mL) (FIG. 8B) that had been coated onto support plates. Goat anti-human-Fc-HRP (1:10,000) was employed as the secondary detection molecule to detect binding. FIGS. 8C-8D show the results of a study on the effect of altering orientations and binding domains on binding. PD-1×CTLA-4 bispecific molecules comprising the CTLA-4 binding domains of CTLA-4 mAb 1 (e.g., DART B) and CTLA-4 mAb 3 (e.g., DART C and DART D) were incubated in the presence of soluble human PD-1 (FIG. 8C) or soluble human CTLA-4-Avi-His (FIG. 8D), that had been coated onto support plates. Goat anti-human-Fcγ-HRP was employed as the secondary detection molecule to detect binding using PICO chemiluminescent substrate.

FIGS. 9A-9E show the results of an evaluation of the ability of DART D, TRIDENT A, PD-1 mAb 6 G4P, and CTLA-4 mAb 3 G4P and a control trident (having two binding sites for RSV and one binding site for CTLA-4) to block binding ligand binding to PD-1 and CTLA-1, alone and in combination. Blockade of PD-L1 binding to PD-1 was evaluated in the presence of equal amounts of an irrelevant antigen (FIG. 9A) and in the presence of equal amounts of CTLA-4 (FIG. 9B), and blockade of B7-1 binding to CTLA-4 was evaluated evaluated in the presence of equal amounts of an irrelevant antigen (FIG. 9C) and in the presence of equal amounts of PD-1 (FIG. 9D) and in the presence of four fold more PD-1 (FIG. 9E) using an ELISA assay.

FIGS. 10A-10B show the results of an evaluation of the ability of DART B, DART D, TRIDENT A, the anti-CTLA-4 antibodies CTLA-4 mAb 1, CTLA-4 mAb 3 G4P, and an hIgG control antibody to bind to CHO cells expressing cynomolgus monkey CTLA-4 (FIG. 10A) or human CTLA-4 (FIG. 10B). Binding was detected using an anti-human Fc secondary antibody.

FIGS. 11A-11B show the results of an evaluation of the ability of DART C, DART D, DART E, TRIDENT A, the anti-CTLA-4 antibodies CTLA-4 mAb 1, CTLA-4 mAb 3 G1AA, and the anti-PD-1 antibody PD-1 mAb 6 G4P to bind to Jurkat cells (which express huCTLA-4 but not PD-1 on their surface). Binding of the DART and TRIDENT molecules to human CTLA-4 was detected using anti-human FC secondary Ab (FACS). FIG. 11A shows the results for DART C, DART D, DART E, CTLA-4 mAb 1, CTLA-4 mAb 3 G1AA, and PD-1 mAb 6 G4P. FIG. 11B shows the results for CTLA-4 mAb 1, CTLA-4 mAb 3 G1AA, PD-1 mAb 6 G4P and TRIDENT A.

FIGS. 12A-12B show the results of an evaluation of the ability of DART D, TRIDENT A and the anti-CTLA-4 antibodies CTLA-4 mAb 1, CTLA-4 mAb 3 G1AA to block the CTLA-4 ligands B7-1 and B7-2 in a cell-based assay. His-tagged derivatives of B7-1 and B7-2 were incubated in the presence of the Jurkat cells and artificial antigen presenting cells (Promega). Binding of His-B7-1 and His-B7-2 was detected using an anti-His antibody. The results of this evaluation are shown in FIG. 12A (His-B7-1) and FIG. 12B (His-B7-2).

FIG. 13 shows the results of an evaluation of the ability of DART C, DART D, TRIDENT A, CTLA-4 mAb 3 G1AA and PD-1 mAb 6 G4P to reverse CTLA-4 immune checkpoint inhibitory signal as demonstrated in a IL-2/Luc-Jurkat-CTLA-4 reporter assay by increased luciferase expression. IL-2/Luc-Jurkat-CTLA-4 cells were incubated in the presence of the listed binding molecules (R:S=1:0.3) for 30 min at 37° C., after which time artificial antigen presenting Raji cells were added and the incubation continued for 6 hours. Reversal of CTLA-4 immune checkpoint inhibitory signal was determined by the luciferase assay.

FIG. 14 shows the results of an evaluation of the ability of DART D, TRIDENT A, PD-1 mAb 6 G4P, and CTLA-4 mAb 3 G1AA to bind NSO cells that express PD-1 but not CTLA-4. Binding molecules were incubated in the presence of the cells and the mean fluorescence index of the cells was measured.

FIGS. 15A-15B show the results of an evaluation of the ability of DART D, TRIDENT A, PD-1 mAb 6 G4P, and CTLA-4 mAb 3 G1AA to block binding between PD-1 and its ligands PD-L1 and PD-L2 in a cell based assay. PD-L1-PE or PD-L2-PE was incubated in the presence of such binding molecules and their ability to bind to NSO-PD-1 cells was evaluated using FACS. FIG. 15A (PD-L1); FIG. 15B (PD-L2).

FIG. 16 shows the results of an evaluation of the ability of DART D, TRIDENT A, CTLA-4 mAb 3 G1AA, and PD-1 mAb 6 G4P to block immune inhibition resulting from a PD-1/PD-L1 interaction. Binding molecules were incubated in the presence of PD-L1 CHO and Jurkat effector cells, and the ability of the binding molecules to block immune inhibition (by blocking the PD-1/PD-L1 interaction) was assessed by following the extent of CD3-mediated activation (as demonstrated by increased luciferase expression in the NFAT-luc/PD-1 Jurkat assay; Promega).

FIG. 17 shows the results of an evaluation of the ability of DART D, TRIDENT A, and a negative control antibody to co-ligate PD-1 and CTLA-4 in an enzyme-fragment complementation assay by DiscoverX. Aliquots of the U2OS CTLA-4(1-195)-PK PD-1(1-199)-EA cell line #9 were plated in quadruplicate at 5,000 cells/well in DiscoverX CP5 plating media on 384-well plates. Cells were allowed to attach for 4 hours at 37° C./5% CO₂. 11 point, 1:3 dilution series of each of the binding molecules were then added to the PD-1-CTLA-4 cells and the DART D and TRIDENT A samples were added to the PD-1-LAG-3 cells. The plates were incubated overnight (16 hrs) at 37° C./5% CO₂. PathHunter detection reagent was added to the wells, which were then incubated for 1 hour at room temperature in the dark, and the plate was then read on an Envision luminometer.

FIG. 18 shows the results of an evaluation of the ability of DART D, TRIDENT A, CTLA-4 mAb 3 G1AA, PD-1 mAb 6 G4P and the combinations of CTLA-4 mAb 3 G1AA/PD-1 mAb 6 G4P (Ab Combo 1) to enhance the response of a Mixed Lymphocyte Reaction. Monocyte-derived dendritic cells were generated by treating CD14+ monocytes with GM-CSF (provided at day 1 of the incubation period) and IL-4 (provided at day 7 of the incubation period). At day 8 of the incubation period, a MLR was set up by incubating the CD4+ T cells with the monocyte-derived dendritic cells (provided at day 8 of the incubation period) and the anti-CTLA-4 and anti-PD-1 binding molecules (provided at day 8 of the incubation period). The release of IFN-7 is plotted in FIG. 18. Both the bispecific DART D and TRIDENT A molecules were found to enhance the MLR response to the same extent or slightly better than the combination of individual parental antibodies. The presented data comprises seven series (each relating to a different binding molecule: hIgG4 control; PD-1 mAb 6 G4P; CTLA-4 mAb 3 G1AA; a combination of CTLA-4 mAb 3 G1AA/PD-1 mAb 6 G4P (Ab Combo 1); DART D; TRIDENT A; and an hIgG1 control, respectively from left to right); each series is composed of six columns (each relating to a different concentration of the provided molecule: 0.016, 0.08, 0.4, 2, 10 or 50 nM, respectively from left to right).

FIGS. 19A-19D show the effect of administration of DART D, TRIDENT A, CTLA-4 mAb 3 G1AA, PD-1 mAb 6 G4P and the combination of CTLA-4 mAb 1/PD-1 mAb 1 (Ab Combo 1) on T-cell responses using a Staphylococcus aureus enterotoxin type B (SEB) re-stimulation assay. FIGS. 19A-19B show fluorescence-activated cell sorting (FACS) dot plots of the expression of PD-1 vs. CTLA-1 by such PBMCs in the absence (FIG. 19A) or presence (FIG. 19B) of SEB stimulation. FIG. 19C shows the effect of the SEB stimulation on IFN-7 secretion. PBMCs were stimulated with Staphylococcus aureus enterotoxin type B (SEB) at 0.5 ng/ml for 48 hours. Cells were then harvested, washed and re-plated in 96 well plates with antibodies at various concentrations with fresh SEB for an additional 48 hours. The supernatant was then harvested and analyzed by flow cytometry ELISA for IFN-7 production. Both the bispecific DART and the TRIDENT protein showed an increase in IFN-7 response that recapitulated the response observed with the combination of the individual parental mAbs. Similar results were seen in a SEB Stimulation assay in which the PBMCs were cultured with a high concentration (500 ng/mL) of SEB for 72 hours. Presented are six series, each relating to a different binding molecule. Each series is composed of seven columns, which relate to the result obtained with 25 nM, 6.25 nM, 1.56 nM, 0.39 nM, 0.09 nM, 0.02 nM or 0.006 nM binding molecule (respectively, from left to right). FIG. 19D shows the release of IL-2 for a representative donor. PBMCs were stimulated with 0.5 ng/ml SEB for 48 hours, harvested, washed and re-plated in 96-well plates with fresh SEB and either DART D, TRIDENT A, CTLA-4 mAb 3 G1AA, PD-1 mAb 6 G4P or the combination of CTLA-4 mAb 3 G1AA/PD-1 mAb 6 G4P (Ab Combo 1) for an additional 48 hours, and the released TL-2 was measured. Presented are seven series, each relating to a different binding molecule or condition. Each series is composed of three columns, which relate to the result obtained with 0.5 nM, 5 nM or 50 nM binding molecule (respectively, from left to right). When antibodies were used in combination, each antibody was added at the indicated concentration so that the total concentration of antibody added is doubled.

FIGS. 20A-20B show the activity of a PD-1×CTLA-4 bispecific molecule in a PBMC implanted NOG murine model of Graft Versus Host Disease (GVHD). CD3+ T cell counts were performed via FACS on study day (FIG. 20A) on mice that had received DART D at a dose of 50 mg/kg or 500 mg/kg (FIG. 20A). Survival was monitored over the course of the study and is plotted as percent survival in FIG. 20B.

FIGS. 21A-21C show serum concentration-time profiles for cynomolgus monkeys (coded using a 6-character alphanumeric code) that had received DART D at 50 mg/kg on days 1, 8 and 15 of the study (FIG. 21A), DART D at 75 mg/kg on days 1, 8 and 15 of the study (FIG. 21B) or Trident A at 5 mg/kg on day 1 (FIG. 21C).

FIGS. 22A-22B show the effect of administration of DART D on absolute lymphocyte count (ALC) in treated cynomolgus monkeys. FIG. 22A shows the ALC in thousands of cells/μl (th/μl). FIG. 22B shows the percent change in the ALC normalized to Day 1 (D1).

FIGS. 23A-23B show CD4+ T cell proliferation and PD-1 occupancy on T cells in cynomolgus monkeys that had received DART D administered at 50 mg/kg (FIG. 23A) or DART D administered at 75 mg/kg (FIG. 23B).

FIGS. 24A-24B show the effect of DART D administration on CD4+ T cell proliferation in cynomolgus monkeys that had received DART D administered at 50 mg/kg (FIG. 24A) or DART D administered at 75 mg/kg (FIG. 24B).

### DETAILED DESCRIPTION OF THE INVENTION

The present invention is directed to bispecific molecules (e.g., diabodies, bispecific antibodies, trivalent binding molecules, etc.) that possess at least one epitope-binding site that is immunospecific for an epitope of PD-1 and at least one epitope-binding site that is immunospecific for an epitope of CTLA-4 (i.e., a “PD-1×CTLA-4 bispecific molecule”). The present invention concerns such PD-1×CTLA-4 bispecific molecules that possess two epitope-binding sites that are immunospecific for one (or two) epitope(s) of PD-1 and two epitope-binding sites that are immunospecific for one (or two) epitope(s) of CTLA-4. The present invention also is directed to such PD-1×CTLA-4 bispecific molecules that additionally comprise an immunoglobulin Fc Region. The PD-1×CTLA-4 bispecific molecules of the present invention are capable of simultaneously binding to PD-1 and to CTLA-4, particularly as such molecules are arrayed on the surfaces of human cells. The invention is directed to pharmaceutical compositions that contain such PD-1×CTLA-4 bispecific molecules, and to methods involving the use of such bispecific molecules in the treatment of cancer and other diseases and conditions. The present invention also pertains to methods of using such PD-1×CTLA-4 bispecific molecules to stimulate an immune response.

T-cell activation requires two distinct signals (Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). The first signal is provided by a T-Cell Receptor (TCR) molecule, expressed on the surface of a T-cell, that has recognized a peptide antigen that has become associated with a human leukocyte antigen (HLA) expressed on the surface of an Antigen-Presenting Cell (APC). The second signal is provided by the interaction of cognate pairs of co-stimulatory ligands: B7-1 and B7-2 expressed on APCs and their corresponding receptors: CD28 and CTLA-4 expressed on T-cells.

The binding of B7-1 and B7-2 molecules to CD28 stimulates T-cell proliferation and additionally induces increased expression of CTLA-4. CTLA-4 is a negative-regulator that competes with B7-1 and B7-2 for binding to CD28. Thus, the process responds to disease in two phases: the initial phase involves stimulating T-cell proliferation; the subsequent phase “winds down” the immune response and returns the subject to a quiescent immune state. Antibodies that bind CD28 can mimic the binding of B7-1 or B7-2 and thus induce or enhance T-cell effector function and the generation of tumor eradicating immunity; such antibodies are co-stimulatory. Conversely, antibodies that block CTLA-4 from binding to B7-1 and B7-2 can prevent T-cells from returning to a quiescent state; such T-cells thus maintain a sustained proliferation that can lead to autoimmunity and the development of immune-related adverse events” (irAEs) (Wang, L. et al. (Mar. 7, 2011) “VISTA, A Novel Mouse Ig Superfamily Ligand That Negatively Regulates T-Cell Responses,” J. Exp. Med. 10.1084/jem.20100619:1-16; Lepenies, B. et al. (2008) “The Role Of Negative Costimulators During Parasitic Infections,” Endocrine, Metabolic & Immune Disorders—Drug Targets 8:279-288). Of particular importance is binding between the B7.1 (CD80) and B7.2 (CD86) ligands of the Antigen-Presenting Cell and the CD28 and CTLA-4 receptors of the CD4⁺ T lymphocyte (Sharpe, A. H. et al. (2002) “The B7-CD28 Superfamily,” Nature Rev. Immunol. 2:116-126; Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321). Binding of B7.1 or of B7.2 to CD28 stimulates T-cell activation; binding of B7.1 or B7.2 to CTLA-4 inhibits such activation (Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321; Greenwald, R. J. et al. (2005) “The B7 Family Revisited,” Ann. Rev. Immunol. 23:515-548). CD28 is constitutively expressed on the surface of T-cells (Gross, J., et al. (1992) “Identification And Distribution Of The Costimulatory Receptor CD28 In The Mouse,” J. Immunol. 149:380-388), whereas CTLA-4 expression is rapidly upregulated following T-cell activation (Linsley, P. et al. (1996) “Intracellular Trafficking Of CTLA4 And Focal Localization Towards Sites Of TCR Engagement,” Immunity 4:535-543). Since CTLA-4 is the higher affinity receptor (Sharpe, A. H. et al. (2002) “The B7-CD28 Superfamily,” Nature Rev. Immunol. 2:116-126) binding first initiates T-cell proliferation (via CD28) and then inhibits it (via nascent expression of CTLA-4), thereby dampening the effect when proliferation is no longer needed.

In parallel with the above-described interactions, a second set of receptors and binding ligands function to inhibit the immune system, thereby serving as a brake to slow the CD28/B7-1/B7-2-mediated enhancement of the immune response. This auxiliary response involves the binding of the programmed cell death-1 protein (PD-1) receptor, expressed on the surface of T-cells, to corresponding ligands: PD-L1, expressed on Antigen-Presenting Cells (APCs) and PD-L2, expressed on epithelial cells (Chen L. et al. (2013) “Molecular Mechanisms Of T-Cell Co-Stimulation And Co-Inhibition,” Nature Reviews Immunology 13(4):227-242). In contrast to agonist antibodies that bind to CD28 to directly stimulate T-cell responses, antibodies that bind to either PD-1 or PD-L1 antagonize or block PD-1/PD-L1 engagement and thus maintain T-cell activation by preventing the delivery of a negative signal to the T-cell. As such, antibodies that bind to either PD-1 or PD-L1 augment or maintain T-cell proliferation, cytotoxicity, and/or cytokine secretion. Taken together agonist antibodies, such as anti-CD28, target positive signal pathways and are therefore co-stimulators, while antagonistic antibodies, such as anti-CTLA-4 and anti-PD-1, target negative signal pathways and are called checkpoint inhibitors.

As provided above, CTLA-4 and PD-1 represent the canonical checkpoint inhibitors which exert distinct inhibitory effects on T-cell activation. The PD-1×CTLA-4 bispecific molecules of the present invention are capable of binding to PD-1 and CTLA-4 cell-surface molecules that are present on the surfaces of lymphocytes, and of thereby impairing the ability of such cell-surface molecules to respond to their respective receptors. Without being bound by by any theory or mechanism, the inventors believe that PD-1 binding can release T-cell inhibition (e.g., at tumor sites and/or as a result of infection) and that CTLA-1 binding can stimulate polyclonal activation and stimulation. As such, the PD-1×CTLA-4 bispecific molecules of the present invention are able to attenuate PD-1 and CTLA-4-mediated immune system inhibition, and promote continued immune system activation. It has been demonstrated herein that bispecific molecules which target two immunomodulatory pathways are more potent than the combination of separate antibodies. The instant invention also provides PD-1×CTLA-4 bispecific molecules having PD-1:CTLA-4 binding ratios of 1:1, 1:2, 2:2 and 2:1 which allow for full blockade of both PD-1 and CTLA-4 as well as blockade that is biased toward CTLA-4 when co-expressed with PD-1. Accordingly, the PD-1×CTLA-4 bispecific molecules of the present invention provide unexpected superiority as compared to the combination of separate anti-PD-1 and anti-CTLA-4 antibodies. Additionally, the PD-1×CTLA-4 bispecific molecules of the present invention may provide immune stimulation with reduced risk of irAEs.

### I. Antibodies and Their Binding Domains

The antibodies of the present invention are immunoglobulin molecules capable of specific binding to a target, such as a carbohydrate, polynucleotide, lipid, polypeptide, etc., through at least one antigen recognition site, located in the Variable Domain of the immunoglobulin molecule. As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. As used herein, an Fc Region is said to be of a particular IgG isotype, class or subclass if its amino acid sequence is most homologous to that isotype relative to other IgG isotypes. In addition to their known uses in diagnostics, antibodies have been shown to be useful as therapeutic agents. Antibodies are capable of immunospecifically binding to a polypeptide or protein or a non-protein molecule due to the presence on such molecule of a particular domain or moiety or conformation (an “epitope”). An epitope-containing molecule may have immunogenic activity, such that it elicits an antibody production response in an animal; such molecules are termed “antigens”. The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Over 200 antibody-based drugs have been approved for use or are under development.

The term “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂ Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells may allow antigens to be better detected than ruptured cells by the immunized animal. Use of denaturing or harsh adjuvants, e.g., Freud's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody, while swapping the non-human remainder of the antibody with human antibody sequences.

Natural antibodies (such as IgG antibodies) are composed of two Light Chains complexed with two Heavy Chains. Each Light Chain contains a Variable Domain (VL) and a Constant Domain (CL). Each Heavy Chain contains a Variable Domain (VH), three Constant Domains (CH1, CH2 and CH3), and a Hinge Region located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Region. Thus, the structure of the light chains of an IgG molecule is n-VL-CL-c and the structure of the IgG heavy chains is n-VH-CH1-H-CH2-CH3-c (where H is the Hinge Region, and n and c represent, respectively, the N-terminus and the C-terminus of the polypeptide). The Variable Domains of an IgG molecule consist of the complementarity determining regions (CDR), which contain the residues in contact with epitope, and non-CDR segments, referred to as framework segments (FR), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of an antibody Light Chain are herein respectively designated CDR_{L}1 Domain, CDR_{L}2 Domain, and CDR_{L}3 Domain. Similarly, polypeptides that are (or may serve as) the first, second and third CDR of an antibody heavy chain are herein respectively designated CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or a diabody or a single-chain binding molecule (e.g., an scFv, a BiTe, etc.), or is another type of protein. Accordingly, as used herein, the term “epitope-binding fragment” means a fragment of an antibody capable of immunospecifically binding to an epitope, and the term “epitope-binding site” refers to a portion of a molecule comprising an epitope-binding fragment. An epitope-binding fragment may contain 1, 2, 3, 4, 5 or all 6 of the CDR Domains of such antibody and, although capable of immunospecifically binding to such epitope, may exhibit an immunospecificity, affinity or selectivity toward such epitope that differs from that of such antibody. Preferably, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an Fab₂ fragment, etc.). Unless specifically noted, the order of domains of the protein molecules described herein is in the N-terminal to C-Terminal direction.

The invention particularly encompasses PD-1×CTLA-4 bispecific binding molecules comprising one, two, or more than two single-chain Variable Domain fragments (“scFv”) of an anti-PD-1 antibody and one, two, or more than two single-chain Variable Domain fragments of an anti-CTLA-4 antibody. Single-chain Variable Domain fragments are made by linking Light and Heavy chain Variable Domains using a short linking peptide. Linkers can be modified to provide additional functions, such as to permit the attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention also particularly encompasses PD-1×CTLA-4 bispecific molecules comprising humanized anti-PD-1 and anti-CTLA-4 antibodies. The term “humanized” antibody refers to a chimeric molecule, generally prepared using recombinant techniques, having an antigen-binding site of an immunoglobulin from a non-human species and a remaining immunoglobulin structure of the molecule that is based upon the structure and/or sequence of a human immunoglobulin. The polynucleotide sequence of the variable domains of such antibodies may be used for genetic manipulation to generate such derivatives and to improve the affinity, or other characteristics of such antibodies. The general principle in humanizing an antibody involves retaining the basic sequence of the antigen-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains; (2) designing the humanized antibody or caninized antibody, i.e., deciding which antibody framework region to use during the humanizing or canonizing process; (3) the actual humanizing or caninizing methodologies/techniques; and (4) the transfection and expression of the humanized antibody. See, for example, U.S. Pat. Nos. 4,816,567; 5,807,715; 5,866,692; and 6,331,415.

The antigen-binding site may comprise either a complete Variable Domain fused onto Constant Domains or only the complementarity determining regions (CDRs) of such Variable Domain grafted to appropriate framework regions. Antigen-binding sites may be wild-type or modified by one or more amino acid substitutions. This eliminates the constant region as an immunogen in human individuals, but the possibility of an immune response to the foreign variable domain remains (LoBuglio, A. F. et al. (1989) “Mouse Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the variable domains as well so as to reshape them as closely as possible to human form. It is known that the variable domains of both heavy and light chains contain three complementarity determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable domains can be “reshaped” or “humanized” by grafting CDRs derived from non-human antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

A number of “humanized” antibody molecules comprising an antigen-binding site derived from a non-human immunoglobulin have been described, including chimeric antibodies having rodent or modified rodent Variable Domain and their associated complementarity determining regions (CDRs) fused to human constant domains (see, for example, Winter et al. (1991) “Man-made Antibodies,” Nature 349:293-299; Lobuglio et al. (1989) “Mouse/Human Chimeric Monoclonal Antibody In Man: Kinetics And Immune Response,” Proc. Natl. Acad. Sci. (U.S.A.) 86:4220-4224 (1989), Shaw et al. (1987) “Characterization Of A Mouse Human Chimeric Monoclonal Antibody (17-4A) To A Colon Cancer Tumor-Associated Antigen,” J. Immunol. 138:4534-4538, and Brown et al. (1987) “Tumor-Specific Genetically Engineered Murine Human Chimeric Monoclonal Antibody,” Cancer Res. 47:3577-3583). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692.

### II. Fcγ Receptors (FcγRs)

The CH2 and CH3 Domains of the two heavy chains interact to form the Fc Region, which is a domain that is recognized by cellular Fc Receptors, including but not limited to Fc gamma Receptors (FcγRs). As used herein, the term “Fc Region” is used to define a C-terminal region of an IgG heavy chain. The amino acid sequence of the CH2-CH3 Domain of an exemplary human IgG1 is (SEQ ID NO:1):

```
231 240 250 260 270
APELLGGPSV FLFPPKPKDT LMISRTPEVT CVVVDVSHED
              280 290 300 310
PEVKFNWYVD GVEVHNAKTK PREEQYNSTY RVVSVLTVLH
              320 330 340 350
QDWLNGKEYK CKVSNKALPA PIEKTISKAK GQPREPQVYT
              360 370 380 390
LPPSREEMTK NQVSLTCLVK GFYPSDIAVE WESNGQPENN
              400 410 420 430
YKTTPPVLDS DGSFFLYSKL TVDKSRWQQG NVFSCSVMHE
              440 447
ALHNHYTQKS LSLSPGX
```

as numbered by the EU index as set forth in Kabat, wherein X is a lysine (K) or is absent.

The amino acid sequence of the CH2-CH3 Domain of an exemplary human IgG2 is (SEQ ID NO:2):

```
231 240 250 260 270
APPVA-GPSV FLFPPKPKDT LMISRTPEVT CVVVDVSHED
              280 290 300 310
PEVQFNWYVD GVEVHNAKTK PREEQFNSTF RVVSVLTVVH
              320 330 340 350
QDWLNGKEYK CKVSNKGLPA PIEKTISKTK GQPREPQVYT
              360 370 380 390
LPPSREEMTK NQVSLTCLVK GFYPSDISVE WESNGQPENN
              400 410 420 430
YKTTPPMLDS DGSFFLYSKL TVDKSRWQQG NVFSCSVMHE
              440 447
ALHNHYTQKS LSLSPGX
```

as numbered by the EU index as set forth in Kabat, wherein X is a lysine (K) or is absent.

The amino acid sequence of the CH2-CH3 Domain of an exemplary human IgG3 is (SEQ ID NO:3):

```
231 240 250 260 270
APELLGGPSV FLFPPKPKDT LMISRTPEVT CVVVDVSHED
              280 290 300 310
PEVQFKWYVD GVEVHNAKTK PREEQYNSTF RVVSVLTVLH
              320 330 340 350
QDWLNGKEYK CKVSNKALPA PIEKTISKTK GQPREPQVYT
              360 370 380 390
LPPSREEMTK NQVSLTCLVK GFYPSDIAVE WESSGQPENN
              400 410 420 430
YNTTPPMLDS DGSFFLYSKL TVDKSRWQQG NIFSCSVMHE
              440 447
ALHNRFTQKS LSLSPGX
```

as numbered by the EU index as set forth in Kabat, wherein X is a lysine (K) or is absent.

The amino acid sequence of the CH2-CH3 Domain of an exemplary human IgG4 is (SEQ ID NO:4):

```
231 240 250 260 270
APEFLGGPSV FLFPPKPKDT LMISRTPEVT CVVVDVSQED
              280 290 300 310
PEVQFNWYVD GVEVHNAKTK PREEQFNSTY RVVSVLTVLH
              320 330 340 350
QDWLNGKEYK CKVSNKGLPS SIEKTISKAK GQPREPQVYT
              360 370 380 390
LPPSQEEMTK NQVSLTCLVK GFYPSDIAVE WESNGQPENN
              400 410 420 430
YKTTPPVLDS DGSFFLYSRL TVDKSRWQEG NVFSCSVMHE
              440 447
ALHNHYTQKS LSLSLGX
```

as numbered by the EU index as set forth in Kabat, wherein X is a lysine (K) or is absent.

Throughout the present specification, the numbering of the residues in the constant region of an IgG heavy chain is that of the EU index as in Kabat et al., SEQUENCES OF PROTEINS OF IMMUNOLOGICAL INTEREST, 5^{th} Ed. Public Health Service, NH1, MD (1991) (“Kabat”), expressly incorporated herein by references. The term “EU index as in Kabat” refers to the numbering of the human IgG1 EU antibody. Amino acids from the Variable Domains of the mature heavy and light chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical structures for the hypervariable regions of immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the PD-1×CTLA-4 bispecific molecules of the invention. Specifically encompassed by the instant invention are PD-1×CTLA-4 bispecific molecules lacking the C-terminal residue of the CH3 Domain. Also specifically encompassed by the instant invention are such constructs comprising the C-terminal lysine residue of the CH3 Domain.

As stated above, the Fe Region of natural IgG antibodies is capable of binding to cellular Fc gamma Receptors (FcγRs). Such binding results in the transduction of activating or inhibitory signals to the immune system. The ability of such binding to result in diametrically opposing functions reflects structural differences among the different FcγRs, and in particular reflects whether the bound FcγR possesses an immunoreceptor tyrosine-based activation motif (ITAM) or an immunoreceptor tyrosine-based inhibitory motif (ITIM). The recruitment of different cytoplasmic enzymes to these structures dictates the outcome of the FcγR-mediated cellular responses. ITAM-containing FcγRs include FcγRI, FcγRIIA, FcγRIIIA, and activate the immune system when bound to an Fc Region. FcγRIIB is the only currently known natural ITIM-containing FcγR; it acts to dampen or inhibit the immune system when bound to an Fc Region. Human neutrophils express the FcγRIIA gene. FcγRIIA clustering via immune complexes or specific antibody cross-linking serves to aggregate ITAMs with receptor-associated kinases which facilitate ITAM phosphorylation. ITAM phosphorylation serves as a docking site for Syk kinase, the activation of which results in the activation of downstream substrates (e.g., PI₃K). Cellular activation leads to release of pro-inflammatory mediators. The FcγRIIB gene is expressed on B lymphocytes; its extracellular domain is 96% identical to FcγRIIA and binds IgG complexes in an indistinguishable manner. The presence of an ITIM in the cytoplasmic domain of FcγRIIB defines this inhibitory subclass of FcγR. Recently the molecular basis of this inhibition was established. When co-ligated along with an activating FcγR, the ITIM in FcγRIIB becomes phosphorylated and attracts the SH2 domain of the inositol polyphosphate 5′-phosphatase (SHIP), which hydrolyzes phosphoinositol messengers released as a consequence of ITAM-containing FcγR-mediated tyrosine kinase activation, consequently preventing the influx of intracellular Ca⁺⁺. Thus cross-linking of FcγRIIB dampens the activating response to FcγR ligation and inhibits cellular responsiveness. B-cell activation, B-cell proliferation and antibody secretion is thus aborted.

### III. Bispecific Antibodies, Multispecific Diabodies and DART® Diabodies

The ability of an antibody to bind an epitope of an antigen depends upon the presence and amino acid sequence of the antibody's VL and VH Domains. Interaction of an antibody's Light Chain and Heavy Chain and, in particular, interaction of its VL and VH Domains forms one of the two epitope-binding sites of a natural antibody, such as an IgG. Natural antibodies are capable of binding to only one epitope species (i.e., they are monospecific), although they can bind multiple copies of that species (i.e., exhibiting bivalency or multivalency).

The binding domains of an antibody, and of the PD-1×CTLA-4 bispecific molecules of the present invention, bind to epitopes in an “immunospecific” manner. As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind to a second target. As such, “immunospecific binding” does not necessarily require (although it can include) exclusive binding. Generally, but not necessarily, reference to binding means “immunospecific” binding. Two molecules are said to be capable of binding to one another in a “physiospecific” manner, if such binding exhibits the specificity with which receptors bind to their respective ligands.

One aspect of the present invention reflects the recognition that the functionality of antibodies can be enhanced by generating multispecific antibody-based molecules that can simultaneously bind to one or more epitope(s) of PD-1 and also one or more epitope(s) of CTLA-4. For molecules having more than one epitope-binding site immunospecific for an epitope of PD-1, such epitopes may be identical to one another, overlapping, or distinct from one another; binding to one such epitope may compete with or not compete with binding to another of such epitopes. Likewise, for molecules having more than one epitope-binding site immunospecific for an epitope of CTLA-4, such epitopes may be identical to one another, overlapping, or distinct from one another; binding to one such epitope may compete with or not compete with binding to the second of such epitopes. It is expressly contemplated that such characteristics may be independently varied to yield PD-1×CTLA-4 bispecific molecules that, for example, possess:
(1) the ability to bind to two identical epitopes of PD-1 and to:
   (a) two identical epitopes of CTLA-4; or
   (b) two overlapping epitopes of CTLA-4; or
   (c) two distinct epitopes of CTLA-4;
or
(2) the ability to bind to two overlapping epitopes of PD-1 and to:
   (a) two identical epitopes of CTLA-4; or
   (b) two overlapping epitopes of CTLA-4; or
   (c) two distinct epitopes of CTLA-4;
or
(3) the ability to bind to two distinct epitopes of PD-1 and to:
   (a) two identical epitopes of CTLA-4; or
   (b) two overlapping epitopes of CTLA-4; or
   (c) two distinct epitopes of CTLA-4.

In order to provide molecules having greater capability than natural antibodies, a wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further epitope-binding fragment (e.g., an scFv, VL, VH, etc.) to, or within the antibody core (IgA, IgD, IgE, IgG or IgM), or to fuse multiple epitope-binding fragments (e.g., two Fab fragments or scFvs). Alternative formats use linker peptides to fuse an epitope-binding fragment (e.g., an scFv, VL, VH, etc.) to a dimerization domain such as the CH2-CH3 Domain or alternative polypeptides (WO 2005/070966, WO 2006/107786A WO 2006/107617A, WO 2007/046893). PCT Publications Nos. WO 2013/174873, WO 2011/133886 and WO 2010/136172 disclose a trispecific antibody in which the CL and CH1 Domains are switched from their respective natural positions and the VL and VH Domains have been diversified (WO 2008/027236; WO 2010/108127) to allow them to bind to more than one antigen. PCT Publications Nos. WO 2013/163427 and WO 2013/119903 disclose modifying the CH2 Domain to contain a fusion protein adduct comprising a binding domain. PCT Publications Nos. WO 2010/028797, WO2010028796 and WO 2010/028795 disclose recombinant antibodies whose Fc Regions have been replaced with additional VL and VH Domains, so as to form trivalent binding molecules. PCT Publications Nos. WO 2003/025018 and WO2003012069 disclose recombinant diabodies whose individual chains contain scFv Domains. PCT Publications No. WO 2013/006544 discloses multivalent Fab molecules that are synthesized as a single polypeptide chain and then subjected to proteolysis to yield heterodimeric structures. PCT Publications Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional binding domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another).

The art has additionally noted the capability to produce diabodies that differ from such natural antibodies in being capable of binding two or more different epitope species (i.e., exhibiting bispecificity or multispecificity in addition to bivalency or multivalency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388/WO 02/02781 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng. Des. Sel. 17(1):21-27; Wu, A. et al. (2001) “Multimerization Of A Chimeric Anti-CD20 Single Chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T-Cell Engaging Antibodies For Cancer Therapy,” Cancer Res. 69(12):4941-4944).

The design of a diabody is based on the antibody derivative known as a single-chain Variable Domain fragment (scFv). Such molecules are made by linking Light and/or Heavy Chain Variable Domains using a short linking peptide. Bird et al. (1988) (“Single-Chain Antigen-Binding Proteins,” Science 242:423-426) describes example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen-Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The provision of bispecific binding molecules (e.g., non-monospecific diabodies) provides a significant advantage over antibodies, including but not limited to, a “trans” binding capability sufficient to co-ligate and/or co-localize different cells that express different epitopes and/or a “cis” binding capability sufficient to co-ligate and/or co-localize different molecules expressed by the same cell. Bispecific binding molecules (e.g., non-monospecific diabodies) thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221).

The ability to produce bispecific diabodies has led to their use (in “trans”) to co-ligate two cells together, for example, by co-ligating receptors that are present on the surface of different cells (e.g., cross-linking cytotoxic T-cells to tumor cells) (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305; Marvin et al. (2005) “Recombinant Approaches To IgG-Like Bispecific Antibodies,” Acta Pharmacol. Sin. 26:649-658). Alternatively, or additionally, bispecific diabodies can be used (in “cis”) to co-ligate molecules, such as receptors, etc., that are present on the surface of the same cell. Co-ligation of different cells and/or receptors is useful to modulation effector functions and/or immune cell signaling. However, the above advantages come at a salient cost. The formation of such non-monospecific diabodies requires the successful assembly of two or more distinct and different polypeptides (i.e., such formation requires that the diabodies be formed through the heterodimerization of different polypeptide chain species). This fact is in contrast to monospecific diabodies, which are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bispecific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-monospecific diabodies, termed DART® (Dual Affinity Re-Targeting Reagents) diabodies; see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538; and Sloan, D. D. et al. (2015) “Targeting HIV Reservoir in Infected CD4 T Cells by Dual-Affinity Re-targeting Molecules (DARTs) that Bind HIV Envelope and Recruit Cytotoxic T Cells,” PLoS Pathog. 11(11):e1005233. doi: 10.1371/journal.ppat.1005233; Al Hussaini, M. et al. (2015) “Targeting CD123 In AML Using A T-Cell Directed Dual-Affinity Re-Targeting (DARTR) Platform,” Blood pii: blood-2014-05-575704; Chichili, G. R. et al. (2015) “A CD3xCD123 Bispecific DART For Redirecting Host T Cells To Myelogenous Leukemia: Preclinical Activity And Safety In Nonhuman Primates,” Sci. Transl. Med. 7(289):289ra82; Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449). Such diabodies comprise two or more covalently complexed polypeptides and involve engineering one or more cysteine residues into each of the employed polypeptide species that permit disulfide bonds to form and thereby covalently bond one or more pairs of such polypeptide chains to one another. For example, the addition of a cysteine residue to the C-terminus of such constructs has been shown to allow disulfide bonding between the involved polypeptide chains, stabilizing the resulting diabody without interfering with the diabody's binding characteristics.

Many variations of such molecules have been described (see, e.g., United States Patent Publications No. 2015/0175697; 2014/0255407; 2014/0099318; 2013/0295121; 2010/0174053; 2009/0060910; 2007-0004909; European Patent Publication No. EP 2714079; EP 2601216; EP 2376109; EP 2158221; EP 1868650; and PCT Publications No. WO 2012/162068; WO 2012/018687; WO 2010/080538; WO 2006/113665) and are provided herein.

Alternative constructs are known in the art for applications where a tetravalent molecule is desirable but an Fc is not required including, but not limited to, tetravalent tandem antibodies, also referred to as “TandAbs” (see, e.g. United States Patent Publications Nos. 2005-0079170, 2007-0031436, 2010-0099853, 2011-020667 2013-0189263; European Patent Publication Nos. EP 1078004, EP 2371866, EP 2361936 and EP 1293514; PCT Publications Nos. WO 1999/057150, WO 2003/025018, and WO 2013/013700) which are formed by the homo-dimerization of two identical chains each possessing a VH1, VL2, VH2, and VL2 Domain.

### IV. Preferred PD-1×CTLA-4 Bispecific Molecules

One embodiment of the present invention relates to PD-1×CTLA-4 bispecific molecules that are capable of binding to a “first epitope” and a “second epitope,” such epitopes not being identical to one another. Such bispecific molecules comprise “VL1”/“VH1” domains that are capable of binding to the first epitope and “VL2”/“VH2” domains that are capable of binding to the second epitope. The notations “VL1” and “VH1” denote, respectively, the Light Chain Variable Domain and Heavy Chain Variable Domain that bind the “first” epitope of such bispecific molecules. Similarly, the notations “VL2” and “VH2” denote, respectively, the Light Chain Variable Domain and Heavy Chain Variable Domain that bind the “second” epitope of such bispecific molecules. It is irrelevant whether a particular epitope is designated as the first vs. the second epitope; such notations having relevance only with respect to the presence and orientation of domains of the polypeptide chains of the binding molecules of the present invention. In one embodiment, one of such epitopes is an epitope of human PD-1 and the other of such epitopes is an epitope of CTLA-4. In certain embodiments, a bispecific molecule comprises more than two epitope-binding sites. Such bispecific molecules will bind at least one epitope of PD-1 and at least one epitope of CTLA-4 and may further bind additional epitopes of PD-1 and/or additional epitopes of CTLA-4.

The present invention particularly relates to PD-1×CTLA-4 bispecific molecules (e.g., bispecific antibodies, bispecific diabodies, trivalent binding molecules, etc.) that possess epitope-binding fragments of antibodies that enable them to be able to coordinately bind to at least one epitope of PD-1 and at least on epitope of CTLA-4. Selection of the VL and VH Domains of the polypeptide domains of such molecules is coordinated such that the VL Domain and VH Domain of the same polypeptide chain are not capable of forming an epitope-binding site capable of binding either PD-1 or CTLA-4. Such selection is additionally coordinated so that polypeptides chains that make up such PD-1×CTLA-4 bispecific molecules assemble to form at least one functional antigen binding site that is specific for at least one epitope of PD-1 and at least one functional antigen binding site that is specific for at least one epitope of CTLA-4.

The present invention particularly relates to such PD-1×CTLA-4 bispecific molecules that exhibit an activity that is enhanced relative to such activity of two monospecific molecules one of which possesses the Heavy Chain Variable Domain and the Light Chain Variable Domain of the antibody that binds PD-1 and the other of which possesses the Heavy Chain Variable Domain and the Light Chain Variable Domain of the antibody that binds CTLA-4. Examples of such activity includes attenuating the activity of PD-1, attenuating the activity of CTLA-4, enhancing immune system activation, enhancing effector function, enhancing anti-tumor activity. As used herein, such attenuation of activity refers to a decrease of 10% or more, a decrease of 20% or more, a decrease of 50% or more, a decrease of 80% or more, or a decrease of 90% or more in a PD-1 and/or CTLA-4 inhibitory activity, or the complete elimination of such PD-1 and/or CTLA-4 inhibitory activity. As used herein, such enhancement of activity refers to an enhancement of 10% or more, an enhancement of 20% or more, an enhancement of 50% or more, an enhancement of 80% or more, or an enhancement of 90% or more in an immune system-activating activity mediated by or affected by the expression or presence of PD-1 and/or CTLA-4, relative to the activity exhibited by two monospecific molecules one of which possesses the Heavy Chain Variable Domain and the Light Chain Variable Domain of the antibody that binds PD-1 and the other of which possesses the Heavy Chain Variable Domain and the Light Chain Variable Domain of the antibody that binds CTLA-4. Examples of immune system-activating activity include, but are not limited to immune cell (e.g., T-lymphocyte, NK-cell) proliferation, immune cell production and/or release of cytokines, immune cell production and/or release of lytic molecules (e.g., granzyme, perforin, etc.), and/or immune cell expression of activation markers. Cytokines which are released upon activation of the immune system are known in the art and include, but are not limited to: IFNγ, IL-2, and TNFα, (see, e.g., Janeway, C. A. et al. 2011) IMMUNOBIOLOGY” 8th ed. Garland Science Publishing, NY; Banyer, J. L. (2000) “Cytokines in innate and adaptive immunity,” Rev Immunogenet. 2:359-373). Activation markers expressed by immune cells are known in the art and include, but are not limited to, CD69, CD25, and CD107a (see, e.g., Janeway, C. A. et al. (2011) IMMUNOBIOLOGY” 8th ed. Garland Science Publishing, NY; Shipkova, M. and Wieland, E. (2012) “Surface markers of lymphocyte activation and markers of cell proliferation,” Clin Chim Acta 413:1338-1349).

A. PD-1×CTLA-4 Bispecific Antibodies

The instant invention encompasses bispecific antibodies capable of simultaneously binding to PD-1 and CTLA-4. In some embodiments, the bispecific antibody capable of simultaneously binding to PD-1 and CTLA-4 is produced using any of the methods described in PCT Publications No. WO 1998/002463, WO 2005/070966, WO 2006/107786 WO 2007/024715, WO 2007/075270, WO 2006/107617, WO 2007/046893, WO 2007/146968, WO 2008/003103, WO 2008/003116, WO 2008/027236, WO 2008/024188, WO 2009/132876, WO 2009/018386, WO 2010/028797, WO2010028796, WO 2010/028795, WO 2010/108127, WO 2010/136172, WO 2011/086091, WO 2011/133886, WO 2012/009544, WO 2013/003652, WO 2013/070565, WO 2012/162583, WO 2012/156430, WO 2013/174873, and WO 2014/022540, each of which is hereby incorporated herein by reference in its entirety.

B. PD-1×CTLA-4 Bispecific Diabodies Lacking Fc Regions

One embodiment of the present invention relates to bispecific diabodies that comprise, and most preferably are composed of, a first polypeptide chain and a second polypeptide chain, whose sequences permit the polypeptide chains to covalently bind to each other to form a covalently associated diabody that is capable of simultaneously binding to PD-1 and to CTLA-4.

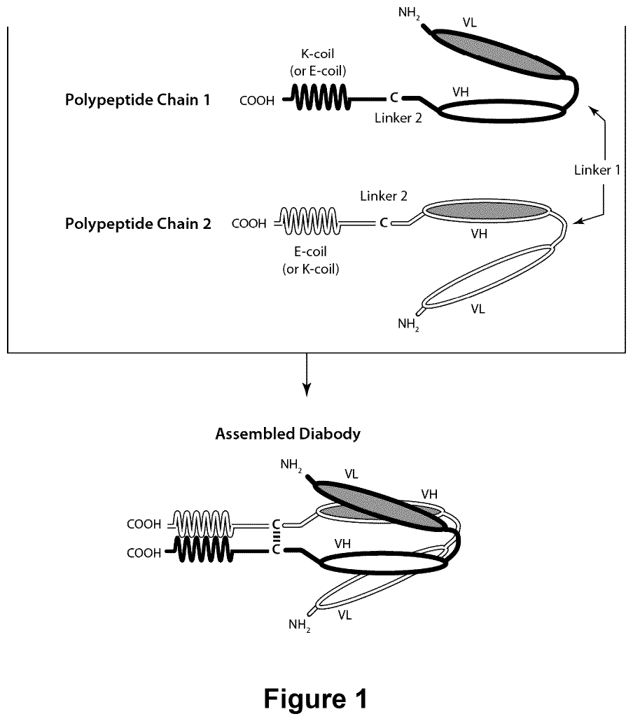

The first polypeptide chain of such an embodiment of bispecific diabodies comprises, in the N-terminal to C-terminal direction, an N-terminus, the VL Domain of a monoclonal antibody capable of binding to either PD-1 or CTLA-4 (i.e., either VL_{PD-1} or VL_{CTLA-4}), a first intervening spacer peptide (Linker 1), a VH Domain of a monoclonal antibody capable of binding to either CTLA-4 (if such first polypeptide chain contains VL_{PD-1}) or PD-1 (if such first polypeptide chain contains VL_{CTLA-4}), a second intervening spacer peptide (Linker 2) optionally containing a cysteine residue, a Heterodimer-Promoting Domain and a C-terminus (FIG. 1).

The second polypeptide chain of this embodiment of bispecific diabodies comprises, in the N-terminal to C-terminal direction, an N-terminus, a VL Domain of a monoclonal antibody capable of binding to either PD-1 or CTLA-4 (i.e., either VL_{PD-1} or VL_{CTLA-4}, and being the VL Domain not selected for inclusion in the first polypeptide chain of the diabody), an intervening spacer peptide (Linker 1), a VH Domain of a monoclonal antibody capable of binding to either CTLA-4 (if such second polypeptide chain contains VL_{PD-1}) or to PD-1 (if such second polypeptide chain contains VL_{CTLA-4}), a second intervening spacer peptide (Linker 2) optionally containing a cysteine residue, a Heterodimer-Promoting Domain, and a C-terminus (FIG. 1).

The VL Domain of the first polypeptide chain interacts with the VH Domain of the second polypeptide chain to form a first functional antigen-binding site that is specific for a first antigen (i.e., either PD-1 or CTLA-4). Likewise, the VL Domain of the second polypeptide chain interacts with the VH Domain of the first polypeptide chain in order to form a second functional antigen-binding site that is specific for a second antigen (i.e., either CTLA-4 or PD-1). Thus, the selection of the VL and VH Domains of the first and second polypeptide chains is coordinated, such that the two polypeptide chains of the diabody collectively comprise VL and VH Domains capable of binding to both an epitope of PD-1 and to an epitope of CTLA-4 (i.e., they collectively comprise VL_{PD-1}/VH_{PD-1} and VL_{CTLA-4}/VH_{CTLA-4}).

Most preferably, the length of the intervening linker peptide (Linker 1, which separates such VL and VH Domains) is selected to substantially or completely prevent the VL and VH Domains of the polypeptide chain from binding to one another (for example consisting of from 0, 1, 2, 3, 4, 5, 6, 7, 8 or 9 intervening linker amino acid residues). Thus the VL and VH Domains of the first polypeptide chain are substantially or completely incapable of binding to one another. Likewise, the VL and VH Domains of the second polypeptide chain are substantially or completely incapable of binding to one another. A preferred intervening spacer peptide (Linker 1) has the sequence (SEQ ID NO:5): GGGSGGGG.

The length and composition of the second intervening spacer peptide (Linker 2) is selected based on the choice of one or more polypeptide domains that promote such dimerization (i.e., a “Heterodimer-Promoting Domain”). Typically, the second intervening spacer peptide (Linker 2) will comprise 3-20 amino acid residues. In particular, where the employed Heterodimer-Promoting Domain(s) do/does not comprise a cysteine residue a cysteine-containing second intervening spacer peptide (Linker 2) is utilized. A cysteine-containing second intervening spacer peptide (Linker 2) will contain 1, 2, 3 or more cysteines. A preferred cysteine-containing spacer peptide (Linker 2) has the sequence is SEQ ID NO:6: GGCGGG. Alternatively, Linker 2 does not comprise a cysteine (e.g., GGG, GGGS (SEQ ID NO:7), LGGGSG (SEQ ID NO:8), GGGSGGGSGGG (SEQ ID NO:9), ASTKG (SEQ ID NO:10), LEPKSS (SEQ ID NO:11), APSSS (SEQ ID NO:12), etc.) and a Cysteine-Containing Heterodimer-Promoting Domain, as described below is used. Optionally, both a cysteine-containing Linker 2 and a cysteine-containing Heterodimer-Promoting Domain are used.

The Heterodimer-Promoting Domains may be GVEPKSC (SEQ ID NO:13) or VEPKSC (SEQ ID NO:14) or AEPKSC (SEQ ID NO:15) on one polypeptide chain and GFNRGEC (SEQ ID NO:16) or FNRGEC (SEQ ID NO:17) on the other polypeptide chain (US2007/0004909).

In a preferred embodiment, the Heterodimer-Promoting Domains will comprise tandemly repeated coil domains of opposing charge for example, “E-coil” helical domains (SEQ ID NO:18: EVAALEK-EVAALEK-EVAALEK-EVAALEK), whose glutamate residues will form a negative charge at pH 7, and “K-coil” domains (SEQ ID NO:19: KVAALKE-KVAALKE-KVAALKE-KVAALKE), whose lysine residues will form a positive charge at pH 7. The presence of such charged domains promotes association between the first and second polypeptides, and thus fosters heterodimer formation. Heterodimer-Promoting Domains that comprise modifications of the above-described E-coil and K-coil sequences so as to include one or more cysteine residues may be utilized. The presence of such cysteine residues permits the coil present on one polypeptide chain to become covalently bonded to a complementary coil present on another polypeptide chain, thereby covalently bonding the polypeptide chains to one another and increasing the stability of the diabody. Examples of such particularly preferred are Heterodimer-Promoting Domains include a Modified E-Coil having the amino acid sequence EVAACEK-EVAALEK-EVAALEK-EVAALEK (SEQ ID NO:20), and a modified K-coil having the amino acid sequence KVAACKE-KVAALKE-KVAALKE-KVAALKE (SEQ ID NO:21).

As disclosed in WO 2012/018687, in order to improve the in vivo pharmacokinetic properties of diabodies, a diabody may be modified to contain a polypeptide portion of a serum-binding protein at one or more of the termini of the diabody. Most preferably, such polypeptide portion of a serum-binding protein will be installed at the C-terminus of the diabody. Albumin is the most abundant protein in plasma and has a half-life of 19 days in humans. Albumin possesses several small molecule binding sites that permit it to non-covalently bind to other proteins and thereby extend their serum half-lives. The Albumin-Binding Domain 3 (ABD3) of protein G of Streptococcus strain G148 consists of 46 amino acid residues forming a stable three-helix bundle and has broad albumin-binding specificity (Johansson, M. U. et al. (2002) “Structure, Specificity, And Mode Of Interaction For Bacterial Albumin-Binding Modules,” J. Biol. Chem. 277(10):8114-8120. Thus, a particularly preferred polypeptide portion of a serum-binding protein for improving the in vivo pharmacokinetic properties of a diabody is the Albumin-Binding Domain (ABD) from streptococcal protein G, and more preferably, the Albumin-Binding Domain 3 (ABD3) of protein G of Streptococcus strain G148 (SEQ ID NO:22): LAEAKVLANR ELDKYGVSDY YKNLIDNAKS AEGVKALIDE ILAALP.

As disclosed in WO 2012/162068 (herein incorporated by reference), “deimmunized” variants of SEQ ID NO:22 have the ability to attenuate or eliminate MHC class II binding. Based on combinational mutation results, the following combinations of substitutions are considered to be preferred substitutions for forming such a deimmunized ABD: 66D/70S+71A; 66S/70S+71A; 66S/70S+79A; 64A/65A/71A; 64A/65A/71A+66S; 64A/65A/71A+66D; 64A/65A/71A+66E; 64A/65A/79A+66S; 64A/65A/79A+66D; 64A/65A/79A+66E. Variant ABDs having the modifications L64A, I65A and D79A or the modifications N66S, T70S and D79A. Variant deimmunized ABD having the amino acid sequence:

(SEQ ID NO: 23)
LAEAKVLANR ELDKYGVSDY YKNLID₆₆NAKS₇₀ A₇₁EGVKALIDE
ILAALP,

or the amino acid sequence:

(SEQ ID NO: 24)
LAEAKVLANR ELDKYGVSDY YKNA₆₄A₆₅NNAKT VEGVKALIA₇₉E
ILAALP,

or the amino acid sequence:

(SEQ ID NO: 25)
LAEAKVLANR ELDKYGVSDY YKNLIS₆₆NAKS₇₀
VEGVKALIA₇₉E ILAALP,

are particularly preferred as such deimmunized ABD exhibit substantially wild-type binding while providing attenuated MHC class II binding. Thus, the first polypeptide chain of such a diabody having an ABD contains a third linker (Linker 3) preferably positioned C-terminally to the E-coil (or K-coil) Domain of such polypeptide chain so as to intervene between the E-coil (or K-coil) Domain and the ABD (which is preferably a deimmunized ABD). A preferred sequence for such Linker 3 is SEQ ID NO:7: GGGS.

C. PD-1×CTLA-4 Bispecific Diabodies Containing Fc Regions

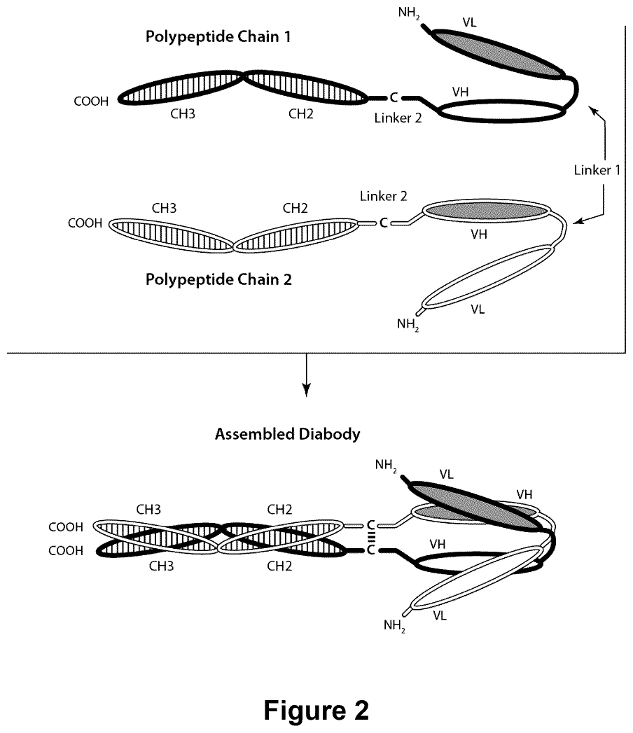

One embodiment of the present invention relates to bispecific diabodies capable of simultaneously binding to PD-1 and CTLA-4 that comprise an Fc Region. The addition of an IgG CH2-CH3 Domain to one or both of the diabody polypeptide chains, such that the complexing of the diabody chains results in the formation of an Fc Region, increases the biological half-life and/or alters the valency of the diabody. Incorporating an IgG CH2-CH3 Domains onto both of the diabody polypeptides will permit a two-chain bispecific Fc-Region-containing diabody to form (FIG. 2).

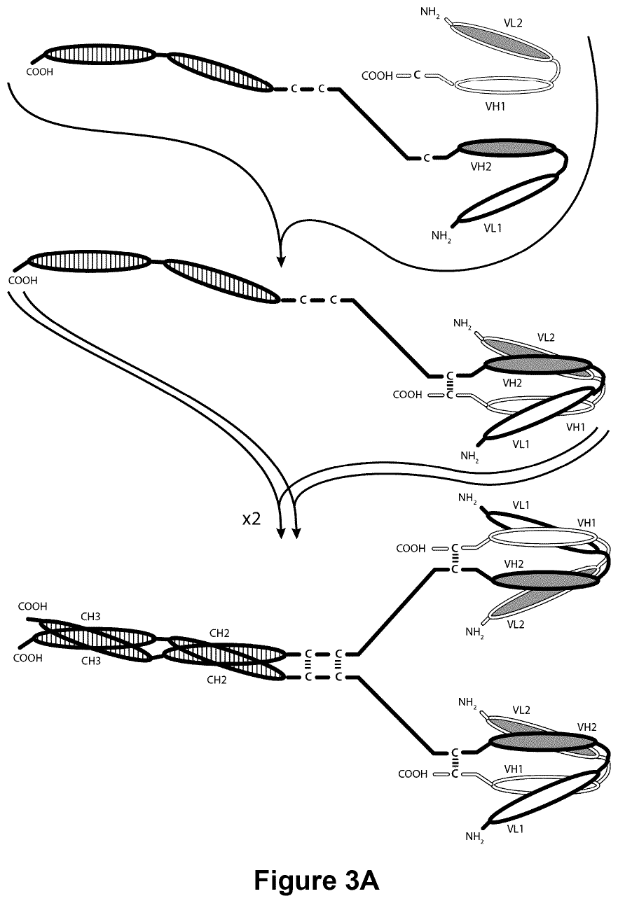

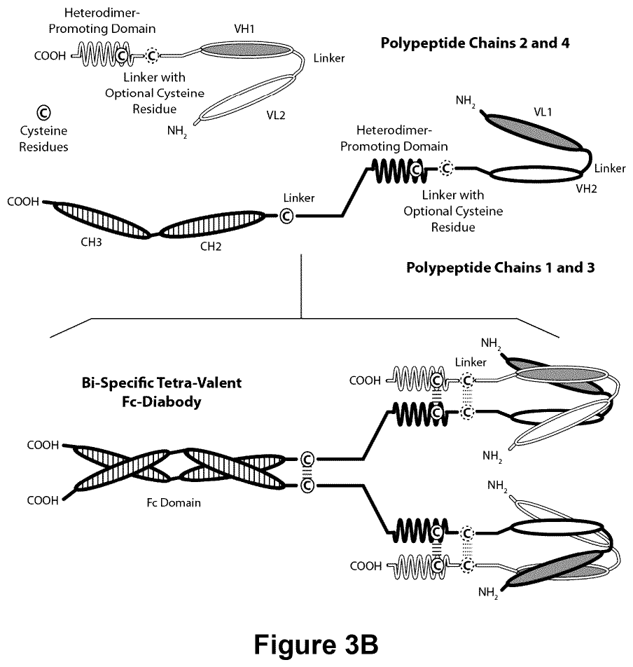

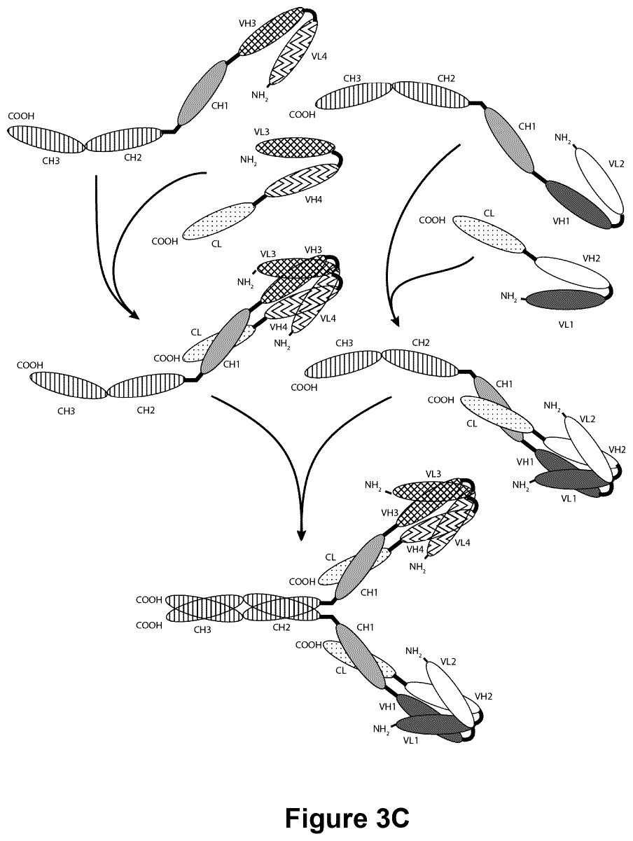

Alternatively, incorporating an IgG CH2-CH3 Domains onto only one of the diabody polypeptides will permit a more complex four-chain bispecific Fc Region-containing diabody to form (FIGS. 3A-3C). FIG. 3C shows a representative four-chain diabody possessing the Constant Light (CL) Domain and the Constant Heavy CH1 Domain, however fragments of such domains as well as other polypeptides may alternatively be employed (see, e.g., FIGS. 3A and 3B, United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538). Thus, for example, in lieu of the CH1 Domain, one may employ a peptide having the amino acid sequence GVEPKSC (SEQ ID NO:13) VEPKSC (SEQ ID NO:14), or AEPKSC (SEQ ID NO:15), derived from the Hinge Region of a human IgG, and in lieu of the CL Domain, one may employ the C-terminal 6 amino acids of the human kappa light chain, GFNRGEC (SEQ ID NO:16) or FNRGEC (SEQ ID NO:17). A representative peptide containing four-chain diabody is shown in FIG. 3A. Alternatively, or in addition, one may employ a peptide comprising tandem coil domains of opposing charge such as the “E-coil” helical domains (SEQ ID NO:18: EVAALEK-EVAAL^{EK-EVAALEK-EVAALEK or SEQ ID NO:}19: EVAACEK-EVAALEK-EVAALEK-EVAALEK); and the “K-coil” domains (SEQ ID NO:20: KVAALKE-KVAALKE-KVAALKE-KVAALKE or SEQ ID NO:21: KVAACKE-KVAALKE-KVAALKE-KVAALKE). A representative coil domain-containing four-chain diabody is shown in FIG. 3B.

The bispecific Fc Region-containing molecules of the present invention may include additional intervening spacer peptides (Linkers), generally such Linkers will be incorporated between a peptide Heterodimer-Promoting Domain (e.g., an E-coil or K-coil) and CH2-CH3 Domains and/or between CH2-CH3 Domains and a Variable Domain (i.e., VH or VL). Typically, the additional Linkers will comprise 3-20 amino acid residues. Linkers that may be employed in the bispecific Fc Region-containing diabody molecules of the present invention include: GGGS (SEQ ID NO:7), LGGGSG (SEQ ID NO:8), GGGSGGGSGGG (SEQ ID NO:9), ASTKG (SEQ ID NO:10), DKTHTCPPCP (SEQ ID NO:26), EPKSCDKTHTCPPCP (SEQ ID NO:27), LEPKSS (SEQ ID NO:11), APSSS (SEQ ID NO:28), and APSSSPME (SEQ ID NO:29), LEPKSADKTHTCPPC SEQ ID NO:30), GGC, and GGG. SEQ ID NO:11 may be used in lieu of GGG or GGC for ease of cloning. Additionally, the amino acids GGG, or SEQ ID NO:11 may be immediately followed by SEQ ID NO:26 to form the alternate linkers: GGGDKTHTCPPCP (SEQ ID NO:31); and LEPKSSDKTHTCPPCP (SEQ ID NO:32). Bispecific Fc Region-containing molecules of the present invention may incorporate an IgG Hinge Region in addition to or in place of a linker. Exemplary Hinge Regions include: EPKSCDKTHTCPPCP (SEQ ID NO:33) from IgG1, ERKCCVECPPCP (SEQ ID NO:34) from IgG2, ESKYGPPCPSCP (SEQ ID NO:35) from IgG4, and ESKYGPPCPPCP (SEQ ID NO:36) an IgG4 hinge variant comprising a stabilizing S228P substitution (as numbered by the EU index as set forth in Kabat) to reduce strand exchange.

As provided in FIG. 3A-3C, bispecific Fc Region-containing diabodies of the invention may comprise four different chains. The first and third polypeptide chains of such a diabody contain three domains: (i) a VL1-containing Domain, (ii) a VH2-containing Domain, (iii) Heterodimer-Promoting Domain and (iv) a Domain containing a CH2-CH3 sequence. The second and fourth polypeptide chains contain: (i) a VL2-containing Domain, (ii) a VH1-containing Domain and (iii) a Heterodimer-Promoting Domain, where the Heterodimer-Promoting Domains promote the dimerization of the first/third polypeptide chains with the second/fourth polypeptide chains. The VL and/or VH Domains of the third and fourth polypeptide chains, and VL and/or VH Domains of the first and second polypeptide chains may be the same or different so as to permit tetravalent binding that is either monospecific, bispecific or tetraspecific. The notations “VL3” and “VH3” denote, respectively, the Light Chain Variable Domain and Variable Heavy Chain Domain that bind a “third” epitope of such diabody. Similarly, the notations “VL4” and “VH4” denote, respectively, the Light Chain Variable Domain and Variable Heavy Chain Domain that bind a “fourth” epitope of such diabody. The general structure of the polypeptide chains of a representative four-chain bispecific Fc Region-containing diabodies of invention is provided in Table 1:

TABLE 1

| | | |
|---|---|---|
| Bi- | 2^{nd} Chain | NH₂—VL2—VH1—HPD—COOH |
| specific | 1^{st} Chain | NH₂—VL1—VH2—HPD—CH2—CH3—COOH |
| | 1^{st} Chain | NH₂—VL1—VH2—HPD—CH2—CH3—COOH |
| | 2^{nd} Chain | NH₂—VL2—VH1—HPD—COOH |
| Tetra- | 2^{nd} Chain | NH₂—VL2—VH1—HPD—COOH |
| specific | 1^{st} Chain | NH₂—VL1—VH2—HPD—CH2—CH3—COOH |
| | 3^{rd} Chain | NH₂—VL3—VH4—HPD—CH2—CH3—COOH |
| | 4^{th} Chain | NH₂—VL4—VH3—HPD—COOH |

HPD = Heterodimer-Promoting Domain

In a specific embodiment, diabodies of the present invention are bispecific, tetravalent (i.e., possess four epitope-binding sites), Fc-containing diabodies that are composed of four total polypeptide chains (FIGS. 3A-3C). The bispecific, tetravalent, Fc-containing diabodies of the invention comprise two epitope-binding sites immunospecific for PD-1 (which may be capable of binding to the same epitope of PD-1 or to different epitopes of PD-1), and two epitope-binding sites immunospecific for CTLA-4 (which may be capable of binding to the same epitope of CTLA-4 or to different epitopes of CTLA-4).

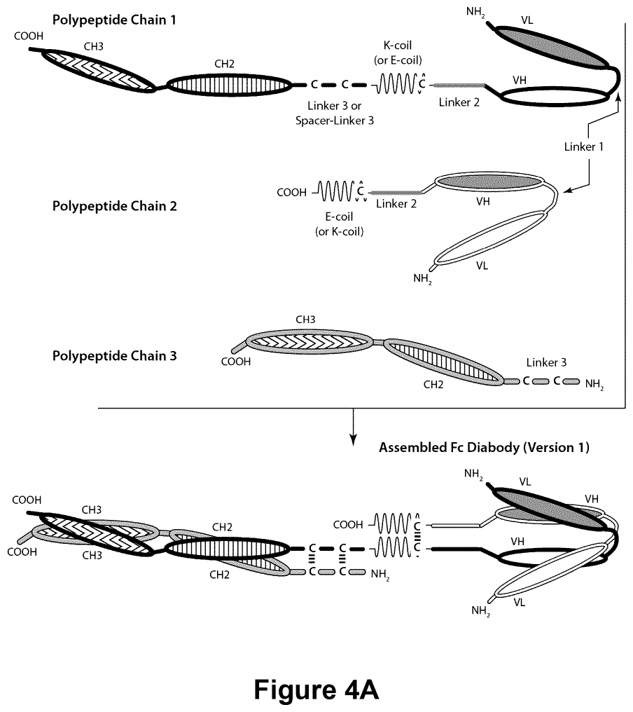

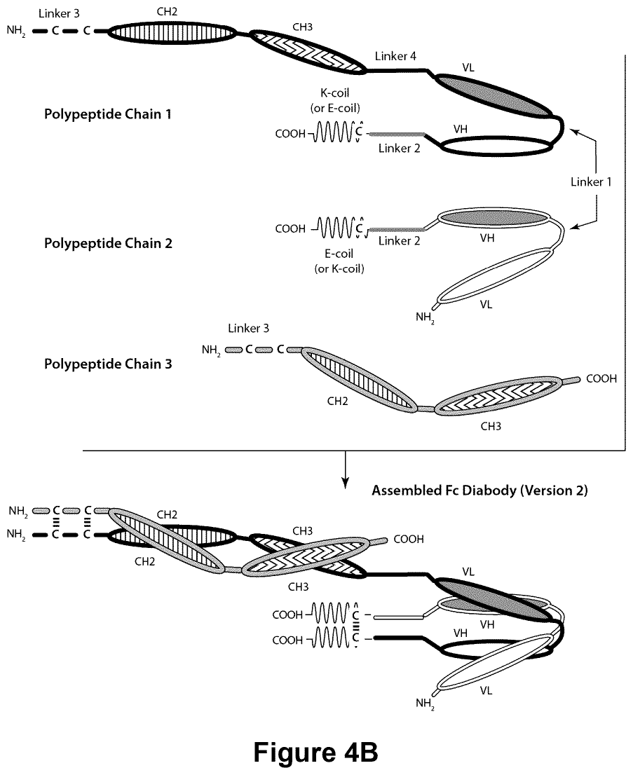

In a further embodiment, the bispecific Fc Region-containing diabodies may comprise three polypeptide chains. The first polypeptide of such a diabody contains three domains: (i) a VL1-containing Domain, (ii) a VH2-containing Domain and (iii) a Domain containing a CH2-CH3 sequence. The second polypeptide of such a diabody contains: (i) a VL2-containing Domain, (ii) a VH1-containing Domain and (iii) a Domain that promotes heterodimerization and covalent bonding with the diabody's first polypeptide chain. The third polypeptide of such a diabody comprises a CH2-CH3 sequence. Thus, the first and second polypeptide chains of such a diabody associate together to form a VL1/VH1 binding site that is capable of binding to the first epitope (i.e., either PD-1 or CTLA-4), as well as a VL2/VH2 binding site that is capable of binding to the second epitope (i.e., either CTLA-4 or PD-1). The first and second polypeptides are bonded to one another through a disulfide bond involving cysteine residues in their respective Third Domains. Notably, the first and third polypeptide chains complex with one another to form an Fc Region that is stabilized via a disulfide bond. Such bispecific diabodies have enhanced potency. FIGS. 4A and 4B illustrate the structures of such diabodies. Such Fc-Region-containing bispecific diabodies may have either of two orientations (Table 2):

TABLE 2

| | | |
|---|---|---|
| First | 3^{rd} Chain | NH₂—CH2—CH3—COOH |
| Orientation | 1^{st} Chain | NH₂—VL1—VH2—HPD—CH2—CH3—COOH |
| | 2^{nd} Chain | NH₂—VL2—VH1—HPD—COOH |
| Second | 3^{rd} Chain | NH₂—CH2—CH3—COOH |
| Orientation | 1^{st} Chain | NH₂—CH2—CH3—VL1—VH2—HPD—COOH |
| | 2^{nd} Chain | NH₂—VL2—VH1—HPD—COOH |

HPD = Heterodimer-Promoting Domain

In a specific embodiment, diabodies of the present invention are bispecific, bivalent (i.e., possess two epitope-binding sites), Fc-containing diabodies that are composed of three total polypeptide chains (FIGS. 4A-4B). The bispecific, bivalent Fc-containing diabodies of the invention comprise one epitope-binding site immunospecific for PD-1, and one epitope-binding site specific for CTLA-4.

In a further embodiment, the bispecific Fc Region-containing diabodies may comprise a total of five polypeptide chains. In a particular embodiment, two of the five polypeptide chains have the same amino acid sequence. The first polypeptide chain of such a diabody contains: (i) a VH1-containing domain, (ii) a CH1-containing domain, and (iii) a Domain containing a CH2-CH3 sequence. The first polypeptide chain may be the heavy chain of an antibody that contains a VH1 and a heavy chain constant region. The second and fifth polypeptide chains of such a diabody contain: (i) a VL1-containing domain, and (ii) a CL-containing domain. The second and/or fifth polypeptide chains of such a diabody may be light chains of an antibody that contains a VL1 complementary to the VH1 of the first/third polypeptide chain. The first, second and/or fifth polypeptide chains may be isolated from a naturally occurring antibody. Alternatively, they may be constructed recombinantly. The third polypeptide chain of such a diabody contains: (i) a VH1-containing domain, (ii) a CH1-containing domain, (iii) a Domain containing a CH2-CH3 sequence, (iv) a VL2-containing Domain, (v) a VH3-containing Domain and (vi) a Heterodimer-Promoting Domain, where the Heterodimer-Promoting Domains promote the dimerization of the third chain with the fourth chain. The fourth polypeptide of such diabodies contains: (i) a VL3-containing Domain, (ii) a VH2-containing Domain and (iii) a Domain that promotes heterodimerization and covalent bonding with the diabody's third polypeptide chain.

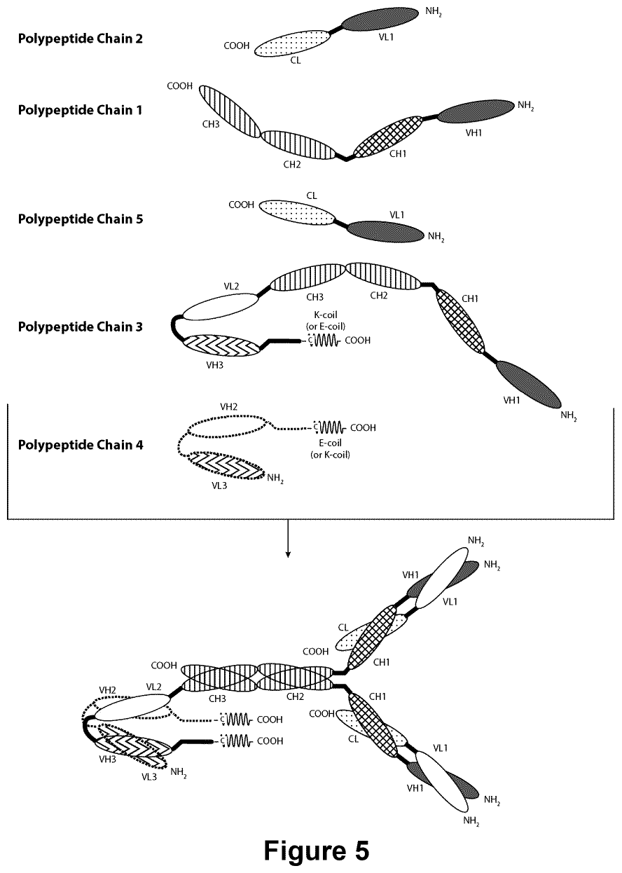

Thus, the first and second, and the third and fifth, polypeptide chains of such diabodies associate together to form two VL1/VH1 binding sites capable of binding a first epitope. The third and fourth polypeptide chains of such diabodies associate together to form a VL2/VH2 binding site that is capable of binding to a second epitope, as well as a VL3/VH3 binding site that is capable of binding to a third epitope. The first and third polypeptides are bonded to one another through a disulfide bond involving cysteine residues in their respective constant regions. Notably, the first and third polypeptide chains complex with one another to form an Fc Region. Such bispecific diabodies have enhanced potency. FIG. 5 illustrates the structure of such diabodies. It will be understood that the VL1/VH1, VL2/VH2, and VL3/VH3 Domains may be the same or different so as to permit binding that is monospecific, bispecific or trispecific. However, as provided herein, these domains are preferably selected so as to bind PD-1 and CTLA-4.

The VL and VH Domains of the polypeptide chains are selected so as to form VL/VH binding sites specific for a desired epitope. The VL/VH binding sites formed by the association of the polypeptide chains may be the same or different so as to permit tetravalent binding that is monospecific, bispecific, trispecific or tetraspecific. In particular, the VL and VH Domains may be selected such that a bispecific diabody may comprise two binding sites for a first epitope and two binding sites for a second epitope, or three binding sites for a first epitope and one binding site for a second epitope, or two binding sites for a first epitope, one binding site for a second epitope and one binding site for a third epitope (as depicted in FIG. 5). The general structure of the polypeptide chains of representative five-chain Fc Region-containing diabodies of invention is provided in Table 3:

TABLE 3

| | | |
|---|---|---|
| Bispecific | 2^{nd} Chain | NH₂—VL1—CL—COOH |
| (2 × 2) | 1^{st} Chain | NH₂—VH1—CH1—CH2—CH3—COOH |
| | 3^{rd} Chain | NH₂—VH1—CH1—CH2—CH3— |
| | | VL2—VH2—HPD—COOH |
| | 5^{nd} Chain | NH₂—VL1—CL—COOH |
| | 4^{th} Chain | NH₂—VL2—VH2—HPD—COOH |
| Bispecific | 2^{nd} Chain | NH₂—VL1—CL—COOH |
| (3 × 1) | 1^{st} Chain | NH₂—VH1—CH1—CH2—CH3—COOH |
| | 3^{rd} Chain | NH₂—VH1—CH1—CH2—CH3—VL1— |
| | | VH2—HPD—COOH |
| | 5^{nd} Chain | NH₂—VL1—CL—COOH |
| | 4^{th} Chain | NH₂—VL2—VH1—HPD—COOH |
| Trispecific | 2^{nd} Chain | NH₂—VL1—CL—COOH |
| (2 × 1 × 1) | 1^{st} Chain | NH₂—VH1—CH1—CH2—CH3—COOH |
| | 3^{rd} Chain | NH₂—VH1—CH1—CH2—CH3—VL2— |
| | | VH3—HPD—COOH |
| | 5^{nd} Chain | NH₂—VL1—CL—COOH |
| | 4^{th} Chain | NH₂—VL3—VH2—HPD—COOH |

HPD = Heterodimer-Promoting Domain

In a specific embodiment, diabodies of the present invention are bispecific, tetravalent (i.e., possess four epitope-binding sites), Fc-containing diabodies that are composed of five total polypeptide chains having two epitope-binding sites immunospecific for PD-1 (which may be capable of binding to the same epitope of PD-1 or to different epitopes of PD-1), and two epitope-binding sites specific for CTLA-4 (which may be capable of binding to the same epitope of CTLA-4 or to different epitopes of CTLA-4). In another embodiment, the bispecific, tetravalent, Fc-containing diabodies of the invention comprise three epitope-binding sites immunospecific for PD-1 (which may be capable of binding to the same epitope of PD-1 or to two or three different epitopes of PD-1), and one epitope-binding site specific for CTLA-4. In another embodiment, the bispecific, tetravalent, Fc-containing diabodies of the invention comprise one epitope-binding sites immunospecific for PD-1, and three epitope-binding sites specific for CTLA-4 (which may be capable of binding to the same epitope of CTLA-4 or to two or three different epitopes of CTLA-4).

D. PD-1×CTLA-4 Bispecific Trivalent Binding Molecules Containing Fc Regions

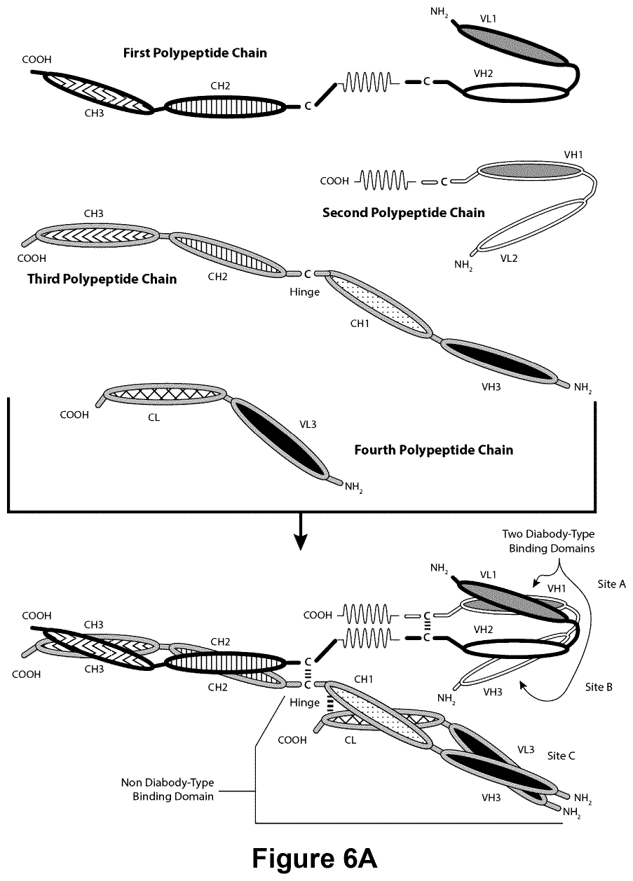

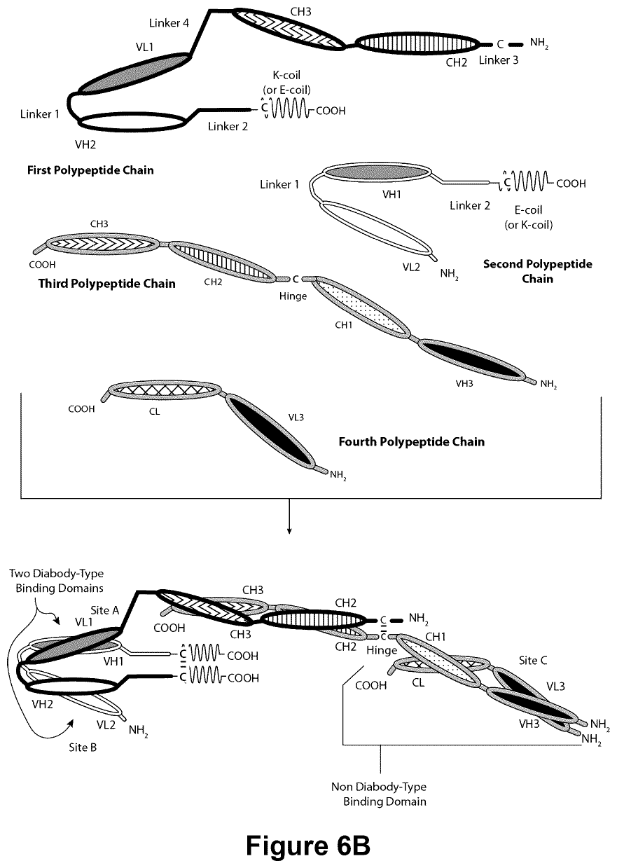

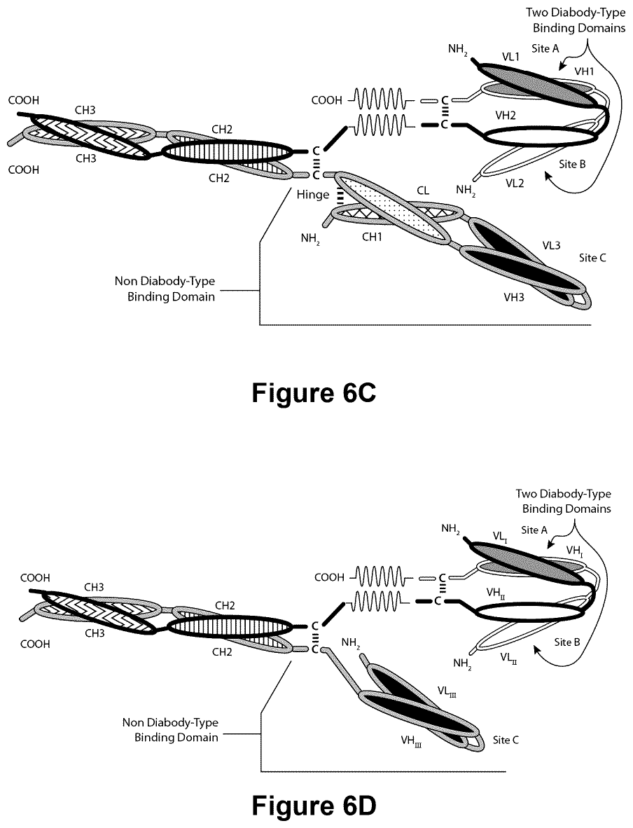

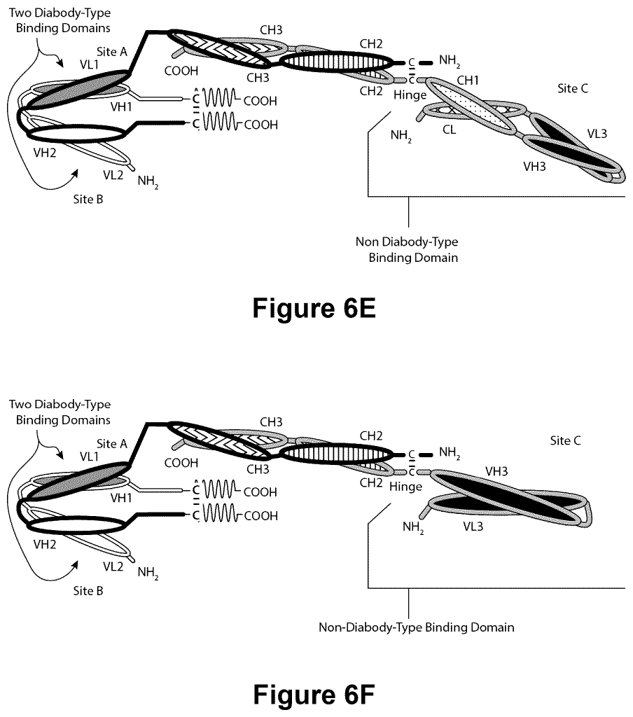

A further embodiment of the present invention relates to bispecific trivalent binding molecules comprising an Fc Region capable of simultaneously binding to an epitope of PD-1 and an epitope present on CTLA-4. Such bispecific trivalent binding molecules comprise three epitope-binding sites, two of which are Diabody-Type Binding Domains, which provide binding Site A and binding Site B, and one of which is a Fab-Type Binding Domain (or an scFv-Type Binding Domain), which provides binding Site C (see, e.g., FIGS. 6A-6F, and PCT Application No: PCT/US15/33081; and PCT/US15/33076). Such bispecific trivalent molecules thus comprise “VL1”/“VH1” domains that are capable of binding to the first epitope and “VL2”/“VH2” domains that are capable of binding to the second epitope and “VL3” and “VH3” domains that are capable of binding to the “third” epitope of such trivalent molecule. A “Diabody-Type Binding Domain” is the type of epitope-binding site present in a diabody, and especially, a DART® diabody, as described above. Each of a “Fab-Type Binding Domain” and an “scFv-Type Binding Domain” are epitope-binding sites that are formed by the interaction of the VL Domain of an immunoglobulin light chain and a complementing VH Domain of an immunoglobulin heavy chain. Fab-Type Binding Domains differ from Diabody-Type Binding Domains in that the two polypeptide chains that form a Fab-Type Binding Domain comprise only a single epitope-binding site, whereas the two polypeptide chains that form a Diabody-Type Binding Domain comprise at least two epitope-binding sites. Similarly, scFv-Type Binding Domains also differ from Diabody-Type Binding Domains in that they comprise only a single epitope-binding site. Thus, as used herein Fab-Type, and scFv-Type Binding Domains are distinct from Diabody-Type Binding Domains.

Typically, the trivalent binding molecules of the present invention will comprise four different polypeptide chains (see FIGS. 6A-6B), however, the molecules may comprise fewer or greater numbers of polypeptide chains, for example by fusing such polypeptide chains to one another (e.g., via a peptide bond) or by dividing such polypeptide chains to form additional polypeptide chains, or by associating fewer or additional polypeptide chains via disulfide bonds. FIGS. 6C-6F illustrate this aspect of the present invention by schematically depicting such molecules having three polypeptide chains. As provided in FIGS. 6A-6F, the trivalent binding molecules of the present invention may have alternative orientations in which the Diabody-Type Binding Domains are N-terminal (FIGS. 6A, 6C and 6D) or C-terminal (FIGS. 6B, 6E and 6F) to an Fc Region.

In certain embodiments, the first polypeptide chain of such trivalent binding molecules of the present invention contains: (i) a VL1-containing Domain, (ii) a VH2-containing Domain, (iii) a Heterodimer-Promoting Domain, and (iv) a Domain containing a CH2-CH3 sequence. The VL1 and VL2 Domains are located N-terminal or C-terminal to the CH2-CH3-containing domain as presented in Table 4 (also see, FIGS. 6A and 6B). The second polypeptide chain of such embodiments contains: (i) a VL2-containing Domain, (ii) a VH1-containing Domain, and (iii) a Heterodimer-Promoting Domain. The third polypeptide chain of such embodiments contains: (i) a VH3-containing Domain, (ii) a CH1-containing Domain and (iii) a Domain containing a CH2-CH3 sequence. The third polypeptide chain may be the heavy chain of an antibody that contains a VH3 and a heavy chain constant region, or a polypeptide that contains such domains. The fourth polypeptide of such embodiments contains: (i) a VL3-containing Domain and (ii) a CL-containing Domain. The fourth polypeptide chains may be a light chain of an antibody that contains a VL3 complementary to the VH3 of the third polypeptide chain, or a polypeptide that contains such domains. The third or fourth polypeptide chains may be isolated from naturally occurring antibodies. Alternatively, they may be constructed recombinantly, synthetically or by other means.

The Light Chain Variable Domain of the first and second polypeptide chains are separated from the Heavy Chain Variable Domains of such polypeptide chains by an intervening spacer peptide having a length that is too short to permit their VL1/VH2 (or their VL2/VH1) domains to associate together to form epitope-binding site capable of binding to either the first or second epitope. A preferred intervening spacer peptide (Linker 1) for this purpose has the sequence (SEQ ID NO:5): GGGSGGGG. Other Domains of the trivalent binding molecules may be separated by one or more intervening spacer peptides (Linkers), optionally comprising a cysteine residue. In particular, as provided above, such Linkers will typically be incorporated between Variable Domains (i.e., VH or VL) and peptide Heterodimer-Promoting Domains (e.g., an E-coil or K-coil) and between such peptide Heterodimer-Promoting Domains (e.g., an E-coil or K-coil) and CH2-CH3 Domains. Exemplary linkers useful for the generation of trivalent binding molecules are provided above and are also provided in PCT Application Nos: PCT/US15/33081; and PCT/US15/33076. Thus, the first and second polypeptide chains of such trivalent binding molecules associate together to form a VL1/VH1 binding site capable of binding a first epitope, as well as a VL2/VH2 binding site that is capable of binding to a second epitope. The third and fourth polypeptide chains of such trivalent binding molecules associate together to form a VL3/VH3 binding site that is capable of binding to a third epitope.

As described above, the trivalent binding molecules of the present invention may comprise three polypeptides. Trivalent binding molecules comprising three polypeptide chains may be obtained by linking the domains of the fourth polypeptide N-terminal to the VH3-containing Domain of the third polypeptide (e.g., using an intervening spacer peptide (Linker 4)). Alternatively, a third polypeptide chain of a trivalent binding molecule of the invention containing the following domains is utilized: (i) a VL3-containing Domain, (ii) a VH3-containing Domain, and (iii) a Domain containing a CH2-CH3 sequence, wherein the VL3 and VH3 are spaced apart from one another by an intervening spacer peptide that is sufficiently long (at least 9 or more amino acid residues) so as to allow the association of these domains to form an epitope-binding site. One preferred intervening spacer peptide for this purpose has the sequence: GGGGSGGGGSGGGGS (SEQ ID NO:37).

It will be understood that the VL1/VH1, VL2/VH2, and VL3/VH3 Domains of such trivalent binding molecules may be different so as to permit binding that is bispecific or trispecific. However, as provided herein, these domains are selected so as to provide a trivalent binding molecule capable of binding PD-1 and CTLA-4.

In particular, the VL and VH Domains may be selected such that a trivalent binding molecule comprises two binding sites for PD-1 (which may be capable of binding to the same epitope of PD-1 or to different epitopes of PD-1) and one binding sites for a CTLA-4, or one binding site for PD-1 and two binding sites for CTLA-4 (which may be capable of binding to the same epitope of CTLA-4 or to different epitopes of CTLA-4), or one binding site for PD-1, one binding site for CTLA-4 and one binding site for a third antigen that is not PD-1 or CTLA-4. The general structure of the polypeptide chains of representative trivalent binding molecules of invention is provided in FIGS. 6A-6F and in Table 4:

TABLE 4

| | | |
|---|---|---|
| Four Chain | 2^{nd} Chain | NH₂—VL2—VH1—HPD—COOH |
| 1^{st} | 1^{st} Chain | NH₂—VL1—VH2—HPD—CH2—CH3— |
| Orientation | | COOH |
| | 3^{rd} Chain | NH₂—VH3—CH1—CH2—CH3—COOH |
| | 2^{nd} Chain | NH₂—VL3—CL—COOH |
| Four Chain | 2^{nd} Chain | NH₂—VL2—VH1—HPD—COOH |
| 2nd | 1^{st} Chain | NH₂—CH2—CH3—VL1—VH2—HPD— |
| Orientation | | COOH |
| | 3^{rd} Chain | NH₂—VH3—CH1—CH2—CH3—COOH |
| | 2^{nd} Chain | NH₂—VL3—CL—COOH |
| | 2^{nd} Chain | NH₂—VL2—VH1—HPD—COOH |
| | 1^{st} Chain | NH₂—VL1—VH2—HPD—CH2—CH3— |
| | | COOH |
| Three | 3^{rd} Chain | NH₂—VL3—VH3—HPD—CH2—CH3— |
| Chain 1st | | COOH |
| Orientation | | |
| Three | 2^{nd} Chain | NH₂—VL2—VH1—HPD—COOH |
| Chain 2^{nd} | 1^{st} Chain | NH₂—CH2—CH3—VL1—VH2—HPD— |
| Orientation | | COOH |
| | 3^{rd} Chain | NH₂—VL3—VH3—HPD—CH2—CH3— |
| | | COOH |

HPD = Heterodimer-Promoting Domain

One embodiment of the present invention relates to bispecific trivalent binding molecules that comprise two epitope-binding sites for PD-1 and one epitope-binding site for CTLA-4.

The two epitope-binding sites for PD-1 may bind the same epitope or different epitopes. Another embodiment of the present invention relates to bispecific trivalent binding molecules that comprise, one epitope-binding site for PD-1 and two epitope-binding sites for CTLA-4. The two epitope-binding sites for CTLA-4 may bind the same epitope or different epitopes of CTLA-4. As provided above, such bispecific trivalent binding molecules may comprise three, four, five, or more polypeptide chains.

### V. Constant Domains and Fc Regions

Provided herein are antibody Constant Domains useful in the generation of the PD-1×CTLA-4 bispecific molecules (e.g., antibodies, diabodies, trivalent binding molecules, etc.) of the invention.

A preferred CL Domain is a human IgG CL Kappa Domain. The amino acid sequence of an exemplary human CL Kappa Domain is (SEQ ID NO:38):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG CL Lambda Domain. The amino acid sequence of an exemplary human CL Lambda Domain is (SEQ ID NO:39):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

As provided herein, the PD-1×CTLA-4 bispecific molecules of the invention may comprise an Fc Region. The Fc Region of such molecules of the invention may be of any isotype (e.g., IgG1, IgG2, IgG3, or IgG4). The PD-1×CTLA-4 bispecific molecules of the invention may further comprise a CH1 Domain and/or a Hinge Region. When present, the CH1 Domain and/or Hinge Region may be of any isotype (e.g., IgG1, IgG2, IgG3, or IgG4), and is preferably of the same isotype as the desired Fe Region.

An exemplary CH1 Domain is a human IgG1 CH Domain. The amino acid sequence of an exemplary human IgG1 CH1 Domain is (SEQ ID NO:40):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary CH1 Domain is a human IgG2 CH Domain. The amino acid sequence of an exemplary human IgG2 CH1 Domain is (SEQ ID NO:41):

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTV

An exemplary CH1 Domain is a human IgG4 CH1 Domain. The amino acid sequence of an exemplary human IgG4 CH1 Domain is (SEQ ID NO:42):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

One exemplary Hinge Region is a human IgG1 Hinge Region. The amino acid sequence of an exemplary human IgG1 Hinge Region is (SEQ ID NO:33):

EPKSCDKTHTCPPCP.

Another exemplary Hinge Region is a human IgG2 Hinge Region. The amino acid sequence of an exemplary human IgG2 Hinge Region is (SEQ ID NO:34): ERKCCVECPPCP.

Another exemplary Hinge Region is a human IgG4 Hinge Region. The amino acid sequence of an exemplary human IgG4 Hinge Region is (SEQ ID NO:35): ESKYGPPCPSCP. As described herein, an IgG4 Hinge Region may comprise a stabilizing mutation such as the S228P substitution. The amino acid sequence of an exemplary stabilized IgG4 Hinge Region is (SEQ ID NO:36): ESKYGPPCPPCP.

The Fc Region of the Fc Region-containing molecules (e.g., antibodies, diabodies, trivalent molecules, etc.) of the present invention may be either a complete Fc Region (e.g., a complete IgG Fc Region) or only a fragment of an Fc Region. Optionally, the Fc Region of the Fc Region-containing molecules of the present invention lacks the C-terminal lysine amino acid residue.

In traditional immune function, the interaction of antibody-antigen complexes with cells of the immune system results in a wide array of responses, ranging from effector functions such as antibody dependent cytotoxicity, mast cell degranulation, and phagocytosis to immunomodulatory signals such as regulating lymphocyte proliferation and antibody secretion. All of these interactions are initiated through the binding of the Fc Region of antibodies or immune complexes to specialized cell surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the neonatal Fc Receptor (FcRn) mediates the recycling of IgG molecules from the endosome to the cell surface and release into the blood. The amino acid sequence of exemplary wild-type IgG1 (SEQ ID NO:1), IgG2 (SEQ ID NO:2), IgG3 (SEQ ID NO:3), and IgG4 (SEQ ID NO:4) are presented above.

Modification of the Fc Region may lead to an altered phenotype, for example altered serum half-life, altered stability, altered susceptibility to cellular enzymes or altered effector function. It may therefore be desirable to modify an Fc Region-containing PD-1×CTLA-4 bispecific molecule of the present invention with respect to effector function, for example, so as to enhance the effectiveness of such molecule in treating cancer. Reduction or elimination of effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkin's lymphoma, CLL, and Burkitt's lymphoma). Molecules of the invention possessing such conferred or altered effector function activity are useful for the treatment and/or prevention of a disease, disorder or infection in which an enhanced efficacy of effector function activity is desired.

Accordingly, in certain embodiments, the Fc Region of the Fc Region-containing molecules of the present invention may be an engineered variant Fc Region. Although the Fc Region of the bispecific Fc Region-containing molecules of the present invention may possess the ability to bind to one or more Fc receptors (e.g., FcγR(s)), more preferably such variant Fc Region have altered binding to FcγRIA (CD64), FcγRIIA (CD32A), FcγRIIB (CD32B), FcγRIIIA (CD16a) or FcγRIIIB (CD16b) (relative to the binding exhibited by a wild-type Fe Region), e.g., will have enhanced binding to an activating receptor and/or will have substantially reduced or no ability to bind to inhibitory receptor(s). Thus, the Fc Region of the Fc Region-containing molecules of the present invention may include some or all of the CH2 Domain and/or some or all of the CH3 Domain of a complete Fc Region, or may comprise a variant CH2 and/or a variant CH3 sequence (that may include, for example, one or more insertions and/or one or more deletions with respect to the CH2 or CH3 domains of a complete Fc Region). Such Fc Regions may comprise non-Fc polypeptide portions, or may comprise portions of non-naturally complete Fc Regions, or may comprise non-naturally occurring orientations of CH2 and/or CH3 Domains (such as, for example, two CH2 domains or two CH3 domains, or in the N-terminal to C-terminal direction, a CH3 Domain linked to a CH2 Domain, etc.).

Fc Region modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890). Table 5 lists exemplary single, double, triple, quadruple and quintuple substitutions (relative to the amino acid sequence of SEQ ID NO:1) of exemplary modification that increase binding to activating receptors and/or reduce binding to inhibitory receptors.

TABLE 5
Variations of Preferred Activating Fc Regions
Single-Site Variations

| | | | |
|---|---|---|---|
| F243L | R292G | D270E | R292P |
| Y300L | P396L | | |

Double-Site Variations

| | | | |
|---|---|---|---|
| F243L and | F243L and | F243L and | R292P and |
| R292P | Y300L | P396L | Y300L |
| D270E and | R292P and | P396L and | P247L and |
| P396L | V305I | Q419H | N421K |
| R292P and | Y300L and | R255L and | R292P and |
| P396L | P396L | P396L | P305I |
| K392T and | | | |
| P396L | | | |

Triple-Site Variations

| | |
|---|---|
| F243L, P247L and N421K | P247L, D270E and N421K |
| F243L, R292P and Y300L | R255L, D270E and P396L |
| F243L, R292P and V305I | D270E, G316D and R416G |
| F243L, R292P and P396L | D270E, K392T and P396L |
| F243L, Y300L and P396L | D270E, P396L and Q419H |
| V284M, R292L and K370N | R292P, Y300L and P396L |

Quadruple-Site Variations

| | |
|---|---|
| L234F, F243L, R292P and Y300L | F243L, P247L, D270E and N421K |
| L234F, F243L, R292P and Y300L | F243L, R255L, D270E and P396L |
| L235I, F243L, R292P and Y300L | F243L, D270E, G316D and R416G |
| L235Q, F243L, R292P and Y300L | F243L, D270E, K392T and P396L |
| P247L, D270E, Y300L and N421K | F243L, R292P, Y300L, and P396L |
| R255L, D270E, R292G and P396L | F243L, R292P, V305I and P396L |
| R255L, D270E, Y300L and P396L | F243L, D270E, P396L and Q419H |
| D270E, G316D, P396L and R416G | |

Quintuple-Site Variations

| | |
|---|---|
| L235V, F243L, R292P, | F243L, R292P, V305I, |
| Y300L and P396L | Y300L and P396L |
| L235P, F243L, R292P, | |
| Y300L and P396L | |

Exemplary variants of human IgG1 Fe Regions with reduced binding to CD32B and/or increased binding to CD16A contain F243L, R292P, Y300L, V305I or P296L substitutions. These amino acid substitutions may be present in a human IgG1 Fc Region in any combination. In one embodiment, the human IgG1 Fc Region variant contains a F243L, R292P and Y300L substitution. In another embodiment, the human IgG1 Fc Region variant contains a F243L, R292P, Y300L, V305I and P296L substitution.

In certain embodiments, it is preferred for the Fc Regions of PD-1×CTLA-4 bispecific molecules of the present invention to exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type IgG1 Fc Region (SEQ ID NO: 1). In a specific embodiment, the PD-1×CTLA-4 bispecific molecules of the present invention comprise an IgG Fc Region that exhibits reduced ADCC effector function. In a preferred embodiment the CH2-CH3 Domains of such PD-1×CTLA-4 bispecific molecules include any 1, 2, 3, or 4 of the substitutions: L234A, L235A, D265A, N297Q, and N297G. In another embodiment, the CH2-CH3 Domains contain an N297Q substitution, an N297G substitution, L234A and L235A substitutions or a D265A substitution, as these mutations abolish FcR binding. Alternatively, a CH2-CH3 Domain of a naturally occurring Fc region that inherently exhibits decreased (or substantially no) binding to FcγRIIIA (CD16a) and/or reduced effector function (relative to the binding and effector function exhibited by the wild-type IgG1 Fe Region (SEQ ID NO:1)) is utilized. In a specific embodiment, the PD-1×CTLA-4 bispecific molecules of the present invention comprise an IgG2 Fc Region (SEQ ID NO:2) or an IgG4 Fc Region (SEQ ID:NO:4). When an IgG4 Fc Region is utilized, the instant invention also encompasses the introduction of a stabilizing mutation, such as the Hinge Region S228P substitution described above (see, e.g., SEQ ID NO:36). Since the N297G, N297Q, L234A, L235A and D265A substitutions abolish effector function, in circumstances in which effector function is desired, these substitutions would preferably not be employed.

A preferred IgG1 sequence for the CH2 and CH3 Domains of the Fc Region-containing molecules of the present invention having reduced or abolished effector function will comprise the substitutions L234A/L235A (SEQ ID NO:43):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein, X is a lysine (K) or is absent.

The serum half-life of proteins comprising Fc Regions may be increased by increasing the binding affinity of the Fc Region for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from the subject's body (e.g., human patient or other mammal) or a specific compartment thereof, for example, as measured in serum, i.e., circulating half-life, or in other tissues. In general, an increase in half-life results in an increase in mean residence time (MRT) in circulation for the molecule administered.

In some embodiments, the PD-1×CTLA-4 bispecific molecules of the present invention comprise a variant Fc Region, wherein the variant Fc Region comprises at least one amino acid modification relative to a wild-type Fc Region, such that the molecule has an increased half-life (relative to a molecule comprising a wild-type Fc Region). In some embodiments, the PD-1×CTLA-4 bispecific molecules of the present invention comprise a variant IgG Fc Region, wherein the variant Fc Region comprises a half-live extending amino acid substitution at one or more positions selected from the group consisting of 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436. Numerous mutations capable of increasing the half-life of an Fc Region-containing molecule are known in the art and include, for example M252Y, S254T, T256E, and combinations thereof. For example, see the mutations described in U.S. Pat. Nos. 6,277,375, 7,083,784; 7,217,797, 8,088,376; U.S. Publication Nos. 2002/0147311; 2007/0148164; and International Publication Nos. WO 98/23289; WO 2009/058492; and WO 2010/033279, which are herein incorporated by reference in their entireties. PD-1×CTLA-4 bispecific molecules with enhanced half-life also include those possessing variant Fc Regions comprising substitutions at two or more of Fc Region residues 250, 252, 254, 256, 257, 288, 307, 308, 309, 311, 378, 428, 433, 434, 435 and 436. In particular, two or more substitutions selected from: T250Q, M252Y, S254T, T256E, K288D, T307Q, V308P, A378V, M428L, N434A, H435K, and Y436I.

In a specific embodiment, a PD-1×CTLA-4 bispecific molecule possesses a variant IgG Fc Region comprising substitutions of:
(A) M252Y, S254T and T256E;
(B) M252Y and S254T;
(C) M252Y and T256E;
(D) T250Q and M428L;
(E) T307Q and N434A;
(F) A378V and N434A;
(G) N434A and Y436I;
(H) V308P and N434A; or
(I) K288D and H435K.

In a preferred embodiment PD-1×CTLA-4 bispecific molecules possess a variant IgG Fc Region comprising any 1, 2, or 3 of the substitutions: M252Y, S254T and T256E. The invention further encompasses PD-1×CTLA-4 bispecific molecules possessing variant Fc Regions comprising:
(A) one or more mutations which alter effector function and/or FcγR; and
(B) one or more mutations which extend serum half-life.

A preferred IgG1 sequence for the CH2 and CH3 Domains of the Fe Region-containing molecules of the present invention having increased serum half-life will comprise the substitutions M252Y, S254T and T256E (SEQ ID NO:80):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein, X is a lysine (K) or is absent.

As will be noted, the CH2-CH3 Domains of SEQ ID NO:80 includes substitutions at positions 234 and 235 with alanine, and thus form an Fc Region exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc Region (SEQ ID NO:1). The invention also encompasses such IgG1 CH2-CH3 Domains, which comprise the wild-type alanine residues, alternative and/or additional substitutions which modify effector function and/or F7R binding activity of the Fc region.

A preferred IgG4 sequence for the CH2 and CH3 Domains of the Fc Region-containing molecules of the present invention having increased serum half-life will comprise the substitutions M252Y, S254T and T256E (SEQ ID NO:81):

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLGX

wherein, X is a lysine (K) or is absent.

For certain antibodies, diabodies and trivalent binding molecules whose Fc Region-containing first and third polypeptide chains are not identical, it is desirable to reduce or prevent homodimerization from occurring between the CH2-CH3 Domains of two first polypeptide chains or between the CH2-CH3 Domains of two third polypeptide chains. The CH2 and/or CH3 Domains of such polypeptide chains need not be identical in sequence, and advantageously are modified to foster complexing between the two polypeptide chains. For example, an amino acid substitution (preferably a substitution with an amino acid comprising a bulky side group forming a “knob”, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., “the hole” (e.g., a substitution with glycine). Such sets of mutations can be engineered into any pair of polypeptides comprising CH2-CH3 Domains that forms an Fc Region to foster heterodimerization. Methods of protein engineering to favor heterodimerization over homodimerization are well known in the art, in particular with respect to the engineering of immunoglobulin-like molecules, and are encompassed herein (see e.g., Ridgway et al. (1996) “‘Knobs-Into-Holes’ Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety).

A preferred knob is created by modifying an IgG Fc Region to contain the modification T366W. A preferred hole is created by modifying an IgG Fc Region to contain the modification T366S, L368A and Y407V. To aid in purifying the hole-bearing third polypeptide chain homodimer from the final bispecific heterodimeric Fc Region-containing molecule, the protein A binding site of the hole-bearing CH2 and CH3 Domains of the third polypeptide chain is preferably mutated by amino acid substitution at position 435 (H435R). Thus, the hole-bearing third polypeptide chain homodimer will not bind to protein A, whereas the bispecific heterodimer will retain its ability to bind protein A via the protein A binding site on the first polypeptide chain. In an alternative embodiment, the hole-bearing third polypeptide chain may incorporate amino acid substitutions at positions 434 and 435 (N434A/N435K).

A preferred IgG1 amino acid sequence for the CH2 and CH3 Domains of the first polypeptide chain of an Fc Region-containing molecule of the present invention will have the “knob-bearing” sequence (SEQ ID NO:44):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent.

A preferred IgG1 amino acid sequence for the CH2 and CH3 Domains of the second polypeptide chain of an Fc Region-containing molecule of the present invention having two polypeptide chains (or the third polypeptide chain of an Fc Region-containing molecule having three, four, or five polypeptide chains) will have the “hole-bearing” sequence (SEQ ID NO:45):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is a lysine (K) or is absent.

As will be noted, the CH2-CH3 Domains of SEQ ID NO:44, and SEQ ID NO:45 include substitutions at positions 234 and 235 with alanine, and thus form an Fc Region exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc Region (SEQ ID NO:1). The invention also encompasses such IgG1 CH2-CH3 Domains, which comprise the wild-type alanine residues, alternative and/or additional substitutions which modify effector function and/or FγR binding activity of the Fc region. The invention also encompasses such CH2-CH3 Domains, which further comprise one or more half-live extending amino acid substitutions. In particular, as provided above, the invention encompasses such hole-bearing and such knob-bearing CH2-CH3 Domains which further comprise the M252Y/S254T/T256E.

A preferred IgG1 amino acid sequence, for the CH2 and CH3 Domains further comprising M252Y/S254T/T256E, of the first polypeptide chain of an Fc Region-containing molecule of the present invention will have the “knob-bearing” sequence (SEQ ID NO:82):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent.

A preferred IgG1 amino acid sequence, for the CH2 and CH3 Domains further comprising M252Y/S254T/T256E, of the second polypeptide chain of an Fc Region-containing molecule of the present invention having two polypeptide chains (or the third polypeptide chain of an Fe Region-containing molecule having three, four, or five polypeptide chains) will have the “hole-bearing” sequence (SEQ ID NO:83):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is a lysine (K) or is absent.

A preferred IgG4 amino acid sequence for the CH2 and CH3 Domains, comprising M252Y/S254T/T256E, of the first polypeptide chain of an Fc Region-containing molecule of the present invention will have the “knob-bearing” sequence (SEQ ID NO:84):

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLWCLVK GFYPSDIAVE WESNGQPENN
YKTTPPVLDS DGSFFLYSRL TVDKSRWQEG NVFSCSVMHE
ALHNHYTQKS LSLSLGX

wherein X is a lysine (K) or is absent.

A preferred IgG4 amino acid sequence, for the CH2 and CH3 Domains comprising M252Y/S254T/T256E, of the second polypeptide chain of an Fc Region-containing molecule of the present invention having two polypeptide chains (or the third polypeptide chain of an Fc Region-containing molecule having three, four, or five polypeptide chains) will have the “hole-bearing” sequence (SEQ ID NO:85):

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLSCAVK GFYPSDIAVE WESNGQPENN
YKTTPPVLDS DGSFFLVSRL TVDKSRWQEG NVFSCSVMHE
ALHNRYTQKS LSLSLGX

wherein X is a lysine (K) or is absent.

As will be noted, the CH2-CH3 Domains of SEQ ID NO:84, and SEQ ID NO:85 include the M252Y/S254T/T256E substitutions, and thus form an IgG4 Fc Region exhibiting increased serum half-life. The invention also encompasses IgG4 CH2-CH3 Domains, which comprise the wild-type M252/S254/T256 residues.

It is preferred that the first polypeptide chain will have a “knob-bearing” CH2-CH3 sequence, such as that of SEQ ID NO:44. However, as will be recognized, a “hole-bearing” CH2-CH3 Domain (e.g., SEQ ID NO:45) could be employed in the first polypeptide chain, in which case, a “knob-bearing” CH2-CH3 Domain (e.g., SEQ ID NO:44) would be employed in the second polypeptide chain of an Fc Region-containing molecule of the present invention having two polypeptide chains (or in the third polypeptide chain of an Fc Region-containing molecule having three, four, or five polypeptide chains).

In other embodiments, the invention encompasses PD-1×CTLA-4 bispecific molecules comprising CH2 and/or CH3 Domains that have been engineered to favor heterodimerization over homodimerization using mutations known in the art, such as those disclosed in PCT Publication No. WO 2007/110205; WO 2011/143545; WO 2012/058768; WO 2013/06867, all of which are incorporated herein by reference in their entirety.

### VI. Anti-PD-1 Binding Capabilities

Antibodies that are immunospecific for PD-1 are known (see, e.g., U.S. Patent Applications No. 62/198,867; 62/239,559; 62/255,140 U.S. Pat. Nos. 8,008,449; 8,552,154; PCT Patent Publications WO 2012/135408; WO 2012/145549; and WO 2013/014668). Preferred PD-1 binding capabilities useful in the generation of the PD-1×CTLA-4 bispecific molecules of the present invention are capable of binding to a continuous or discontinuous (e.g., conformational) portion (epitope) of human PD-1 (CD279) and will preferably also exhibit the ability to bind to PD-1 molecules of one or more non-human species, in particular, primate species (and especially a primate species, such as cynomolgus monkey). Additional desired antibodies may be made by isolating antibody-secreting hybridomas elicited using PD-1 or a peptide fragment thereof. A representative human PD-1 polypeptide (NCBI Sequence NP_005009.2; including a 20 amino acid residue signal sequence, shown underlined) and the 268 amino acid residue mature protein) has the amino acid sequence (SEQ ID NO:46):

MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQTLV VGVVGGLLGS LVLLVWVLAV ICSRAARGTI
GARRTGQPLK EDPSAVPVFS VDYGELDFQW REKTPEPPVP
CVPEQTEYAT IVFPSGMGTS SPARRGSADG PRSAQPLRPE
DGHCSWPL

Preferred anti-PD-1 binding molecules (e.g., antibodies) useful in the generation of the PD-1×CTLA-4 bispecific molecules of the instant invention possess the VL and/or VH Domains of the anti-human PD-1 monoclonal antibody “PD-1 mAb 1” (nivolumab, CAS Reg. No.:946414-94-4, also known as 5C4, BMS-936558, ONO-4538, MDX-1106, and marketed as OPDIVO® by Bristol-Myers Squibb); “PD-1 mAb 2” (pembrolizumab, (formerly known as lambrolizumab), CAS Reg. No.:1374853-91-4, also known as MK-3475, SCH-900475, and marketed as KEYTRUDA® by Merck); “PD-1 mAb 3” (EH12.2H7; Dana Farber), “PD-1 mAb 4” (pidilizumab, CAS Reg. No.: 1036730-42-3 also known as CT-011, CureTech), or any of the anti-PD-1 antibodies in Table 6; and more preferably possess 1, 2 or all 3 of the CDR_{L}s of the VL Region and/or 1, 2 or all 3 of the CDR_{H}s of the VH Domain of such anti-PD-1 monoclonal antibodies. Additional anti-PD-1 antibodies possessing unique binding characteristics useful in the methods and compositions of the instant inventions have recently been identified (see, United States Patent Application Nos. 62/198,867; 62/239,559; 62/255,140). Particularly, preferred are PD-1-binding molecules which possess a humanized VH and/or VL Domain of the anti-PD-1 antibody “PD-1 mAb 5” (hPD-1 mAb 2, MacroGenics); “PD-1 mAb 6” (hPD-1 mAb 7, MacroGenics); “PD-1 mAb 7” (hPD-1 mAb 9, MacroGenics); or “PD-1 mAb 8” (hPD-1 mAb 15, MacroGenics); and more preferably possess 1, 2 or all 3 of the CDR_{L}s of the VL Region and/or 1, 2 or all 3 of the CDR_{H}s of the VH Domain of such humanized anti-PD-1 monoclonal antibodies.

A. PD-1 mAb 1

The amino acid sequence of the VH Domain of PD-1 mAb 1 (SEQ ID NO:47) is shown below (CDR_{H} residues are shown underlined).

QVQLVESGGG VVQPGRSLRL DCKASGITFS NSGMHWVRQA
PGKGLEWVAV IWYDGSKRYY ADSVKGRFTI SRDNSKNTLF
LQMNSLRAED TAVYYCATND DYWGQGTLVT VSS

The amino acid sequence of the VL Domain of PD-1 mAb 1 (SEQ ID NO:48) is shown below (CDR_{L} residues are shown underlined).

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ SSNWPRTFGQ GTKVEIK

B. PD-1 mAb 2

The amino acid sequence of the VH Domain of PD-1 mAb 2 (SEQ ID NO:49) is shown below (CDR_{H} residues are shown underlined).

QVQLVQSGVE VKKPGASVKV SCKASGYTFT NYYMYWVRQA
PGQGLEWMGG INPSNGGTNF NEKFKNRVTL TTDSSITTAY
MELKSLQFDD TAVYYCARRD YRFDMGFDYW GQGTTVTVSS

The amino acid sequence of the VL Domain of PD-1 mAb 2 (SEQ ID NO:50) is shown below (CDR_{L} residues are shown underlined).

EIVLTQSPAT LSLSPGERAT LSCRASKGVS TSGYSYLHWY
QQKPGQAPRL LIYLASYLES GVPARFSGSG SGTDFTLTIS
SLEPEDFAVY YCQHSRDLPL TFGGGTKVEIK

C. PD-1 mAb 3

The amino acid sequence of the VH Domain of PD-1 mAb 3 (SEQ ID NO:51) is shown below (CDR_{H} residues are shown underlined).

QVQLQQSGAE LAKPGASVQM SCKASGYSFT SSWIHWVKQR
PGQGLEWIGY IYPSTGFTEY NQKFKDKATL TADKSSSTAY
MQLSSLTSED SAVYYCARWR DSSGYHAMDY WGQGTSVTVSS

The amino acid sequence of the VL Domain of PD-1 mAb 3 (SEQ ID NO:52) is shown below (CDR_{L} residues are shown underlined).

DIVLTQSPAS LTVSLGQRAT ISCRASQSVS TSGYSYMHWY
QQKPGQPPKL LIKFGSNLES GIPARFSGSG SGTDFTLNIH
PVEEEDTATY YCQHSWEIPY TFGGGTKLEI K

D. PD-1 mAb 4

The amino acid sequence of the VH Domain of PD-1 mAb 4 (SEQ ID NO:53) is shown below (CDR_{H} residues are shown underlined).

QVQLVQSGSE LKKPGASVKI SCKASGYTFT NYGMNWVRQA
PGQGLQWMGW INTDSGESTY AEEFKGRFVF SLDTSVNTAY
LQITSLTAED TGMYFCVRVG YDALDYWGQG TLVTVSS

The amino acid sequence of the VL Domain of PD-1 mAb 4 (SEQ ID NO:54) is shown below (CDR_{L} residues are shown underlined).

EIVLTQSPSS LSASVGDRVT ITCSARSSVS YMHWFQQKPG
KAPKLWIYRT SNLASGVPSR FSGSGSGTSY CLTINSLQPE
DFATYYCQQR SSFPLTFGGG TKLEIK

E. PD-1 mAb 5

The amino acid sequence of the VH Domain of PD-1 mAb 5 (SEQ ID NO:55) is shown below (CDR_{H} residues are shown underlined).

EVQLVESGGG LVQPGGSLRL SCAASGFVFS SFGMHWVRQA
PGKGLEWVAY ISSGSMSISY ADTVKGRFTI SRDNAKNTLY
LQMNSLRTED TALYYCASLS DYFDYWGQGT TVTVSS

The amino acid sequence of the VL Domain of PD-1 mAb 5 (SEQ ID NO:56) is shown below (CDR_{L} residues are shown underlined).

DVVMTQSPLS LPVTLGQPAS ISCRSSQSLV HSTGNTYLHW
YLQKPGQSPQ LLIYRVSNRF SGVPDRFSGS GSGTDFTLKI
SRVEAEDVGV YYCSQTTHVP WTFGQGTKLE IK

F. PD-1 mAb 6

The amino acid sequence of the VH Domain of PD-1 mAb 6 (SEQ ID NO:57) is shown below (CDR_{H} residues are shown underlined).

QVQLVQSGAE VKKPGASVKV SCKASGYSFT SYWMNWVRQA
PGQGLEWX₁GV IHPSDSETWL DQKFKDRVTI TVDKSTSTAY
MELSSLRSED TAVYYCAREH YGTSPFAYWG QGTLVTVSS

wherein X₁ is I or A

The amino acid sequence of the VL Domain of PD-1 mAb 6 (SEQ ID NO:58) is shown below (CDR_{L} residues are shown underlined).

EIVLTQSPAT LSLSPGERAT LSCRAX₁ESVD NYGMSFMNWF
QQKPGQPPKL LIHAASNX₂GS GVPSRFSGSG SGTDFTLTIS
SLEPEDFAVY FCQQSKEVPY TFGGGTKVEI K

wherein:
   X₁ is N or S and X₂ is Q or R; or
   X₁ is N and X₂ is Q; or
   X₁ is S and X₂ is Q; or
   X₁ is S and X₂ is R

In particular embodiments the amino acid sequence of PD-1 mAb 6 comprises:
(a) SEQ ID NO:57, wherein X₁ is I; and SEQ ID NO:58, wherein X₁ is N and X₂ is Q; or
(b) SEQ ID NO:57, wherein X₁ is I; and SEQ ID NO:58, wherein X₁ is S and X₂ is Q.

An exemplary anti-PD-1 VH Domain designated “PD-1 mAb 6-I VH” comprises SEQ ID NO:57 wherein X₁ is I and has the amino acid sequence (SEQ ID NO:86):

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSS

An exemplary anti-PD-1 VL Domain designated “PD-1 mAb 6-SQ VL” comprises SEQ ID NO:58 wherein X₁ is S and X₂ is Q and has the amino acid sequence (SEQ ID NO:87):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI K

An exemplary anti-PD-1 antibody that possesses a PD-1 mAb 6-I VH domain and a PD-1 mAb 6-SQ VL domain is designated as “PD-1 mAb 6-ISQ.”

G. PD-1 mAb 7

The amino acid sequence of the VH Domain of PD-1 mAb 7 (SEQ ID NO:59) is shown below (CDR_{H} residues are shown underlined).

EVQLVESGGG LX₁RPGGSLKL SCAASGFTFS SYLVX₂WVRQA
PGKGLEWX₃AT ISGGGGNTYY SDSVKGRFTI SRDNAKNSLY
LQMNSX₄RAED TATYYCARYG FDGAWFAYWG QGTLVTVSS

wherein:
   X₁ is V or A; X₂ is S or G; X₃ is V or T; X₄ is L or A; or
   X₁ is V, X₂ is S, X₃ is V, and X₄ is L; or
   X₁ is A, X₂ is G, X₃ is T, and X₄ is A

The amino acid sequence of the VL Domain of PD-1 mAb 7 (SEQ ID NO:60) is shown below (CDR_{L} residues are shown underlined).

DIQMTQSPSS LSASVGDRVT ITCRASENIY X₁YLAWYQQKP
GKAPKLLIYX₂ AKTLAAGVPS RFSGSGSGTD FTLTISSLQP
EDFATYYCQH HYAVPWTFGQ GTKLEIK

wherein:
   X₁ is S or N and X₂ is N or D; or X₁ is S and X₂ is N; or
   X₁ is N and X₂ is D

In particular embodiments PD-1 mAb 7 comprises:
(a) SEQ ID NO:59, wherein X₁ is V, X₂ is S, X₃ is V, and X₄ is L; and
   SEQ ID NO:60, wherein X₁ is S and X₂ is N; or
(b) SEQ ID NO:59, wherein X₁ is A, X₂ is G, X₃ is T, and X₄ is A; and
   SEQ ID NO:60, wherein X₁ is N and X₂ is D.

H. PD-1 mAb 8

The amino acid sequence of the VH Domain of PD-mAb 8 (SEQ ID NO:61) is shown below (CDR_{H} residues are shown underlined).

EVQLVESGGG LVRPGGSLRL SCAASGFTFS SYLISWVRQA
PGKGLEWVAA ISGGGADTYY ADSVKGRFTI SRDNAKNSLY
LQMNSLRAED TATYYCARRG TYAMDYWGQG TLVTVSS

The amino acid sequence of the VL Domain of PD-1 mAb 8 (SEQ ID NO:62) is shown below (CDR_{L} residues are shown underlined).

DIQMTQSPSS LSASVGDRVT ITCRASENIY NYLAWYQQKP
GKAPKLLIYD AKTLAAGVPS RFSGSGSGTD FTLTISSLQP
EDFATYYCQH HYAVPWTFGQ GTKLEIK

L. Additional Anti-PD-1 Antibodies

Additional anti-PD-1 antibodies which may be utilized to generate the PD-1×CTLA-4 bispecific molecules of the instant invention are provided in Table 6.

TABLE 6
Additional Anti-PD-1 Antibodies

| PD-1 Antibodies | Reference/Source |
|---|---|
| PD1-17; PD1-28; PD1-33; PD1-35; | U.S. Pat. Nos. 7,488,802; |
| and PD1-F2 | 7,521,051; and 8,088,905; PCT |
| | Patent Publication WO |
| | 2004/056875 |
| 17D8; 2D3; 4H1; 5C4; 4A11; 7D3; | U.S. Pat. Nos. 8,008,449; |
| and 5F4 | 8,779,105; and 9,084,776; PCT |
| | Patent Publication WO |
| | 2006/121168 |
| hPD-1.08A; hPD-1.09A; 109A; K09A; | U.S. Pat. Nos. 8,354,509; |
| 409A; h409A11; h409A16; h409A17; | 8,900,587; and 5,952,136; PCT |
| Codon optimized 109A; and Codon | Patent Publication WO |
| optimized 409A | 2008/156712 |
| 1E3; 1E8; and 1H3 | U.S. Patent Publication |
| | 2014/0044738; PCT Patent |
| | Publication WO 2012/145493 |
| 9A2; 10B11; 6E9; APE1922; APE1923; | PCT Patent Publication WO |
| APE1924; APE1950; APE1963; and | 2014/179664 |
| APE2058 | |
| GA1; GA2; GB1; GB6; GH1; A2; C7; H7; | U.S. Patent Publication |
| SH-A4; SH-A9; RG1H10; RG1H11; | 2014/0356363; PCT Patent |
| RG2H7; RG2H10; RG3E12; RG4A6; | Publication WO 2014/194302 |
| RG5D9; RG1H10-H2A-22-1S; RG1H10- | |
| H2A-27-2S; RG1H10-3C; RG1H10-16C; | |
| RG1H10-17C; RG1H10-19C; | |
| RG1H10-21C; and RG1H10-23C2 | |
| H1M7789N; H1M7799N; H1M7800N; | U.S. Patent Publication |
| H2M7780N; H2M7788N; H2M7790N; | 2015/0203579; PCT Patent |
| H2M7791N; H2M7794N; H2M7795N; | Publication WO 2015/112800 |
| H2M7796N; H2M7798N; H4H9019P; | |
| H4xH9034P2; H4xH9035P2; H4xH9037P2; | |
| H4xH9045P2; H4xH9048P2; H4H9057P2; | |
| H4H9068P2; H4xH9119P2; H4xH9120P2; | |
| H4Xh9128p2; H4Xh9135p2; H4Xh9145p2; | |
| H4Xh8992p; H4Xh8999p; and H4Xh9008p; | |
| PD-1 mAb 1; PD-1 mAb 2; hPD-1 mAb 2; | U.S. Patent Applications No. |
| PD-1 mAb 3; PD-1 mAb 4; PD-1 mAb 5; | 62/198,867 and 62/239,559 |
| PD-1 mAb 6; PD-1 mAb 7; hPD-1 mAb | |
| 7; PD-1 mAb 8; PD-1 mAb 9; hPD-1 mAb | |
| 9; PD-1 mAb 10; PD-1 mAb 11; PD-1 | |
| mAb 12; PD-1 mAb 13; PD-1 mAb 14; | |
| PD-1 mAb 15; and hPD-1 mAb 15 | |

J. Exemplary Anti-PD-1 Antibody

An exemplary anti-PD-1 antibody designated “PD-1 mAb 6 G4P” comprises: a heavy chain having the VH Domain of PD-1 mAb 61 (SEQ ID NO:86), an IgG4 CH1 Domain (SEQ ID NO:42), a stabilized IgG 4 Hinge (SEQ ID NO:36), and IgG4 CH2-CH3 Domains lacking the C-terminal lysine (SEQ ID NO:4); and a light chain having the VL Domain of PD-1 mAb 6SQ (SEQ ID NO:87) and a kappa CL (SEQ ID NO:38).

The amino acid sequence of the complete heavy chain of PD-1 mAb 6 G4P (SEQ ID NO:88) is shown below.

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSSA
STKGPSVFPL APCSRSTSES TAALGCLVKD YFPEPVTVSW
NSGALTSGVH TFPAVLQSSG LYSLSSVVTV PSSSLGTKTY
TCNVDHKPSN TKVDKRVESK YGPPCPPCPA PEFLGGPSVF
LFPPKPKDTL MISRTPEVTC VVVDVSQEDP EVQFNWYVDG
VEVHNAKTKP REEQFNSTYR VVSVLTVLHQ DWLNGKEYKC
KVSNKGLPSS IEKTISKAKG QPREPQVYTL PPSQEEMTKN
QVSLTCLVKG FYPSDIAVEW ESNGQPENNY KTTPPVLDSD
GSFFLYSRLT VDKSRWQEGN VFSCSVMHEA LHNHYTQKSL
SLSLG

The amino acid sequence of the complete light chain of PD-1 mAb 6 G4P (SEQ ID NO:89) is shown below.

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

### VI. Anti-CTLA-4 Binding Capabilities

Antibodies that are immunospecific for CTLA-4 are known (see, e.g., U.S. Pat. Nos. 6,984,720; 6,682,736; 7,034,121; 7,109,003; 7,132,281; 7,411,057; 7,605,238; 7,807,797; 7,824,679; 8,017,114; 8,143,379; 8,318,916; 8,491,895; 8,784,815; and 8,883,984; US Patent Publications 2009/0123477; 2009/0252741; and 2014/0105914; PCT Patent Publications No. WO 00/37504; WO 01/14424; WO 01/54732; WO 2006/029219; WO 2006/066568; and WO 2012/120125; and Table 7). Preferred CTLA-4 binding capabilities useful in the generation of the PD-1×CTLA-4 bispecific molecules of the present invention are capable of binding to a continuous or discontinuous (e.g., conformational) portion (epitope) of human CTLA-4 and will preferably also exhibit the ability to bind to CTLA-4 molecules of one or more non-human species, in particular, primate species (and especially a primate species, such as cynomolgus monkey). Additional desired antibodies may be made by isolating antibody-secreting hybridomas elicited using CTLA-4 or a peptide fragment thereof. A representative human CTLA-4 polypeptide (NCBI Sequence NP_005205.2; including a 35 amino acid residue signal sequence (shown underlined) and the 188 amino acid residues of the mature protein) has the amino acid sequence (SEQ ID NO:75):

MACLGFQRHK AQLNLATRTW PCTLLFFLLF IPVFCKAMHV
AQPAVVLASS RGIASFVCEY ASPGKATEVR VTVLRQADSQ
VTEVCAATYM MGNELTFLDD SICTGTSSGN QVNLTIQGLR
AMDTGLYICK VELMYPPPYY LGIGNGTQIY VIDPEPCPDS
DFLLWILAAV SSGLFFYSFL LTAVSLSKML KKRSPLTTGV
YVKMPPTEPE CEKQFQPYFI PIN

Preferred anti-CTLA-4 binding molecules (e.g., antibodies) useful in the generation of the PD-1×CTLA-4 bispecific molecules of the instant invention possess the VL and/or VH Domains of the anti-human CTLA-4 monoclonal antibody “CTLA-4 mAb 1” (ipilimumab, CAS Reg. No.: 477202-00-9, also known as MDX010, and marketed as YERVOY® by Bristol-Myers Squibb); “CTLA-4 mAb 2” (tremelimumab, CAS Reg. No.: 745013-59-6, also known as CP-675206); “CTLA-4 mAb 3” (4B6 as provided in Table 7) or any of the other anti-CTLA-4 antibodies in Table 7; and more preferably possess 1, 2 or all 3 of the CDR_{L}S of the VL Region and/or 1, 2 or all 3 of the CDR_{H}S of the VH Domain of such anti-CTLA-4 monoclonal antibodies.

A. CTLA-4 mAb 1

The amino acid sequence of the VH Domain of CTLA-4 mAb 1 (SEQ ID NO:76) is shown below (CDR_{H} residues are shown underlined).

QVQLVESGGG VVQPGRSLRL SCAASGFTFS SYTMHWVRQA
PGKGLEWVTF ISYDGNNKYY ADSVKGRFTI SRDNSKNTLY
LQMNSLRAED TAIYYCARTG WLGPFDYWGQ GTLVTVSS

The amino acid sequence of the VL Domain of CTLA-4 mAb 1 (SEQ ID NO:77) is shown below (CDR_{L} residues are shown underlined).

EIVLTQSPGT LSLSPGERAT LSCRASQSVG SSYLAWYQQK
PGQAPRLLIY GAFSRATGIP DRFSGSGSGT DFTLTISRLE
PEDFAVYYCQ QYGSSPWTFG QGTKVEIK

B. CTLA-4 mAb 2

The amino acid sequence of the VH Domain of CTLA-4 mAb 2 (SEQ ID NO:78) is shown below (CDR_{H} residues are shown underlined).

QVQLVESGGG VVQPGRSLRL SCAASGFTFS SYGMHWVRQA
PGKGLEWVAV IWYDGSNKYY ADSVKGRFTI SRDNSKNTLY
LQMNSLRAED TAVYYCARDP RGATLYYYYY GMDVWGQGTT
VTVSS

The amino acid sequence of the VL Domain of CTLA-4 mAb 2 (SEQ ID NO:79) is shown below (CDR_{L} residues are shown underlined).

DIQMTQSPSS LSASVGDRVT ITCRASQSIN SYLDWYQQKP
GKAPKLLIYA ASSLQSGVPS RFSGSGSGTD FTLTISSLQP
EDFATYYCQQ YYSTPFTFGP GTKVEIK

C. CTLA-4 mAb 3

The amino acid sequence of the VH Domain of CTLA-4 mAb 3 (SEQ ID NO:90) is shown below (CDR_{H} residues are shown underlined).

QVQLVESGGG VVQPGRSLRL SCAASGFTFS SYTMHWVRQA
PGKGLEWVTF ISYDGSNKHY ADSVKGRFTV SRDNSKNTLY
LQMNSLRAED TAIYYCARTG WLGPFDYWGQ GTLVTVSS

The amino acid sequence of the VL Domain of CTLA-4 mAb 3 (SEQ ID NO:91) is shown below (CDR_{L} residues are shown underlined).

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIK

D. Additional Anti-CTLA-4 Antibodies

Additional anti-CTLA-4 antibodies which may be utilized to generate the PD-1×CTLA-4 bispecific molecules of the instant invention are provided in Table 7.

TABLE 7
Additional Anti-CTLA-4 Antibodies

| CTLA-4 Antibodies | Reference/Source |
|---|---|
| mAb 26 | U.S. Pat. No. 7,034,121; PCT Patent |
| | Publication WO 01/54732 |
| 10D1; 1E2; and 4B6 | U.S. Pat. Nos. 6,984,720; 7,605,238; |
| | 8,017,114; 8,318,916; and 8,784,815; |
| | PCT Patent Publication WO 01/14424 |
| 2.1.3; 3.1.1; 4.1.1; 4.8.1; | U.S. Pat. Nos. 6,682,736; 7,109,003; |
| 4.9.1; 4.10.2; 4.13.1; 4.14.3; | 7,132,281; 7,411,057; 7,807,797; |
| 6.1.1; 11.2.1; 11.6.1; 11.7.1; | 7,824,679; 8,143,379; 8,491,895; and |
| 12.2.1; 12.3.1; 12.3.1.1; | 8,883,984; PCT |
| 12.9.1; and 12.9.1.1 | Patent Publication WO 00/37504 |
| 3B10; 8H5; 8H5-1B1; 3B10- | U.S. Patent Publication 2014/0105914; |
| 4F7; 7B9-1A3; 2C7-1G10; | PCT Patent Publication WO 2012/120125 |
| 3B10-6E3; and 8H5-1A1 | |
| 3.7F10A2; 4.3F6B5; | U.S. Patent Publication 2009/0123477; |
| 4.4A7F4; 4.6C1E3; | PCT Patent Publication WO 2006/066568 |
| 4.7A8H8; 4.7E11F1; | |
| 4.8H10H5; TGN2122; and | |
| TGN2422 | |
| L3D10; L1B11; K4G4; | U.S. Patent Publication 2009/0252741; |
| KM10; and YL2 | PCTPatent Publication WO 2006/029219 |

E. Exemplary anti-CTLA-4 Antibodies

An exemplary anti-CTLA-4 antibody designated “CTLA-4 mAb 3 G1AA” comprises a heavy chain having the VH Domain of CTLA-4 mAb 3 (SEQ ID NO:90), an IgG1 CH1 Domain (SEQ ID NO:40), an IgG1 Hinge (SEQ ID NO:33), and IgG1 CH2-CH3 Domains the substitutions L234A/L235A (SEQ ID NO:43).

The amino acid sequence of the complete heavy chain of CTLA-4 mAb 3 G1AA (SEQ ID NO:92) is shown below.

QVQLVESGGG VVQPGRSLRL SCAASGFTFS SYTMHWVRQA
PGKGLEWVTF ISYDGSNKHY ADSVKGRFTV SRDNSKNTLY
LQMNSLRAED TAIYYCARTG WLGPFDYWGQ GTLVTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLTCLV KGFYPSDIAV EWESNGQPEN NYKTTPPVLD
SDGSFFLYSK LTVDKSRWQQ GNVFSCSVMH EALHNHYTQK
SLSLSPGK

An alternative exemplary anti-CTLA-4 antibody designated “CTLA-4 mAb 3 G4P” comprises a heavy chain having the VH Domain of CTLA-4 mAb 3 (SEQ ID NO:90), an IgG4 CH1 Domain (SEQ ID NO:42), a stabilized IgG4 Hinge (SEQ ID NO:36), and IgG4 CH2-CH3 Domains lacking the C-terminal lysine (SEQ ID NO:4). The amino acid sequence of the complete heavy chain of CTLA-4 mAb 3 G4P is shown below (SEQ ID NO:93).

QVQLVESGGG VVQPGRSLRL SCAASGFTFS SYTMHWVRQA
PGKGLEWVTF ISYDGSNKHY ADSVKGRFTV SRDNSKNTLY
LQMNSLRAED TAIYYCARTG WLGPFDYWGQ GTLVTVSSAS
TKGPSVFPLA PCSRSTSEST AALGCLVKDY FPEPVTVSWN
SGALTSGVHT FPAVLQSSGL YSLSSVVTVP SSSLGTKTYT
CNVDHKPSNT KVDKRVESKY GPPCPPCPAP EFLGGPSVFL
FPPKPKDTLM ISRTPEVTCV VVDVSQEDPE VQFNWYVDGV
EVHNAKTKPR EEQFNSTYRV VSVLTVLHQD WLNGKEYKCK
VSNKGLPSSI EKTISKAKGQ PREPQVYTLP PSQEEMTKNQ
VSLTCLVKGF YPSDIAVEWE SNGQPENNYK TTPPVLDSDG
SFFLYSRLTV DKSRWQEGNV FSCSVMHEAL HNHYTQKSLS
LSLG

The amino acid sequence of the complete light chain of CTLA-4 mAb 3 G1AA and CTLA-4 mAb 3 G4P (SEQ ID NO:94) is shown below.

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC

The exemplary anti-CTLA-4 antibodies, CTLA-4 mAb 3 G1AA and CTLA-4 mAb 3 G4P, both comprise a light chain having the VL Domain of CTLA-4 mAb 3 (SEQ ID NO:91) and a kappa CL (SEQ ID NO:38).

### VIII. Exemplary PD-1×CTLA-4 Bispecific Molecules

A. Exemplary Four Chain Fc Region-Containing Diabodies Having E/K-Coils

Three exemplary PD-1×CTLA-4 bispecific, four-chain, Fc Region-containing diabodies, comprising E/K-coil Heterodimer-Promoting Domains were generated (designated “DART B,” “DART C,” and “DART D”). The structure of these Fc Region-containing diabodies is detailed below. These exemplary PD-1×CTLA-4 diabodies are intended to illustrate, but in no way limit, the scope of the invention.

1. DART B

DART B is a bispecific, four-chain, Fc Region-containing diabody having two binding sites specific for PD-1, two binding sites specific for CTLA-4, a variant IgG4 Fc Region engineered for extended half-life, and E/K-coil Heterodimer-Promoting Domains. The first and third polypeptide chains of DART B comprise, in the N-terminal to C-terminal direction: an N-terminus, a VL Domain of a monoclonal antibody capable of binding to CTLA-4 (VL_{CTLA-4} CTLA-4 mAb 1 VL) (SEQ ID NO:77); an intervening linker peptide (Linker 1: GGGSGGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (E-coil) Domain (EVAACEK-EVAALEK-EVAALEK-EVAALEK (SEQ ID NO:20)); a stabilized IgG4 hinge region (SEQ ID NO:36); a variant of an IgG4 CH2-CH3 Domain comprising substitutions M252Y/S254T/T256E and lacking the C-terminal residue (SEQ ID NO:81); and a C-terminus.

The amino acid sequence of the first and third polypeptide chains of DART B is (SEQ ID NO:95):

EIVLTQSPGT LSLSPGERAT LSCRASQSVG SSYLAWYQQK
PGQAPRLLIY GAFSRATGIP DRFSGSGSGT DFTLTISRLE
PEDFAVYYCQ QYGSSPWTFG QGTKVEIKGG GSGGGGQVQL
VQSGAEVKKP GASVKVSCKA SGYSFTSYWM NWVRQAPGQG
LEWIGVIHPS DSETWLDQKF KDRVTITVDK STSTAYMELS
SLRSEDTAVY YCAREHYGTS PFAYWGQGTL VTVSSGGCGG
GEVAACEKEV AALEKEVAAL EKEVAALEKE SKYGPPCPPC
PAPEFLGGPS VFLFPPKPKD TLYITREPEV TCVVVDVSQE
DPEVQFNWYV DGVEVHNAKT KPREEQFNST YRVVSVLTVL
HQDWLNGKEY KCKVSNKGLP SSIEKTISKA KGQPREPQVY
TLPPSQEEMT KNQVSLTCLV KGFYPSDIAV EWESNGQPEN
NYKTTPPVLD SDGSFFLYSR LTVDKSRWQE GNVFSCSVMH
EALHNHYTQK SLSLSLG

The second and fourth polypeptide chains of DART B comprise, in the N-terminal to C-terminal direction: an N-terminus, a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding CTLA-4 (VH_{CTLA-4} CTLA-4 mAb 1 VH) (SEQ ID NO:76); a cysteine-containing intervening linker peptide (Linker 2: GGCGGG (SEQ ID NO:6)); a cysteine-containing Heterodimer-Promoting (K-coil) Domain (KVAACKE-KVAALKE-KVAALKE-KVAALKE (SEQ ID NO:21); and a C-terminus.

The amino acid sequence of the second and fourth polypeptide chains of DART B is (SEQ ID NO:96):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVESGGGV VQPGRSLRLS CAASGFTFSS YTMHWVRQAP
GKGLEWVTFI SYDGNNKYYA DSVKGRFTIS RDNSKNTLYL
QMNSLRAEDT AIYYCARTGW LGPFDYWGQG TLVTVSSGGC
GGGKVAACKE KVAALKEKVA ALKEKVAALK E

2. DART C

DART C is a bispecific, four-chain, Fe Region-containing diabody having two binding sites specific for PD-1, two binding sites specific for CTLA-4, a variant IgG4 Fc Region engineered for extended half-life, and E/K-coil Heterodimer-Promoting Domains. The first and third polypeptide chains of DART C comprise, in the N-terminal to C-terminal direction: an N-terminus, a VL Domain of a monoclonal antibody capable of binding to CTLA-4 (VL_{CTLA-4} CTLA-4 mAb 3 VL) (SEQ ID NO:91); an intervening linker peptide (Linker 1: GGGSGGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (E-coil) Domain (EVAACEK-EVAALEK-EVAALEK-EVAALEK (SEQ ID NO:20)); a stabilized IgG4 hinge region (SEQ ID NO:36); a variant of an IgG4 CH2-CH3 Domain comprising substitutions M252Y/S254T/T256E and lacking the C-terminal residue (SEQ ID NO:81); and a C-terminus.

The amino acid sequence of the first and third polypeptide chains of DART C is (SEQ ID NO:97):

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIKGG GSGGGGQVQL
VQSGAEVKKP GASVKVSCKA SGYSFTSYWM NWVRQAPGQG
LEWIGVIHPS DSETWLDQKF KDRVTITVDK STSTAYMELS
SLRSEDTAVY YCAREHYGTS PFAYWGQGTL VTVSSGGCGG
GEVAACEKEV AALEKEVAAL EKEVAALEKE SKYGPPCPPC
PAPEFLGGPS VFLFPPKPKD TLYITREPEV TCVVVDVSQE
DPEVQFNWYV DGVEVHNAKT KPREEQFNST YRVVSVLTVL
HQDWLNGKEY KCKVSNKGLP SSIEKTISKA KGQPREPQVY
TLPPSQEEMT KNQVSLTCLV KGFYPSDIAV EWESNGQPEN
NYKTTPPVLD SDGSFFLYSR LTVDKSRWQE GNVFSCSVMH
EALHNHYTQK SLSLSLG

The second and fourth polypeptide chains of DART C comprise, in the N-terminal to C-terminal direction: an N-terminus, a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding CTLA-4 (VH_{CTLA-4} CTLA-4 mAb 3 VH) (SEQ ID NO:90); a cysteine-containing intervening linker peptide (Linker 2: GGCGGG (SEQ ID NO:6)); a cysteine-containing Heterodimer-Promoting (K-coil) Domain (KVAACKE-KVAALKE-KVAALKE-KVAALKE (SEQ ID NO:21); and a C-terminus.

The amino acid sequence of the second and fourth polypeptide chains of DART C is (SEQ ID NO:98):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVESGGGV VQPGRSLRLS CAASGFTFSS YTMHWVRQAP
GKGLEWVTFI SYDGSNKHYA DSVKGRFTVS RDNSKNTLYL
QMNSLRAEDT AIYYCARTGW LGPFDYWGQG TLVTVSSGGC
GGGKVAACKE KVAALKEKVA ALKEKVAALK E

3. DART D

DART D is a bispecific, four-chain, Fc Region-containing diabody having two binding sites specific for PD-1, two binding sites specific for CTLA-4, a variant IgG4 Fc Region engineered for extended half-life, and E/K-coil Heterodimer-Promoting Domains. The first and third polypeptide chains of DART D comprise, in the N-terminal to C-terminal direction: an N-terminus, a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding CTLA-4 (VH_{CTLA-4} CTLA-4 mAb 3 VH) (SEQ ID NO:90); a cysteine-containing intervening linker peptide (Linker 2: GGCGGG (SEQ ID NO:6)); a cysteine-containing Heterodimer-Promoting (E-coil) Domain (EVAACEK-EVAALEK-EVAALEK-EVAALEK (SEQ ID NO:20)); a stabilized IgG4 hinge region (SEQ ID NO:36); a variant of an IgG4 CH2-CH3 Domain comprising substitutions M252Y/S254T/T256E and lacking the C-terminal residue (SEQ ID NO:81); and a C-terminus.

The amino acid sequence of the first and third polypeptide chains of DART D is (SEQ ID NO:99):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVESGGGV VQPGRSLRLS CAASGFTFSS YTMHWVRQAP
GKGLEWVTFI SYDGSNKHYA DSVKGRFTVS RDNSKNTLYL
QMNSLRAEDT AIYYCARTGW LGPFDYWGQG TLVTVSSGGC
GGGEVAACEK EVAALEKEVA ALEKEVAALE KESKYGPPCP
PCPAPEFLGG PSVFLFPPKP KDTLYITREP EVTCVVVDVS
QEDPEVQFNW YVDGVEVHNA KTKPREEQFN STYRVVSVLT
VLHQDWLNGK EYKCKVSNKG LPSSIEKTIS KAKGQPREPQ
VYTLPPSQEE MTKNQVSLTC LVKGFYPSDI AVEWESNGQP
ENNYKTTPPV LDSDGSFFLY SRLTVDKSRW QEGNVFSCSV
MHEALHNHYT QKSLSLSLG

The second and fourth polypeptide chains of DART D comprise, in the N-terminal to C-terminal direction: an N-terminus, a VL Domain of a monoclonal antibody capable of binding to CTLA-4 (VL_{CTLA-4} CTLA-4 mAb 3 VL) (SEQ ID NO:91); an intervening linker peptide (Linker 1: GGGSGGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (K-coil) Domain (KVAACKE-KVAALKE-KVAALKE-KVAALKE (SEQ ID NO:21); and a C-terminus.

The amino acid sequence of the second and fourth polypeptide chains of DART D is (SEQ ID NO:100):

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIKGG GSGGGGQVQL
VQSGAEVKKP GASVKVSCKA SGYSFTSYWM NWVRQAPGQG
LEWIGVIHPS DSETWLDQKF KDRVTITVDK STSTAYMELS
SLRSEDTAVY YCAREHYGTS PFAYWGQGTL VTVSSGGCGG
GKVAACKEKV AALKEKVAAL KEKVAALKE

4. DART F

DART F is a bispecific, four-chain, Fc Region-containing diabody having two binding sites specific for PD-1, two binding sites specific for CTLA-4, a variant IgG1 Fc Region engineered to reduce/eliminate effector function and to extend half-life, and E/K-coil Heterodimer-Promoting Domains. The first and third polypeptide chains of DART F comprise, in the N-terminal to C-terminal direction: an N-terminus, a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding CTLA-4 (VH_{CTLA-4} CTLA-4 mAb 3 VH) (SEQ ID NO:90); a cysteine-containing intervening linker peptide (Linker 2: GGCGGG (SEQ ID NO:6)); a cysteine-containing Heterodimer-Promoting (E-coil) Domain (EVAACEK-EVAALEK-EVAALEK-EVAALEK (SEQ ID NO:20)); a linker (SEQ ID NO:30); a variant of an IgG1 CH2-CH3 Domain comprising substitutions L235A/L235A/M252Y/S254T/T256E and lacking the C-terminal residue (SEQ ID NO:80); and a C-terminus.

The amino acid sequence of the first and third polypeptide chains of DART F (SEQ ID NO:101) is:

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVESGGGV VQPGRSLRLS CAASGFTFSS YTMHWVRQAP
GKGLEWVTFI SYDGSNKHYA DSVKGRFTVS RDNSKNTLYL
QMNSLRAEDT AIYYCARTGW LGPFDYWGQG TLVTVSSGGC
GGGEVAACEK EVAALEKEVA ALEKEVAALE KLEPKSADKT
HTCPPCPAPE AAGGPSVFLF PPKPKDTLYI TREPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLTCLVKGFY PSDIAVEWES
NGQPENNYKT TPPVLDSDGS FFLYSKLTVD KSRWQQGNVF
SCSVMHEALH NHYTQKSLSL SPG

The second and fourth polypeptide chains of DART F comprise, in the N-terminal to C-terminal direction: an N-terminus, a VL Domain of a monoclonal antibody capable of binding to CTLA-4 (VL_{CTLA-4} CTLA-4 mAb 3 VL) (SEQ ID NO:91); an intervening linker peptide (Linker 1: GGGSGGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (K-coil) Domain (KVAACKE-KVAALKE-KVAALKE-KVAALKE (SEQ ID NO:21); and a C-terminus.

The amino acid sequence of the second and fourth polypeptide chains of DART F is the same as that of the second and fourth polypeptide chains of DART D (SEQ ID NO:100).

B. Exemplary Four-Chain Fc Region-Containing Diabodies Having CL/CH1 Domains: DART E

An exemplary PD-1×CTLA-4 bispecific, four-chain, Fc Region-containing diabody comprising CL/CH1 Domains designated “DART E” was generated. The structure of this Fc Region-containing diabodies is detailed below. This exemplary PD-1×CTLA-4 diabody is intended to illustrate, but in no way limit, the scope of the invention.

DART E is a bispecific, four-chain, Fc Region-containing diabody having two binding sites specific for PD-1, two binding sites specific for CTLA-4, CL/CH1 Domains, and a variant IgG4 Fc Region engineered for extended half-life. The first and third polypeptide chains of DART E comprise, in the N-terminal to C-terminal direction: an N-terminus; a VL Domain of a monoclonal antibody capable of binding to CTLA-4 (VL_{CTLA-4} CTLA-4 mAb 3 VL) (SEQ ID NO:91); an intervening linker peptide (Linker 1: GGGS GGGG (SEQ ID NO:5)); a VH Domain of a monoclonal antibody capable of binding to PD-1 (VH_{PD-1} PD-1 mAb 6-I VH) (SEQ ID NO:86); an intervening linker peptide (Linker 2: LGGGSG (SEQ ID NO:8)); an IgG4 CH1 Domain (SEQ ID NO:42); a stabilized IgG4 hinge region (SEQ ID NO: 36); a variant of an IgG4 CH2-CH3 Domain comprising substitutions M252Y/S254T/T256E and lacking the C-terminal residue (SEQ ID NO:81); and a C-terminus.

The amino acid sequence of the first and third polypeptide chains of DART E is (SEQ ID NO:102):

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIKGG GSGGGGQVQL
VQSGAEVKKP GASVKVSCKA SGYSFTSYWM NWVRQAPGQG
LEWIGVIHPS DSETWLDQKF KDRVTITVDK STSTAYMELS
SLRSEDTAVY YCAREHYGTS PFAYWGQGTL VTVSSLGGGS
GASTKGPSVF PLAPCSRSTS ESTAALGCLV KDYFPEPVTV
SWNSGALTSG VHTFPAVLQS SGLYSLSSVV TVPSSSLGTK
TYTCNVDHKP SNTKVDKRVE SKYGPPCPPC PAPEFLGGPS
VFLFPPKPKD TLYITREPEV TCVVVDVSQE DPEVQFNWYV
DGVEVHNAKT KPREEQFNST YRVVSVLTVL HQDWLNGKEY
KCKVSNKGLP SSIEKTISKA KGQPREPQVY TLPPSQEEMT
KNQVSLTCLV KGFYPSDIAV EWESNGQPEN NYKTTPPVLD
SDGSFFLYSR LTVDKSRWQE GNVFSCSVMH EALHNHYTQK
SLSLSLG

The second and fourth polypeptide chains of DART E comprise, in the N-terminal to C-terminal direction: an N-terminus; a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding CTLA-4 (VH_{CTLA-4} CTLA-4 mAb 3 VH) (SEQ ID NO:90); an intervening linker peptide (Linker 2: LGGGSG (SEQ ID NO:8)); a Kappa CL Domain (SEQ ID NO:38); and a C-terminus.

The amino acid sequence of the second and fourth polypeptide chains of DART E is (SEQ ID NO:103):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVESGGGV VQPGRSLRLS CAASGFTFSS YTMHWVRQAP
GKGLEWVTFI SYDGSNKHYA DSVKGRFTVS RDNSKNTLYL
QMNSLRAEDT AIYYCARTGW LGPFDYWGQG TLVTVSSLGG
GSGRTVAAPS VFIFPPSDEQ LKSGTASVVC LLNNFYPREA
KVQWKVDNAL QSGNSQESVT EQDSKDSTYS LSSTLTLSKA
DYEKHKVYAC EVTHQGLSSP VTKSFNRGEC

C. Exemplary Trivalent Binding Molecules Containing Fc Regions

Two exemplary PD-1×CTLA-4 bispecific, four-chain, Fe Region-containing trivalent binding molecules were generated (designated “TRIDENT A” and “TRIDENT B”). The structure of these Fc Region-containing trivalent binding molecules is detailed below. Also presented below is a three chain variant designated “TRIDENT C,” which may be generated. These exemplary PD-1×CTLA-4 trivalent binding molecules are intended to illustrate, but in no way limit, the scope of the invention.

1. TRIDENT A

TRIDENT A is a bispecific, four chain, Fc Region-containing trivalent binding molecule having two binding sites specific for PD-1, one binding sites specific for CTLA-4, a variant knob/hole-bearing IgG4 Fc Region engineered for extended half-life, E/K-coil Heterodimer-Promoting Domains and CL/CH1 Domains. The first polypeptide chain of TRIDENT A comprises, in the N-terminal to C-terminal direction: a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (E-coil) Domain (EVAACEK-EVAALEK-EVAALEK-EVAALEK (SEQ ID NO:20)); a stabilized IgG4 hinge region (SEQ ID NO: 36); a knob-bearing IgG4 CH2-CH3 Domain comprising substitutions M252Y/S254T/T256E and lacking the C-terminal residue (SEQ ID NO:84); and a C-terminus.

The amino acid sequence of the first polypeptide chain of TRIDENT A is (SEQ ID NO:104):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVQSGAEV KKPGASVKVS CKASGYSFTS YWMNWVRQAP
GQGLEWIGVI HPSDSETWLD QKFKDRVTIT VDKSTSTAYM
ELSSLRSEDT AVYYCAREHY GTSPFAYWGQ GTLVTVSSGG
CGGGEVAACE KEVAALEKEV AALEKEVAAL EKESKYGPPC
PPCPAPEFLG GPSVFLFPPK PKDTLYITRE PEVTCVVVDV
SQEDPEVQFN WYVDGVEVHN AKTKPREEQF NSTYRVVSVL
TVLHQDWLNG KEYKCKVSNK GLPSSIEKTI SKAKGQPREP
QVYTLPPSQE EMTKNQVSLW CLVKGFYPSD IAVEWESNGQ
PENNYKTTPP VLDSDGSFFL YSRLTVDKSR WQEGNVFSCS
VMHEALHNHY TQKSLSLSLG

The second polypeptide chain of TRIDENT A comprises, in the N-terminal to C-terminal direction: an N-terminus, a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (K-coil) Domain (KVAACKE-KVAALKE-KVAALKE-KVAALKE (SEQ ID NO:21)); and a C-terminus.

The amino acid sequence of the second polypeptide chain of TRIDENT A is (SEQ ID NO:105):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVQSGAEV KKPGASVKVS CKASGYSFTS YWMNWVRQAP
GQGLEWIGVI HPSDSETWLD QKFKDRVTIT VDKSTSTAYM
ELSSLRSEDT AVYYCAREHY GTSPFAYWGQ GTLVTVSSGG
CGGGKVAACK EKVAALKEKV AALKEKVAAL KE

The third polypeptide chains of TRIDENT A comprises, in the N-terminal to C-terminal direction: an N-terminus; a VH Domain of a monoclonal antibody capable of binding CTLA-4 (VH_{CTLA-4} CTLA-4 mAb 3 VH) (SEQ ID NO:90); an IgG4 CH1 Domain (SEQ ID NO:42); a stabilized IgG4 hinge region (SEQ ID NO: 36); a hole-bearing IgG4 CH2-CH3 Domain comprising substitutions M252Y/S254T/T256E and lacking the C-terminal residue (SEQ ID NO:81); and a C-terminus.

The amino acid sequence of the third polypeptide chain of TRIDENT A (SEQ ID NO:106):

QVQLVESGGG VVQPGRSLRL SCAASGFTFS SYTMHWVRQA
PGKGLEWVTF ISYDGSNKHY ADSVKGRFTV SRDNSKNTLY
LQMNSLRAED TAIYYCARTG WLGPFDYWGQ GTLVTVSSAS
TKGPSVFPLA PCSRSTSEST AALGCLVKDY FPEPVTVSWN
SGALTSGVHT FPAVLQSSGL YSLSSVVTVP SSSLGTKTYT
CNVDHKPSNT KVDKRVESKY GPPCPPCPAP EFLGGPSVFL
FPPKPKDTLY ITREPEVTCV VVDVSQEDPE VQFNWYVDGV
EVHNAKTKPR EEQFNSTYRV VSVLTVLHQD WLNGKEYKCK
VSNKGLPSSI EKTISKAKGQ PREPQVYTLP PSQEEMTKNQ
VSLSCAVKGF YPSDIAVEWE SNGQPENNYK TTPPVLDSDG
SFFLVSRLTV DKSRWQEGNV FSCSVMHEAL HNRYTQKSLS
LSLG

The fourth polypeptide chain of TRIDENT A comprises, in the N-terminal to C-terminal direction: an N-terminus; a VL Domain of a monoclonal antibody capable of binding to CTLA-4 (VL_{CTLA-4} CTLA-4 mAb 3 VL) (SEQ ID NO:91); a Kappa CL Domain (SEQ ID NO:38); and a C-terminus.

The amino acid sequence of the fourth polypeptide chain of TRIDENT A is (SEQ ID NO:107):

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC

2. TRIDENT B

TRIDENT B is a bispecific, four-chain, Fc Region-containing trivalent binding molecule having two binding sites specific for PD-1, one binding sites specific for CTLA-4, a variant knob/hole-bearing IgG1 Fc Region engineered to reduce/eliminate effector function and to extend half-life, E/K-coil Heterodimer-Promoting Domains and CL/CH1 Domains. The first polypeptide chain of TRIDENT B comprises, in the N-terminal to C-terminal direction: a VL Domain of a monoclonal antibody capable of binding to PD-1 (VL_{PD-1} PD-1 mAb 6-SQ VL) (SEQ ID NO:87); an intervening linker peptide (Linker 1: GGGS GGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (E-coil) Domain (EVAACEK-EVAALEK-EVAALEK-EVAALEK (SEQ ID NO:20)); a linker (SEQ ID NO: 31); a knob-bearing IgG1 CH2-CH3 Domain comprising substitutions L234A/L235A/M252Y/S254T/T256E and lacking the C-terminal residue (SEQ ID NO:82); and a C-terminus.

The amino acid sequence of the first polypeptide chain of TRIDENT B is (SEQ ID NO:108):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVQSGAEV KKPGASVKVS CKASGYSFTS YWMNWVRQAP
GQGLEWIGVI HPSDSETWLD QKFKDRVTIT VDKSTSTAYM
ELSSLRSEDT AVYYCAREHY GTSPFAYWGQ GTLVTVSSGG
CGGGEVAACE KEVAALEKEV AALEKEVAAL EKGGGDKTHT
CPPCPAPEAA GGPSVFLFPP KPKDTLYITR EPEVTCVVVD
VSHEDPEVKF NWYVDGVEVH NAKTKPREEQ YNSTYRVVSV
LTVLHQDWLN GKEYKCKVSN KALPAPIEKT ISKAKGQPRE
PQVYTLPPSR EEMTKNQVSL WCLVKGFYPS DIAVEWESNG
QPENNYKTTP PVLDSDGSFF LYSKLTVDKS RWQQGNVFSC
SVMHEALHNH YTQKSLSLSP GK

The second polypeptide chain of TRIDENT B comprises, in the N-terminal to C-terminal direction: an N-terminus, a VL Domain of a monoclonal antibody capable of binding to PD-1 (VL_{PD-1} PD-1 mAb 6-SQ VL) (SEQ ID NO:87); an intervening linker peptide (Linker 1: GGGSGGGG (SEQ ID NO:5)); a VH Domain of a monoclonal antibody capable of binding to PD-1 (VH_{PD-1} PD-1 mAb 6-I VH) (SEQ ID NO:86); a cysteine-containing intervening linker peptide (Linker 2: GGCGGG (SEQ ID NO:6)); a cysteine-containing Heterodimer-Promoting (K-coil) Domain (KVAACKE-KVAALKE-KVAALKE-KVAALKE (SEQ ID NO:21)); and a C-terminus.

The amino acid sequence of the second polypeptide chain of TRIDENT B is the same as that of the second polypeptide chain of TRIDENT A (SEQ ID NO:105):

The third polypeptide chains of TRIDENT B comprises, in the N-terminal to C-terminal direction: an N-terminus; a VH Domain of a monoclonal antibody capable of binding CTLA-4 (VH_{CTLA-4} CTLA-4 mAb 3 VH) (SEQ ID NO:90); an IgG1 CH1 Domain (SEQ ID NO:40); an IgG1 hinge region (SEQ ID NO:33); a hole-bearing IgG1 CH2-CH3 Domain comprising substitutions L234A/L235A/M252Y/S254T/T256E and lacking the C-terminal residue (SEQ ID NO:83); and a C-terminus.

The amino acid sequence of the third polypeptide chain of TRIDENT B is (SEQ ID NO:109):

QVQLVESGGG VVQPGRSLRL SCAASGFTFS SYTMHWVRQA
PGKGLEWVTF ISYDGSNKHY ADSVKGRFTV SRDNSKNTLY
LQMNSLRAED TAIYYCARTG WLGPFDYWGQ GTLVTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLYITREPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

The fourth polypeptide chain of TRIDENT B comprises, in the N-terminal to C-terminal direction: an N-terminus; a VL Domain of a monoclonal antibody capable of binding to CTLA-4 (VL_{CTLA-4} CTLA-4 mAb 3 VL) (SEQ ID NO:91); a Kappa CL Domain (SEQ ID NO:38); and a C-terminus.

The amino acid sequence of the fourth polypeptide chain of TRIDENT B is the same as that of the second polypeptide chain of TRIDENT A (SEQ ID NO:107).

3. TRIDENT C

As provided herein, trivalent binding molecules comprising three polypeptide chain may be generated by combining (e.g., fusing encoding polynucleotides, etc.) the binding domains of two separate polypeptide chains into one chain. One bispecific, three-chain, Fc Region-containing trivalent binding molecule that may be generated has two binding sites specific for PD-1, one binding sites specific for CTLA-4, a variant knob/hole-bearing IgG4 Fc Region engineered for extended half-life, and E/K-coil Heterodimer-Promoting Domains (“TRIDENT C”). The first and second polypeptide chains of TRIDENT C may be identical to those of TRIDENT A provided above.

Where the first and second chains are identical to those of TRIDENT A, the third polypeptide chain of TRIDENT C may comprise, in the N-terminal to C-terminal direction: an N-terminus; a VL Domain of a monoclonal antibody capable of binding to CTLA-4 (VL_{CTLA-4} CTLA-4 mAb 3 VL) (SEQ ID NO:91); an intervening spacer peptide (GGGGSGGGGSGGGGS (SEQ ID NO:37)); a VH Domain of a monoclonal antibody capable of binding CTLA-4 (VH_{CTLA-4} CTLA-4 mAb 3 VH) (SEQ ID NO:90); a stabilized IgG4 hinge region (SEQ ID NO: 36); a hole-bearing IgG4 CH2-CH3 Domain comprising substitutions M252Y/S254T/T256E and lacking the C-terminal residue (SEQ ID NO:85); and a C-terminus.

Thus, the amino acid sequence of the third polypeptide chain of TRIDENT C is (SEQ ID NO:110):

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIKGG GGSGGGGSGG
GGSQVQLVES GGGVVQPGRS LRLSCAASGF TFSSYTMHWV
RQAPGKGLEW VTFISYDGSN KHYADSVKGR FTVSRDNSKN
TLYLQMNSLR AEDTAIYYCA RTGWLGPFDY WGQGTLVTVS
SESKYGPPCP PCPAPEFLGG PSVFLFPPKP KDTLYITREP
EVTCVVVDVS QEDPEVQFNW YVDGVEVHNA KTKPREEQFN
STYRVVSVLT VLHQDWLNGK EYKCKVSNKG LPSSIEKTIS
KAKGQPREPQ VYTLPPSQEE MTKNQVSLSC AVKGFYPSDI
AVEWESNGQP ENNYKTTPPV LDSDGSFFLV SRLTVDKSRW
QEGNVFSCSV MHEALHNRYT QKSLSLSLG

### IX. Methods of Production

The PD-1×CTLA-4 bispecific molecules of the present invention are most preferably produced through the recombinant expression of nucleic acid molecules that encode such polypeptides, as is well-known in the art.

Polypeptides of the invention may be conveniently prepared using solid phase peptide synthesis (Merrifield, B. (1986) “Solid Phase Synthesis,” Science 232(4748):341-347; Houghten, R. A. (1985) “General Method For The Rapid Solid-Phase Synthesis Of Large Numbers Of Peptides: Specificity Of Antigen-Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

In an alternative, antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco) or transgenic milk. Suitable methods for expressing antibodies recombinantly in plants or milk have been disclosed (see, for example, Peeters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol Methods 231:147-157). Suitable methods for making derivatives of antibodies, e.g., humanized, single-chain, etc. are known in the art, and have been described above. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

Vectors containing polynucleotides of interest (e.g., polynucleotides encoding the polypeptide chains of the PD-1×CTLA-4 bispecific molecules of the present invention) can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cell capable of overexpressing heterologous DNAs can be used for the purpose of expressing a polypeptide or protein of interest. Non-limiting examples of suitable mammalian host cells include but are not limited to COS, HeLa, and CHO cells.

The invention includes polypeptides comprising an amino acid sequence of the PD-1×CTLA-4 bispecific molecule of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available.

The invention includes variants of PD-1×CTLA-4 bispecific molecules, including functionally equivalent polypeptides that do not significantly affect the properties of such molecules as well as variants that have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues that can be conservatively substituted for one another include but are not limited to: glycine/alanine; serine/threonine; valine/isoleucine/leucine; asparagine/glutamine; aspartic acid/glutamic acid; lysine/arginine; and phenylalanine/tyrosine. These polypeptides also include glycosylated and non-glycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the Variable Domain. Changes in the Variable Domain can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

The invention encompasses fusion proteins comprising one or more of the polypeptides or antibodies of this invention. In one embodiment, a fusion polypeptide is provided that comprises a light chain, a heavy chain or both a light and heavy chain. In another embodiment, the fusion polypeptide contains a heterologous immunoglobulin constant region. In another embodiment, the fusion polypeptide contains a Light Chain Variable Domain and a Heavy Chain Variable Domain of an antibody produced from a publicly-deposited hybridoma. For purposes of this invention, an antibody fusion protein contains one or more polypeptide domains that specifically bind to PD-1 and/or CTLA-4 and another amino acid sequence to which it is not attached in the native molecule, for example, a heterologous sequence or a homologous sequence from another region.

### X. Uses of the PD-1×CTLA-4 Bispecific Molecules of the Present Invention

The present invention encompasses compositions, including pharmaceutical compositions, comprising the PD-1×CTLA-4 bispecific molecules of the present invention (e.g., bispecific antibodies, bispecific diabodies, trivalent binding molecules, etc.), polypeptides derived from such molecules, polynucleotides comprising sequences encoding such molecules or polypeptides, and other agents as described herein.

As discussed above, both PD-1 and CTLA-4 play important roles in negatively regulating immune responses (e.g., immune cell proliferation, function and homeostasis). The PD-1×CTLA-4 bispecific molecules of the present invention have the ability to inhibit PD-1 function, and thus reverse the PD-1-mediated immune system inhibition. In addition, the PD-1×CTLA-4 bispecific molecules of the present invention have the ability to inhibit CTLA-4 function and thus augment the immune system by blocking immune system inhibition mediated by PD-1 and CTLA-4. The PD-1×CTLA-4 bispecific molecules of the present invention also allow for full blockade of both PD-1 and CTLA-4, as well as blockade that is biased toward CTLA-4 when co-expressed with PD-1. Thus, the PD-1×CTLA-4 bispecific molecules of the invention are useful for relieving T-cell exhaustion and/or augmenting an immune response (e.g., a T-cell and/or NK-cell mediated immune response) of a subject. In particular, the PD-1×CTLA-4 bispecific molecules of the invention and may be used to treat any disease or condition associated with an undesirably suppressed immune system, including cancer and diseases that are associated with the presence of a pathogen (e.g., a bacterial, fungal, viral or protozoan infection).

The cancers that may be treated by the PD-1×CTLA-4 bispecific molecules of the present invention include cancers characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumor, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, a hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

In particular, PD-1×CTLA-4 bispecific molecules of the present invention may be used in the treatment of colorectal cancer, hepatocellular carcinoma, glioma, kidney cancer, breast cancer, multiple myeloma, bladder cancer, neuroblastoma; sarcoma, non-Hodgkin's lymphoma, non-small cell lung cancer, ovarian cancer, pancreatic cancer and rectal cancer.

Infections that may be treated by the PD-1×CTLA-4 bispecific molecules of the present invention include chronic viral, bacterial, fungal and parasitic infections. Chronic infections that may be treated by the PD-1×CTLA-4 bispecific molecules of the present invention include Epstein Barr virus, Hepatitis A Virus (HAV); Hepatitis B Virus (HBV); Hepatitis C Virus (HCV); herpes viruses (e.g. HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia lambia, trichinella, Dientamoeba Fragilis, Trypanosoma brucei, Trypanosoma cruzi, and Leishmania donovani.

### XI. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of the PD-1×CTLA-4 bispecific molecules of the present invention, or a combination of such agents and a pharmaceutically acceptable carrier. Preferably, compositions of the invention comprise a prophylactically or therapeutically effective amount of the PD-1×CTLA-4 bispecific molecules of the present invention and a pharmaceutically acceptable carrier. The invention also encompasses such pharmaceutical compositions that additionally include a second therapeutic antibody (e.g., tumor-specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with a PD-1×CTLA-4 bispecific molecule of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the PD-1×CTLA-4 bispecific molecules of the present invention. The kit can further comprise one or more other prophylactic and/or therapeutic agents useful for the treatment of cancer, in one or more containers.

### XII. Methods of Administration

The compositions of the present invention may be provided for the treatment, prophylaxis, and amelioration of one or more symptoms associated with a disease, disorder or infection by administering to a subject an effective amount of a fusion protein or a conjugated molecule of the invention, or a pharmaceutical composition comprising a fusion protein or a conjugated molecule of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262:4429-4432), construction of a nucleic acid as part of a retroviral or other vector, etc.

Methods of administering a molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the PD-1×CTLA-4 bispecific molecules of the present invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that preparations of the PD-1×CTLA-4 bispecific molecules of the present invention are packaged in a hermetically sealed container such as an ampoule or sachette indicating the quantity of the molecule. In one embodiment, such molecules are supplied as a dry sterilized lyophilized powder or water free concentrate in a hermetically sealed container and can be reconstituted, e.g., with water or saline to the appropriate concentration for administration to a subject. Preferably, the PD-1×CTLA-4 bispecific molecules of the present invention are supplied as a dry sterile lyophilized powder in a hermetically sealed container.

The lyophilized preparations of the PD-1×CTLA-4 bispecific molecules of the present invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such PD-1×CTLA-4 bispecific molecules when provided in liquid form are supplied in a hermetically sealed container.

The amount of such preparations of the invention that will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

As used herein, an “effective amount” of a pharmaceutical composition, in one embodiment, is an amount sufficient to effect beneficial or desired results including, without limitation, clinical results such as decreasing symptoms resulting from the disease, attenuating a symptom of infection (e.g., viral load, fever, pain, sepsis, etc.) or a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

An effective amount can be administered in one or more administrations. For purposes of this invention, an effective amount of drug, compound, or pharmaceutical composition is an amount sufficient: to kill and/or reduce the proliferation of cancer cells, and/or to eliminate, reduce and/or delay the development of metastasis from a primary site of cancer; or to reduce the proliferation of (or the effect of) an infectious pathogen and to reduce and/or delay the development of the pathogen-mediated disease, either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art.

For the PD-1×CTLA-4 bispecific molecules encompassed by the invention, the dosage administered to a patient is preferably determined based upon the body weight (kg) of the recipient subject. For the PD-1×CTLA-4 bispecific molecules encompassed by the invention, the dosage administered to a patient is typically from about 0.01 μg/kg to about 150 mg/kg or more of the subject's body weight.

The dosage and frequency of administration of a PD-1×CTLA-4 bispecific molecule of the present invention may be reduced or altered by enhancing uptake and tissue penetration of the molecule by modifications such as, for example, lipidation.

The dosage of a PD-1×CTLA-4 bispecific molecule of the invention administered to a patient may be calculated for use as a single agent therapy. Alternatively, the molecule may be used in combination with other therapeutic compositions and the dosage administered to a patient are lower than when the molecules are used as a single agent therapy.

The pharmaceutical compositions of the invention may be administered locally to the area in need of treatment; this may be achieved by, for example, and not by way of limitation, local infusion, by injection, or by means of an implant, the implant being of a porous, non-porous, or gelatinous material, including membranes, such as sialastic membranes, or fibers. Preferably, when administering a molecule of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 3 17-327).

Where the composition of the invention is a nucleic acid encoding a PD-1×CTLA-4 bispecific molecule of the present invention, the nucleic acid can be administered in vivo to promote expression of its encoded PD-1×CTLA-4 bispecific molecule by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (See e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

Treatment of a subject with a therapeutically or prophylactically effective amount of a PD-1×CTLA-4 bispecific molecule of the present invention can include a single treatment or, preferably, can include a series of treatments. In a preferred example, a subject is treated with such a diabody one time per week for between about 1 to 10 weeks, preferably between 2 to 8 weeks, more preferably between about 3 to 7 weeks, and even more preferably for about 4, 5, or 6 weeks. The pharmaceutical compositions of the invention can be administered once a day, twice a day, or three times a day. Alternatively, the pharmaceutical compositions can be administered once a week, twice a week, once every two weeks, once a month, once every six weeks, once every two months, twice a year or once per year. It will also be appreciated that the effective dosage of the molecules used for treatment may increase or decrease over the course of a particular treatment.

### XIII. Exemplary Embodiments

The invention is particularly directed to the embodiments E1-E26:
E1. A bispecific molecule possessing both one or more epitope-binding sites capable of immunospecific binding to (an) epitope(s) of PD-1 and one or more epitope-binding sites capable of immunospecific binding to (an) epitope(s) of CTLA-4, wherein such molecule comprises:
   (A) a Heavy Chain Variable Domain and a Light Chain Variable Domain of an antibody that binds PD-1; and
   (B) a Heavy Chain Variable Domain and a Light Chain Variable Domain of an antibody that binds CTLA-4;
   wherein such molecule is:
   (i) a diabody, such diabody being a covalently bonded complex that comprises two, three, four or five polypeptide chains; or
   (ii) a trivalent binding molecule, such trivalent binding molecule being a covalently bonded complex that comprises three, four, five, or more polypeptide chains.
E2. The bispecific molecule of Embodiment E1, wherein such molecule exhibits an activity that is enhanced relative to such activity exhibited by two monospecific molecules one of which possesses such Heavy Chain Variable Domain and such Light Chain Variable Domain of such antibody that binds PD-1 and the other of which possesses such Heavy Chain Variable Domain and such Light Chain Variable Domain of such antibody that binds CTLA-4.
E3. The bispecific molecule of Embodiment E1 or E2, wherein such molecule elicits fewer immune-related adverse events (irAEs) when administered to a subject in need thereof relative to such iREs elicited by the administration of a monospecific antibody that binds CTLA-4.
E4. The bispecific molecule of Embodiment E3, wherein said monospecific antibody that binds CTLA-4 is ipilimumab.
E5. The bispecific molecule of any one of Embodiments E1-E4, wherein such molecule comprises an Fc Region.
E6. The bispecific molecule of Embodiment E5, wherein such Fc Region is a variant Fc Region that comprises:
   (A) one or more amino acid modifications that reduces the affinity of the variant Fc Region for an FcγR; and/or
   (B) one or more amino acid modifications that enhances the serum half-life of the variant Fc Region.
E7. The bispecific molecule of Embodiment E6, wherein such modifications that reduces the affinity of the variant Fc Region for an FcγR comprise the substitution of L234A; L235A; or L234A and L235A, wherein such numbering is that of the EU index as in Kabat.
E8. The bispecific molecule of Embodiment E6 or E7, wherein such modifications that that enhances the serum half-life of the variant Fc Region comprise the substitution of M252Y; M252Y and S254T; M252Y and T256E; M252Y, S254T and T256E; or K288D and H435K, wherein such numbering is that of the EU index as in Kabat.
E9. The bispecific molecule of any one of Embodiments E1-E8, wherein such molecule is such diabody and comprises two epitope-binding sites capable of immunospecific binding to an epitope of PD-1 and two epitope-binding sites capable of immunospecific binding to an epitope of CTLA-4.
E10. The bispecific molecule of any one of Embodiments E1-E8, wherein such molecule is such trivalent binding molecule and comprises two epitope-binding sites capable of immunospecific binding to an epitope of PD-1 and one epitope-binding site capable of immunospecific binding to an epitope of CTLA-4.
E11. The bispecific molecule of any one of Embodiments E1-E10, wherein such molecule is capable of binding to PD-1 and CTLA-4 molecules present on the cell surface.
E12. The bispecific molecule of any one of Embodiments E1-E11, wherein such molecule is capable of simultaneously binding to PD-1 and CTLA-4.
E13. The bispecific molecule of any one of Embodiments E1-E12, wherein such molecule promotes the stimulation of immune cells.
E14. The bispecific molecule of Embodiment E13, wherein such stimulation of immune cells results in:
   (A) immune cell proliferation; and/or
   (B) immune cell production and/or release of at least one cytokine; and/or
   (C) immune cell production and/or release of at least one lytic molecule; and/or
   (D) immune cell expression of at least one activation marker.
E15. The bispecific molecule of Embodiment E13 or E14, wherein such immune cell is a T-lymphocyte or an NK-cell.
E16. The bispecific molecule of any one of Embodiments E1-E15, wherein such epitope-binding sites capable of immunospecific binding to an epitope of PD-1 comprise:
   (A) the VH Domain of PD-1 mAb 1 (SEQ ID NO:47) and the VL Domain of PD-1 mAb 1 (SEQ ID NO:48); or
   (B) the VH Domain of PD-1 mAb 2 (SEQ ID NO:49) and the VL Domain of PD-1 mAb 2 (SEQ ID NO:50); or
   (C) the VH Domain of PD-1 mAb 3 (SEQ ID NO:51) and the VL Domain of PD-1 mAb 3 (SEQ ID NO:52); or
   (D) the VH Domain of PD-1 mAb 4 (SEQ ID NO:53) and the VL Domain of PD-1 mAb 4 (SEQ ID NO:54); or
   (E) the VH Domain of PD-1 mAb 5 (SEQ ID NO:55) and the VL Domain of PD-1 mAb 5 (SEQ ID NO:56); or
   (F) the VH Domain of PD-1 mAb 6 (SEQ ID NO:57) and the VL Domain of PD-1 mAb 6 (SEQ ID NO:58); or
   (G) the VH Domain of PD-1 mAb 6-I VH (SEQ ID NO:86) and the VL Domain of PD-1 mAb 6-SQ VL (SEQ ID NO:87); or
   (H) the VH Domain of PD-1 mAb 7 (SEQ ID NO:59) and the VL Domain of PD-1 mAb 7 (SEQ ID NO:60); or
   (I) the VH Domain of PD-1 mAb 8 (SEQ ID NO:61) and the VL Domain of PD-1 mAb 8 (SEQ ID NO:62).
E17. The bispecific molecule of any one of Embodiments E1-E16, wherein such epitope-binding site(s) capable of immunospecific binding to an epitope of CTLA-4 comprise:
   (A) the VH Domain of CTLA-4 mAb 1 (SEQ ID NO:76) and the VL Domain of CTLA-4 mAb 1 (SEQ ID NO:77); or
   (B) the VH Domain of CTLA-4 mAb 2 (SEQ ID NO:78) and the VL Domain of CTLA-4 mAb 2 (SEQ ID NO:79); or
   (C) the VH Domain of CTLA-4 mAb 3 (SEQ ID NO:90) and the VL Domain of CTLA-4 mAb 3 (SEQ ID NO:91).
E18. The bispecific molecule of Embodiment 17, wherein:
   (A) such epitope-binding sites capable of immunospecific binding to an epitope of PD-1 comprise the VH Domain of PD-1 mAb 6-I VH (SEQ ID NO:86) and the VL Domain of PD-1 mAb 6-SQ (SEQ ID NO:87); and
   (B) such epitope-binding site(s) capable of immunospecific binding to an epitope of CTLA-4 comprise(s) the VH Domain of CTLA-4 mAb 3 (SEQ ID NO:90) and the VL Domain of CTLA-4 mAb 3 (SEQ ID NO:91).
E19. The bispecific molecule of any one of Embodiments E1-E18, wherein such molecule comprises:
   (A) two polypeptide chains having SEQ ID NO:95, and two polypeptide chain having SEQ ID NO:96; or
   (B) two polypeptide chains having SEQ ID NO:97, and two polypeptide chain having SEQ ID NO:98; or
   (C) two polypeptide chains having SEQ ID NO:99, and two polypeptide chain having SEQ ID NO:100; or
   (D) two polypeptide chains having SEQ ID NO:102, and two polypeptide chain having SEQ ID NO:103; or
   (E) two polypeptide chains having SEQ ID NO:101, and two polypeptide chain having SEQ ID NO:100; or
   (F) one polypeptide chains having SEQ ID NO:104, one polypeptide chain having SEQ ID NO:105, one polypeptide chain having SEQ ID NO:106, and one polypeptide chain having SEQ ID NO:107; or
   (G) one polypeptide chains having SEQ ID NO:108, one polypeptide chain having SEQ ID NO:105, one polypeptide chain having SEQ ID NO:109, and one polypeptide chain having SEQ ID NO:107.
E20. A pharmaceutical composition that comprises an effective amount of the bispecific molecule of any of Embodiments E1-E19 and a pharmaceutically acceptable carrier.
E21. The bispecific molecule of any one of Embodiments E1-E19, wherein such molecule is used to promote stimulation of an immune-mediated response of a subject in need thereof.
E22. The bispecific molecule of any one of Embodiments E1-E19, wherein such molecule is used in the treatment of a disease or condition associated with a suppressed immune system.
E23. The bispecific molecule of Embodiment E22, wherein the disease or condition is cancer or an infection.
E24. The bispecific molecule of Embodiment E23, wherein such cancer is characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumor, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, a hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.
E25. The bispecific molecule of Embodiment E24, wherein such infection is characterized by the presence of a bacterial, fungal, viral or protozoan pathogen.
E26. The bispecific molecule of Embodiment E25, wherein such infection is characterized by the presence of Epstein Barr virus, Hepatitis A Virus (HAV); Hepatitis B Virus (HBV); Hepatitis C Virus (HCV); herpes viruses (e.g. HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g. Schistosomia), Giardia lambia, trichinella, Dientamoeba Fragilis, Trypanosoma brucei, Trypanosoma cruzi, and Leishmania donovani.

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### Example 1

### Bispecific Molecules Provide Enhanced Stimulation of Immune Responses

A bispecific molecule having specificity for distinct cell surface proteins that modulate two immunomodulatory pathways, PD-1 and LAG-3, was generated and designated “DART A.”

DART A is a bispecific, four chain, Fc Region-containing diabody having two binding sites specific for PD-1, two binding sites specific for LAG-3, a variant IgG4 Fc Region engineered for extended half-life, and cysteine-containing E/K-coil Heterodimer-Promoting Domains. As provided in more detail below, DART A comprises the binding specificities (i.e., the VH and VL Domains) of a humanized anti-PD-1 antibody (hPD-1 mAb 6) and a humanized anti-LAG-3 antibody (hLAG-3 mAb 1). The amino acid sequence of the first and third polypeptide chains of DART A is (SEQ ID NO:63):

DIQMTQSPSS LSASVGDRVT ITCRASQDVS SVVAWYQQKP
GKAPKLLIYS ASYRYTGVPS RFSGSGSGTD FTLTISSLQP
EDFATYYCQQ HYSTPWTFGG GTKLEIKGGG SGGGGQVQLV
QSGAEVKKPG ASVKVSCKAS GYSFTSYWMN WVRQAPGQGL
EWIGVIHPSD SETWLDQKFK DRVTITVDKS TSTAYMELSS
LRSEDTAVYY CAREHYGTSP FAYWGQGTLV TVSSGGCGGG
EVAACEKEVA ALEKEVAALE KEVAALEKES KYGPPCPPCP
APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLG

In SEQ ID NO:63, amino acid residues 1-107 correspond to the amino acid sequence of a VL Domain of a humanized monoclonal antibody capable of binding to LAG-3 (hLAG-3 mAb 1); residues 108-115 correspond to the intervening spacer peptide (Linker 1: GGGSGGGG (SEQ ID NO:5)); residues 116-234 correspond to the VH Domain of a monoclonal antibody capable of binding to PD-1 (hPD-1 mAb 6, SEQ ID NO:57, wherein X₁ is I); residues 235-240 correspond to an intervening spacer peptide (Linker 2: GGCGGG (SEQ ID NO:6)); residues 241-268 correspond to a cysteine-containing Heterodimer-Promoting (E-coil) Domain (EVAACEK-EVAALEK-EVAALEK-EVAALEK (SEQ ID NO:20)); residues 269-280 correspond to a stabilized IgG4 Hinge Region (SEQ ID NO:36); residues to 281-496 correspond to a variant of IgG4 CH2-CH3 Domain comprising substitutions M252Y/S254T/T256E and lacking the C-terminal residue.

The amino acid sequence of the second and fourth polypeptide chains of DART A is (SEQ ID NO:64):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVQSGAEV KKPGASVKVS CKASGYTFTD YNMDWVRQAP
GQGLEWMGDI NPDNGVTIYN QKFEGRVTMT TDTSTSTAYM
ELRSLRSDDT AVYYCAREAD YFYFDYWGQG TTLTVSSGGC
GGGKVAACKE KVAALKEKVA ALKEKVAALK E

In SEQ ID NO:64, amino acid residues 1-111 correspond to the amino acid sequence of a VL Domain of a monoclonal antibody capable of binding to PD-1 (hPD-1 mAb 6, SEQ ID NO:58 wherein X₁ is S and X₂ is Q); residues 112-119 correspond to an intervening spacer peptide (Linker 1: GGGSGGGG (SEQ ID NO:5)); residues 120-237 correspond to a VH Domain of a humanized monoclonal antibody capable of binding LAG-3 (hLAG-3 mAb 1); residues 238-243 correspond to a cysteine-containing spacer linker peptide (Linker 2: GGCGGG (SEQ ID NO:6)); residues 244-271 correspond to a cysteine-containing Heterodimer-Promoting (K-coil) Domain (KVAACKE-KVAALKE-KVAALKE-KVAALKE (SEQ ID NO:21)).

The ability of DART A to stimulate T-cells was examined in a Staphylococcus aureus enterotoxin type B (“SEB”) assay. SEB is a microbial superantigen capable of activating a large proportion of T-cells (5-30%) in SEB-responsive donors. SEB binds to MHC II outside the peptide binding grove and thus is MHC II dependent, but unrestricted and TCR mediated. SEB-stimulation of T-cells results in oligoclonal T-cell proliferation and cytokine production (although donor variability may be observed). Within 48 hours of SEB-stimulation PMBCs upregulate PD-1 and LAG-3 with a further enhancement at day 5, post-secondary culture in 96-well plate with SEB-stimulation. Upregulation of the immune check point proteins PD-1 and LAG-3 following SEB-stimulation of PBMCs limits cytokine release upon SEB restimulation. The ability of DART A to enhance cytokine release through checkpoint inhibition was examined and compared to the activity of the parental anti-PD-1 and anti-LAG-3 antibodies alone and in combination.

Briefly, PBMCs were purified using the Ficoll-Paque Plus (GE Healthcare) density gradient centrifugation method according to manufacturer's instructions from whole blood obtained under informed consent from healthy donors (Biological Specialty Corporation) and T-cells were then purified using the Dynabeads® Untouched Human T-Cells Kit (Life Technologies) according to manufacturer's instructions. Purified PBMCs were cultured in RPMI-media+10% heat inactivated FBS+1% Penicillin/Streptomycin in T-25 bulk flasks for 2-3 days alone or with SEB (Sigma-Aldrich) at 0.5 ng/mL (primary stimulation). At the end of the first round of SEB-stimulation, PBMCs were washed twice with PBS and immediately plated in 96-well tissue culture plates at a concentration of 1-5×10⁵ cells/well in media alone, media with a control antibody, media with SEB at 0.5 ng/mL (secondary stimulation) and no antibody, or media with SEB and DART A, a control IgG or an anti-PD-1 antibody+/−an anti-LAG-3 mAb, and cultured for an additional 2-3 days. At the end of the second stimulation, supernatants were harvested to measure cytokine secretion using human DuoSet ELISA Kits for IFNγ, TNFα, IL-10, and TL-4 (R&D Systems) according to the manufacturer's instructions.

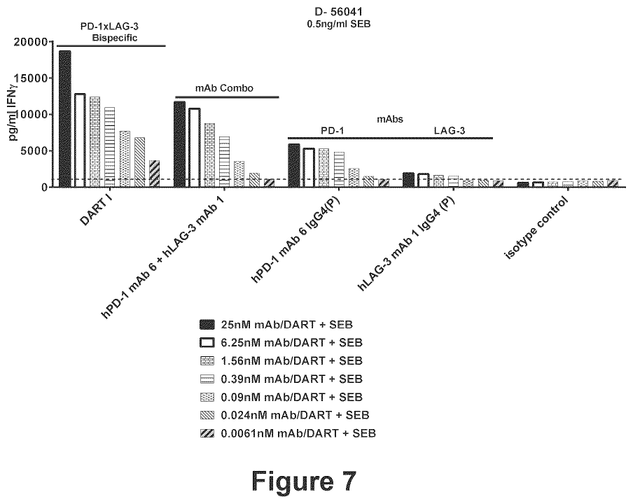

In these assays DART A (a PD-1×LAG-3 bispecific molecule) and the anti-PD-1 and anti-LAG-3 antibodies were used at a concentration of 0.0061, 0.024, 0.09, 0.39, 1.56, 6.25 or 25 nM. For these studies, where a combination of antibodies is used each antibody is provided at the indicated concentration and thus the total antibody concentration is twice the concentration used for each antibody (i.e., 0.0122, 0.048, 0.18, 0.78, 3.12, 12.5 or 50 nM). FIG. 7 shows the IFNγ secretion profiles from SEB-stimulated PBMCs from a representative donor (D: 56041). Similar results were seen for PD-1×LAG-3 bispecific molecules comprising VH/VL domains from alternative PD-1 and LAG-3 antibodies, and for PD-1×LAG-3 bispecific molecules have alternative structures (see, e.g., FIG. 3C, and for numerous donors.

The results of these studies demonstrate that PD-1×LAG-3 bispecific molecules dramatically enhanced IFNγ production from SEB-stimulated PBMCs upon restimulation. These results show that bispecific molecules that target two immunomodulatory pathways were more potent than the combination of separate antibodies targeting the same pathways.

### Example 2

### PD-1×CTLA-4 Bispecific Molecules

Bispecific molecules having specificity for PD-1 and CTLA-4 may be generated using methods provided herein and known in the art. The general structure of the polypeptide chains of several PD-1×CTLA-4 bispecific molecules is provided in Table 8. In particular, bispecific bivalent diabody molecules, comprising two polypeptide chains, having one binding site for PD-1 and one binding site for CTLA-4 may be generated wherein the polypeptide chains have the general structure of Variation I (also see, e.g., FIG. 1). Bispecific bivalent diabody molecules, comprising three polypeptide chains, having one binding site for PD-1, one binding site for CTLA-4 and an Fc Region may be generated wherein the polypeptide chains have the general structure of Variation II (also see, e.g., FIG. 4A). Bispecific tetravalent diabody molecules, comprising four polypeptide chains, having two identical binding sites for PD-1, two identical binding sites for CTLA-4 and an Fc Region may be generated wherein the polypeptide chains have the general structure of Variations III or IV (also see, e.g., FIGS. 3A-3C). In addition, bispecific trivalent molecules, comprising four polypeptide chains, having two binding sites for PD-1 and one binding site for CTLA-4 (or two binding sites for CTLA-4 and one binding site for PD-1), and an Fc Region may be generated wherein the polypeptide chains have the general structure of Variation V (also see, e.g., FIG. 6A). In addition, bispecific bivalent antibody molecules comprising four polypeptide chains having one binding site for PD-1, one binding site for CTLA-4 and an Fc Region may be generated wherein the polypeptide chains have the general structure of Variation VI (also see, e.g., U.S. Pat. No. 7,695,936 and PCT Patent Publication WO 2011/143545).

TABLE 8

| Vari- | Polypeptide | |
|---|---|---|
| ation | Chain | Domains |
| I | First | (VL1)-(Linker 1)-(VH2)-(Linker 2)-(HPD) |
| | Second | (VL2)-(Linker 1)-(VH1)-(Linker 2)-(HPD) |
| II | First | (VL1)-(Linker 1)-(VH2)-(Linker 2)-(HPD)- |
| | | (Linker 3)-(modified CH2—CH3 Domain) |
| | Second | (VL2)-(Linker 1)-(VH1)-(Linker 2)-(HPD) |
| | Third | (Linker3)-(modified CH2—CH3 Domain) |
| III | First and | (VL1)-(Linker 1)-(VH2)-(Linker 2)-(HPD)-(Linker |
| | Third | 3)-(CH2—CH3 Domain) |
| | Second and | (VL2)-(Linker 1)-(VH1)-(Linker 2)-(HPD) |
| | Fourth | |
| IV | First and | (VL1)-(Linker 1)-(VH2)-(Linker 2)-(CH1)-(Hinge)- |
| | Third | (CH2—CH3 Domain) |
| | Second and | (VL2)-(Linker 1)-(VH1)-(Linker 2)-(CL) |
| | Fourth | |
| V | First | (VL1)-(Linker 1)-(VH2)-(Linker 2)-(HPD)-(Linker |
| | | 3)-(modified CH2—CH3 Domain) |
| | Second | (VL2)-(Linker 1)-(VH1)-(Linker 2)-(HPD) |
| | Third | (VH3)—(CH1)-(Hinge)-(modified CH2—CH3 |
| | | Domain) |
| | Fourth | (VL3)—(CL) |
| VI | First | (VL1)-(Linker 1)-(VH2)-(Linker 2)-(HPD)-(Linker |
| | | 3)-(modified CH2—CH3 Domain) |
| | Second | (VL2)-(Linker 1)-(VH1)-(Linker 2)-(HPD) |
| | | (VL3)-(Linker 4)-(VH3)—(CH1)-(Hinge)-(modified |
| | Third | CH2—CH3 Domain) |
| VII | First | (VH1)—(CH1)-(Hinge)-(modified CH2—CH3 |
| | | Domain) |
| | Second | (VL1)—(CL) |
| | Third | (VH2)—(CH1)-(Hinge)-(modified CH2—CH3 |
| | | Domain) |
| | Fourth | (VL2)—(CL) |

HPD = Heterodimer-Promoting Domain

For each Variation of the bispecific molecules provided in Table 8:
(a) VL1 and VH1 are the variable domains of an anti-PD-1 antibody and VL2 and VH2 are the variable domains of an anti-CTLA-4 antibody; or
(b) VL1 and VH1 are the variable domains of an anti-CTLA-4 antibody and VL2 and VH2 are the variable domains of an anti-PD-1 antibody.
   For Variations V and VI: VL3 and VH3 are the variable domains of an anti-PD-1 antibody or are the variable domains of an anti-CTLA-4 antibody.

Linkers, Heterodimer-Promoting Domains and constant regions (e.g., CH1, Hinge, CH2-CH3 Domains) useful in the generation of such bispecific molecules are provided above. In particular, as detailed herein, for molecules whose first and third polypeptide chains are not identical the CH2-CH3 Domains are modified to promote heterodimerization and reduce or prevent homodimerization, for example by modifying the CH2-CH3 Domain one chain to comprise a “hole” and modifying the CH2-CH3 Domains on the other chain to comprise a “knob.” As detailed above, the Hinge and/or CH2-CH3 Domains may comprise amino acid substitutions, which stabilize the bispecific molecules and/or alter effector function and/or enhance serum half-life.

### Example 3

### Universal Bispecific Adaptor (“UBA”) Molecules

Alternatively, a bispecific molecule (e.g., a bispecific antibody, a bispecific diabody, trivalent binding molecule, etc.) may be constructed that comprises one epitope-binding site that specifically binds to PD-1 (or CTLA-4) and a second epitope-binding site that specifically binds a hapten, e.g. fluorescein isothiocyanate (also known as fluoroisothiocyanate or FITC). Such a bispecific molecule serves as a universal bispecific adaptor (“UBA”) molecule able to co-ligate a binding domain specific for PD-1 (or CTLA-4) with a fluorescein-conjugated binding molecule (e.g., an antibody, scFv, etc.) specific for CTLA-4 (or PD-1). For example, the FITC-reactive arm of such a universal bispecific adaptor molecule may be used to bind to a FITC labeled antibody that binds CTLA-4 (or PD-1) thereby generating a universal bispecific adaptor molecule that is adapted to bind PD-1 and CTLA-4. Such universal bispecific adaptor molecules are useful for the rapid assessment of bispecific molecules.

The anti-fluorescein antibody, 4-4-20 (“mAb 4-4-20”) may be employed as a source of FITC-specific binding domains (Gruber, M. et al. (1994) “Efficient Tumor CellLysis Mediated By A Bispecific Single Chain Antibody Expressed In Escherichia coli,” J. Immunol. 152(11): 5368-5374).

Amino Acid Sequence Of The Heavy Chain Variable Domain Of mAb 4-4-20 (SEQ ID NO:65) (CDR_{H} residues are underlined):

EVKLDETGGG LVQPGRPMKL SCVASGFTFS DYWMNWVRQS
PEKGLEWVAQ IRNKPYNYET YYSDSVKGRF TISRDDSKSS
VYLQMNNLRV EDMGIYYCTG SYYGMDYWGQ GTSVTVSS

Amino Acid Sequence Of The Light Chain Variable Domain Of mAb 4-4-20 (SEQ ID NO:66) (CDR_{L} residues are underlined):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGIKLE IK

Any of the bispecific formats provided herein may be utilized (see, e.g., Tables 1, 2, 3, and 4). Preferred bispecific molecules comprise only one hapten (e.g., fluorescein) binding site and will bind a single hapten-labeled antibody, thereby exhibiting a 1:1 ratio of universal adaptor bispecific molecule to hapten-labeled antibody in the resulting complexes. Such universal bispecific adaptor molecules may be constructed using, for example, the VL and VH Domains of an anti-PD-1 antibody and an anti-fluorescein antibody. Preferably, such a universal bispecific adaptor molecule is covalently bonded diabody or a trivalent binding molecule comprising two, three, four, five, or more polypeptide chains. Representative universal bispecific adaptor molecules which may be constructed are provided below.

A. UBA 1

One universal bispecific adaptor molecule that may be generated is a covalently bonded diabody composed of two polypeptide chains comprising one PD-1 epitope-binding site and one fluorescein binding site (“UBA 1”).

The first polypeptide chain of UBA 1 comprises, in the N-terminal to C-terminal direction, an N-terminus, the VL Domain of mAb 4-4-20 (SEQ ID NO:66), an intervening spacer peptide (Linker 1, GGGSGGGG (SEQ ID NO:5)), the VH Domain of PD-1 mAb 6 (SEQ ID NO:57, wherein X₁ is I)), an intervening spacer peptide (Linker 2, GGCGGG (SEQ ID NO:6)), the E-coil Heterodimer-Promoting Domain: EVAALEK-EVAALEK-EVAALEK-EVAALEK (SEQ ID NO:18)), and a C-terminus.

Thus, the amino acid sequence of the first polypeptide chain of UBA 1 is (SEQ ID NO:67):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGTKLE IKGGGSGGGG
QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSSG
GCGGGEVAAL EKEVAALEKE VAALEKEVAA LEK

The second polypeptide chain of UBA 1 comprises, in the N-terminal to C-terminal direction, an N-terminus, a VL Domain of PD-1 mAb 6 (SEQ ID NO:58, wherein X₁ is S and X₂ is Q)), an intervening spacer peptide (Linker 1, GGGSGGGG (SEQ ID NO:5)), the VH Domain of mAb 4-4-20 (SEQ ID NO:65)), an intervening spacer peptide (Linker 2, GGCGGG (SEQ ID NO:6)), the K-coil Heterodimer-Promoting Domain: KVAALKE-KVAALKE-KVAALKE-KVAALKE (SEQ ID NO:19)) and a C-terminus.

Thus, the amino acid sequence of the second polypeptide chain of UBA 1 is (SEQ ID NO:68):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGGSGGGG
EVKLDETGGG LVQPGRPMKL SCVASGFTFS DYWMNWVRQS
PEKGLEWVAQ IRNKPYNYET YYSDSVKGRF TISRDDSKSS
VYLQMNNLRV EDMGIYYCTG SYYGMDYWGQ GTSVTVSSGG
CGGGKVAALK EKVAALKEKV AALKEKVAAL KE

B. UBA 2

As provided above, incorporating an IgG CH2-CH3 Domains onto one polypeptide chain of a diabody such as UBA 1 will permit a more complex four-chain bispecific Fc Region-containing diabody to form. Thus a second universal bispecific adaptor molecule that may be generated is a covalently bonded diabody composed of four polypeptide chains comprising two PD-1 epitope-binding sites, two fluorescein binding sites, and an Fc Region (“UBA 2”). It will be noted that UBA 2 may bind two fluorescein labeled molecules via the two fluorescein binding sites.

The first and third polypeptide chains of UBA 2 comprises, in the N-terminal to C-terminal direction, an N-terminus, a VL Domain of a mAb 4-4-20 (SEQ ID NO:66), an intervening spacer peptide (Linker 1, GGGSGGGG (SEQ ID NO:5)), the VH Domain of PD-1 mAb 6 (SEQ ID NO:57, wherein X₁ is I)), an intervening spacer peptide (Linker 2, GGCGGG (SEQ ID NO:7)), the E-coil Heterodimer-Promoting Domain: EVAALEK-EVAALEK-EVAALEK-EVAALEK (SEQ ID NO:18)), an intervening spacer peptide (Linker 3, GGGDKTHTCPPCP (SEQ ID NO:31)), an IgG1 Fc Region comprising substitutions L234A/L235A (SEQ ID NO:43), wherein X is K), and a C-terminus.

Thus, the amino acid sequence of the first and third polypeptide chains of UBA 2 is (SEQ ID NO:69):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGTKLE IKGGGSGGGG
QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LTCLVKGFYP SDIAVEWESN
GQPENNYKTT PPVLDSDGSF FLYSKLTVDK SRWQQGNVFS
CSVMHEALHN HYTQKSLSLS PGK

The second and fourth polypeptide chains of UBA 2 are identical to the second polypeptide chain of UBA 1. Thus, the second and fourth polypeptide chains of UBA 2 each have the amino acid sequence of SEQ ID NO:68.

C. UBA 3

A third universal bispecific adaptor molecule that may be generated is a covalently bonded diabody composed of three polypeptide chains comprising one PD-1 epitope-binding site, one fluorescein binding site, and an Fc Region (“UBA 3”).

The first polypeptide chain of UBA 3 comprises, in the N-terminal to C-terminal direction, an N-terminus, the VL Domain of mAb 4-4-20 (SEQ ID NO:66), an intervening spacer peptide (Linker 1, GGGSGGGG (SEQ ID NO:5)), the VH Domain of PD-1 mAb 6 (SEQ ID NO:57, wherein X₁ is I)), an intervening spacer peptide (Linker 2, GGCGGG (SEQ ID NO:6)), the E-coil Heterodimer-Promoting Domain: EVAALEK-EVAALEK-EVAALEK-EVAALEK (SEQ ID NO:18)), an intervening spacer peptide (Linker 3, GGGDKTHTCPPCP (SEQ ID NO:31)), a “knob-bearing” IgG1 Fc Region comprising substitutions L234A/L235A (SEQ ID NO:44, wherein X is K)), and a C-terminus.

Thus, the amino acid sequence of the first polypeptide chain of UBA 3 is (SEQ ID NO:70):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGTKLE IKGGGSGGGG
QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSSG
GCGGGEVAAL EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

The second polypeptide chain of UBA 3 may be identical to the second polypeptide chain of UBA 1. Thus, the second polypeptide chain of UBA 3 has the amino acid sequence of SEQ ID NO:68.

The third polypeptide chains of UBA 3 comprises, in the N-terminal to C-terminal direction, an N-terminus, a spacer peptide (Linker 3, DKTHTCPPCP (SEQ ID NO:26)), a “hole-bearing” IgG1 Fc Region comprising substitutions L234A/L235A (SEQ ID NO:45, wherein X is K)), and a C-terminus.

Thus, the amino acid sequence of the third polypeptide chain of UBA 3 is (SEQ ID NO:71).

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

D. UBA 4

A fourth universal bispecific adaptor molecule that may be generated is a covalently bonded trivalent binding molecule composed of four polypeptide chains comprising two PD-1 epitope-binding sites, one fluorescein binding site, and an Fc Region (“UBA 4”).

The first polypeptide chain of UBA 4 is identical to the first polypeptide chain of UBA 3. Thus, the first polypeptide chains of UBA 4 has the amino acid sequence of SEQ ID NO:70.

The second polypeptide chain of UBA 4 is identical to the second polypeptide chain of UBA 1. Thus, the second polypeptide chain of UBA 4 has the amino acid sequence of SEQ ID NO:68.

The third polypeptide chain of UBA 4 comprises, in the N-terminal to C-terminal direction, the VH Domain of PD-1 mAb 6 (SEQ ID NO:57, wherein X₁ is I)), an IgG1 CH1 Domain (SEQ ID NO:40), an IgG1 Hinge Region (SEQ ID NO:33), a “hole-bearing” IgG1 Fc Region comprising substitutions L234A/L235A (SEQ ID NO:45, wherein X is K)), and a C-terminus.

Thus, the amino acid sequence of the third polypeptide chain of UBA 4 is (SEQ ID NO:72):

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSSA
STKGPSVFPL APSSKSTSGG TAALGCLVKD YFPEPVTVSW
NSGALTSGVH TFPAVLQSSG LYSLSSVVTV PSSSLGTQTY
ICNVNHKPSN TKVDKRVEPK SCDKTHTCPP CPAPEAAGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSREEM
TKNQVSLSCA VKGFYPSDIA VEWESNGQPE NNYKTTPPVL
DSDGSFFLVS KLTVDKSRWQ QGNVFSCSVM HEALHNRYTQ
KSLSLSPGK

The fourth polypeptide chain of UBA 4 comprises, in the N-terminal to C-terminal direction, the VL Domain of PD-1 mAb 6 (SEQ ID NO:58, wherein X₁ is S and X₂ is Q)), a CL Domain (e.g., an IgG Kappa Domain (SEQ ID NO:38), and a C-terminus.

Thus, the amino acid sequence of the fourth polypeptide chain of UBA 4 is (SEQ ID NO:73):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

E. UBA 5

A fifth universal bispecific adaptor molecule that may be generated is a covalently bonded trivalent binding molecule composed of three polypeptide chains comprising two PD-1 epitope-binding sites, one fluorescein binding site, and an Fc Region (“UBA 4”) (see, e.g., FIG. 6C-6D).

The first polypeptide chain of UBA 5 is identical to the first polypeptide chain of UBA 3. Thus, the first polypeptide chains of UBA 5 has the amino acid sequence of SEQ ID NO:70.

The second polypeptide chain of UBA 5 is identical to the second polypeptide chain of UBA 1. Thus, the second polypeptide chain of UBA 5 has the amino acid sequence of SEQ ID NO:68.

The third polypeptide chain of UBA 5 comprises, in the N-terminal to C-terminal direction, the VL Domain of PD-1 mAb 6 (SEQ ID NO:58, wherein X₁ is S and X₂ is Q)), an intervening spacer peptide (Linker 4, GGGGSGGGGSGGGGS (SEQ ID NO:37)), the VH Domain of PD-1 mAb 6 (SEQ ID NO:57, wherein X₁ is I)), an IgG1 CH1 Domain (SEQ ID NO:40), an IgG1 Hinge Region (SEQ ID NO:33), a “hole-bearing” IgG1 Fc Region comprising substitutions L234A/L235A (SEQ ID NO:45, wherein X is K)), and a C-terminus.

Thus, the amino acid sequence of the third polypeptide chain of UBA 5 is (SEQ ID NO:74):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGGSGGGG
SGGGGSQVQL VQSGAEVKKP GASVKVSCKA SGYSFTSYWM
NWVRQAPGQG LEWIGVIHPS DSETWLDQKF KDRVTITVDK
STSTAYMELS SLRSEDTAVY YCAREHYGTS PFAYWGQGTL
VTVSSASTKG PSVFPLAPSS KSTSGGTAAL GCLVKDYFPE
PVTVSWNSGA LTSGVHTFPA VLQSSGLYSL SSVVTVPSSS
LGTQTYICNV NHKPSNTKVD KRVEPKSCDK THTCPPCPAP
EAAGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSREEMTKNQ VSLSCAVKGF YPSDIAVEWE SNGQPENNYK
TTPPVLDSDG SFFLVSKLTV DKSRWQQGNV FSCSVMHEAL
HNRYTQKSLS LSPGK

Using conventional methods, anti-CTLA-4 antibodies may be labeled with fluorescein. When such labeled molecules are incubated in the presence of a universal bispecific adaptor molecule provided above having an epitope-binding site that binds to PD-1 and an epitope-binding site that binds to fluorescein, they form a PD-1×CTLA-4 bispecific molecule, which may be assayed as described below.

It will be appreciated in view of the teachings provided herein that different VH Domains, VL Domains, linkers, heterodimer promoting domains, and/or IgG Constant Domains could be utilized to generate alternative universal bispecific adaptor molecules. For example, the VH and VL Domains of an anti-CTLA-4 antibody and/or a different anti-PD-1 antibody could be used in place of the VH and VL Domains of the employed anti-PD-1 antibody to generate alternative or equivalent universal bispecific adaptor molecules. Alternatively, the VH and VL Domains of an anti-CTLA-4 antibody may be used in place of the VH and VL Domains of the anti-fluorescein antibody to generate PD-1×CTLA-4 bispecific molecules having the general structure of Variations I, II, III, V and VI provided above. Such PD-1×CTLA-4 bispecific molecules may be used directly in the assays described below.

### Example 4

### Assays

The PD-1×CTLA-4 bispecific molecules of the present invention may be characterized in any of a variety of ways. In particular, PD-1×CTLA-4 bispecific molecules of the invention may be assayed for their ability to immunospecifically bind to the PD-1 and CTLA-4 molecules (e.g., as present on a cell surface, etc.), and/or the binding kinetics of the interactions with antigen may be determined. Where the bispecific molecules comprise an Fc region (or portion thereof), their ability to exhibit Fc-FcγR interactions, e.g., specific binding of an Fc region (or portion thereof) to an FcγR, mediation of effector function, signal transduction, etc., may be assayed. The immunomodulatory activity and/or in vivo anti-tumor efficacy of the PD-1×CTLA-4 bispecific molecules of the invention may be assayed using in vitro and in vivo assays known in the art.

A. Preparation of Immune Cells and Cell Expressing PD-1 and/or CTLA-4

1. Isolation of PBMCs and Immune Cell Subpopulations from Human Whole Blood

PBMCs from healthy human donors are isolated from whole blood, for example, using Ficoll gradient centrifugation. Briefly, whole blood is diluted 1:1 with sterile phosphate buffered saline (PBS). The diluted blood (35 mL) is layered onto 15 mL of Ficoll-Paque™ Plus in a 50 mL tube and the tubes are centrifuged at 400×g (1320 rpm) for 30 minutes with the brake off. The buffy-coat layer between the two phases is collected into 50 mL tubes and centrifuged at 600×g (1620 rpm) for 5 minutes. The supernatant is discarded and the cell pellet is washed 3 times with PBS (e.g., by centrifuging the tubes at 600×g (1620 rpm) for 5 minutes). Viable cell count is determined using Trypan Blue dye. The PBMCs are resuspended in complete culture medium (e.g., RPMI 1640, 10% FBS, 1% pen/strep) and incubated at 37° C. with 5% CO₂ overnight or are further processed to isolate a desired immune cell subpopulation such as T cells, (e.g., T regs, CD8, CD4), NK cells, dendritic cells and monocytes as described below.

Particular immune cell subpopulations are readily isolated from PBMCs using a commercial preparation kit (e.g., the Untouched™ human T cell isolation kits for isolation of T-cells, CD4 T-cells, CD8 T-cells, Monocytes, Dendritic Cells (Life Technologies/ThermoFisher Scientific); the DYNABEADS® Regulatory CD4+/CD35+ T Cell Kit for isolation of T regulatory cells (CD4+/CD25+) (ThermoFisher), etc.), according to the manufacturer's instructions. After isolation, the immune cell subpopulation (e.g., T cells) are resuspended in the appropriate complete culture medium (e.g., RPMI 1640, 10% FBS, 1% penicillin/streptomycin, which may be supplemented with cytokines (e.g., IL-2, GM-CF, IL-4, TNF-α, etc.) and incubated at 37° C. with 5% CO₂ overnight. As provided herein such purified subpopulations are useful to evaluate cell surface expression of PD-1 and/or CTLA-4 and for evaluation of the immune stimulatory activity of the PD-1×CTLA-4 bispecific molecules of the invention.

2. Isolation of PBMCs from Cynomolgus Monkey or Rhesus Monkey Whole Blood

PMBCs from Cynomolgus monkey or Rhesus monkey are isolated from whole blood, for example using Ficoll gradient centrifugation. Briefly, whole blood is diluted 1:3 with sterile PBS. Diluted blood (35 mL) is layered onto 15 mL of 90% Ficoll-Paque™ Plus (90 mL Ficoll+10 mL PBS) in a 50 mL polypropylene centrifuge tube and centrifuged at 931×g (2000 rpm) for 30 minutes at room temperature with the brake off. The buffy-coat layer between the two phases is collected and transferred to a clean 50 mL tube and washed with 45 mL PBS by centrifuging the tubes at 600×g (1620 rpm) for 5 minutes. The supernatant is discarded and the pellet is rinsed 3× with PBS. Cynomolgus or Rhesus monkey PBMCs are then resuspended in 30 mL of complete culture medium and viable cell count is determined by Trypan Blue dye exclusion.

Particular immune cell subpopulations are readily isolated from non-human primate PBMCs using a commercial preparation kit (e.g., Pan T-cell, CD4+ T-Cell, and CD4+/CD25+ Treg isolation kits (Miltenyl Biotech)), according to the manufacturer's instructions. Alternatively, flow cytometric sorting using non-human primate specific or cross-reactive mAbs can be used for sorting.

3. Generation of Human Immature or Mature Myeloid-Derived Dendritic Cells (mDC) Cells from Isolated Human Monocytes

Human monocytes are isolated from donor derived purified PBMCs using a commercial preparation kit (e.g., the Untouched™ human monocyte kit (Life Technologies/ThermoFisher Scientific) according to manufacturer's instructions. Isolated human monocytes are induced to differentiate into human immature mDCs by culturing monocytes (e.g., in alpha Minimum Essential Media with nucleosides (αMEM) media+2% human AB-negative serum+1% penicillin/streptomycin) for 5-7 days in the presence of recombinant human granulocyte macrophage-colony stimulating factor (e.g., hGM-CSF; Peprotech, 100 ng/ml) and recombinant human interleukin-4 (hIL-4; Peprotech, 40 ng/ml). Immature mDCs are harvested and washed with PBS by centrifuging the tubes at 600×g (1620 rpm) for 5 minutes for use as stimulator cells in allogeneic mixed lymphocyte reaction (allo-MLR) assays, such as those detailed below.

In certain allo-MLR experiments immature mDCs are induced to differentiate by adding TNFα or a cocktail of additional cytokines (IFNγ, IL-1β) and mitogens (LPS) for two additional days of culture (see, e.g., Han, T. (2009) “Evaluation of 3 Clinical Dendritic Cell Maturation Protocols Containing LPS and IFN-gamma,” J Immunother 32:399). The purity, maturation and activation of mDCs may be evaluated by flow cytometry using one or more of the following antibodies: anti-CD14, anti-CD80, anti-CD83, anti-CD86, anti-HLA-DR; and the appropriate isotype controls. The flow cytometric data from such evaluations may be acquired on a FACSCalibur/Fortessa (Becton Dickinson/BD Biosciences) and analyzed using FlowJo software (TreeStar).

4. Expression of PD-1 and CTLA-4

Cells expressing PD-1 and/or CTLA-4 may be generated using methods known in the art. For example, cells (e.g., NSO, Jurkat, CHO, etc.) may be engineered to express PD-1 and/or CTLA-4 using retroviral vectors containing the appropriate gene (e.g., human PD-1 gene). Alternatively, immune cells may be stimulated to induce or increase the expression of PD-1 and/or CTLA-4. Briefly, purified immune cells (e.g., PBMCs, T-cells, dendritic cells, etc.) isolated as described above are cultured for 2-6 days in the presence or absence of a mitogen and the expression of PD-1 and/or CTLA-4 is examined on the untreated (Naïve) and stimulated cells, for example using flow cytometry. Commercial anti-PD-1 and anti-CTLA-4 antibodies can be used for preliminary evaluation of the expression patterns on Naïve cells and in response to mitogen stimulation. Additionally, or optionally the PD-1×CTLA-4 bispecific molecules of the invention may be used.

Mitogens which may be utilized for such studies are well known in the art and include, but are not limited to: CD3/CD28 beads, lipopolysaccharides (LPS), Staphylococcus aureus enterotoxin types A-E (e.g., SEB), phorbol myristate acetate (PMA), phytohemagglutinin (PHA), concanavalin A (conA), pokeweed mitogen (PWM), etc. Mitogen(s) identified as inducing/enhancing the expression of PD-1 and/or CTLA-4 may be used in functional assays to evaluate the stimulatory activity of the PD-1×CTLA-4 bispecific molecules of the present invention. See for example the “SEB”, and “MLR” assays described herein.

B. Binding Assays

Immunoassays that can be used to analyze immunospecific binding to PD-1 or CTLA-4 molecules, binding cross-reactivity, or Fc-FcγR interactions include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunochromatographic assays, immunodiffusion assays, agglutination assays, complement-fixation assays, immunoradiometric assays, fluorescent immunoassays, and protein A immunoassays, etc. (see, e.g., Ausubel et al., 2008, Current Protocols in Molecular Biology). Binding affinity for a target antigen is typically measured or determined by standard antibody-antigen assays, such as Biacore competitive assays, saturation assays, or immunoassays such as ELISA or RIA. Fluorescence activated cell sorting (FACS), using any of the techniques known to those skilled in the art, is used for immunological or functional based assays to characterize the PD-1×CTLA-4 bispecific molecules of the invention.

For example, PBMCs may be prepared as described above. Where desired immune cell subsets (e.g., T regulatory, T helper, APCs, etc.) may be isolated from the purified PBMC. The isolated cells are then examined for PD-1 and CTLA-4 expression on various cell subsets (e.g., T regulatory, T helper, APCs, etc.) by co-staining and FACS analysis as described below.

1. Cell Surface Binding (Saturation Assay)

The ability of PD-1×CTLA-4 bispecific molecules to bind to PD-1 and/or CTLA-4 expressed on the cell surface may be measured in saturation/dilution based assays using a cell that expresses PD-1 and/or CTLA-4 (target cells). Such cells may be immune cells stimulated to expressed PD-1 and/or CTLA-4, or a cell line (e.g., NSO cells) engineered to stably over-express PD-1 and/or CTLA-4 molecules. Briefly, cultured targets cells (e.g., NSO cell engineered to express PD1⁺) are harvested and resuspended (e.g., about 5×10⁶ cells/ml) in blocking buffer (e.g., FACS buffer+10% human AB Serum). Starting at equal molar concentrations (e.g., 20 nM in total of 200 μl) a PD-1×CTLA-4 bispecific molecule, an anti-PD-1 antibody, an anti-CTLA-4 or a combination of anti-PD-1 and anti-CTLA-4 antibodies are prepared for dilution in a separate microtiter plate and then serially diluted (e.g., 1:4, 1:5, 1:10, etc.) 5-12 times to generate a 5-12 point curve. The highest starting concentration in all experiments is determined empirically. The same volume (e.g., 50 μl) of each dilution is added to a new microtiter plate and target cells are added to each well (e.g., 0.25×10⁶ cells/well) and incubated (e.g., at 4-25° C. for 30-120 minutes). The cells are washed 1-3 times (e.g., the microtiter plate is spun at 600×g (1620 rpm) for 5 minutes and then washed with blocking buffer and spun again) and resuspended in blocking buffer. For secondary staining, the appropriate secondary regent is selected, for example a goat anti-Human Fc-APC may be used to detect human primary antibodies, while a goat Anti-Mouse IgG Fc Alexa Fluor 647 is used to detect mouse primary antibodies. The selected secondary reagent is diluted in blocking buffer and based on the concentration of the individual secondary, a stock solution is made and the same volume/well of the secondary mixture is aliquoted to individual wells and incubated (e.g., at 4-25° C. 30-120 minutes). The cells are washed as described above and resuspended in blocking buffer. The stained cells are analyzed by flow cytometry. The flow cytometric data may be acquired on a FACSCalibur/Fortessa (Becton Dickinson/Fortessa), analyzed as mean fluorescent intensity using FlowJo software (TreeStar), and plotted and fitted using the log(agonist) vs. response-variable slope (four parameter) function in Prism6 software (Graphpad).

2. Receptor/Ligand Binding and Signaling Assays

Assays that can be used to analyze the ability of the PD-1×CTLA-4 bispecific molecules of the invention to modulate (e.g., block, inhibit, stimulate, etc.) ligand binding and signaling are provided in more detail below.

a. PD-1 Receptor/Ligand Binding

The ability of PD-1×CTLA-4 bispecific molecules to inhibit PD-1 from binding PD-L1 and/or PD-L2 may be evaluated using cells that express PD-1 (target cells). Such cells may be immune cells stimulated to express PD-1, or a cell line engineered to express PD-1 molecule, for example NSO-cells retrovirally transduced with the human PD-1 gene. Briefly, PD-1 expressing cells (e.g., NSO/PDCD1 (NSO-PD1⁺)) are harvested and resuspended (e.g., about 1.5×10⁶ cells/ml) in blocking buffer (e.g., FACS buffer+10% Human Ab Serum) and plated in a microtiter plate (e.g., 0.25×10⁶ cells/well). Starting at equal molar concentrations (e.g., 20 nM in total of 200 μl) of a PD-1×CTLA-4 bispecific molecule, an anti-PD-1 antibody, an anti-CTLA-4, or a combination of anti-PD-1 and anti-CTLA-4 antibodies are prepared for dilution in a separate microtiter plate and serially diluted (e.g., 1:4, 1:5, 1:10, etc.) 5-12 times to generate a 5-12 point curve. The highest starting concentration in all experiments is determined empirically. The same volume (e.g., 50 μl) of each dilution is added to each well of the microtiter plate containing the target cells. To evaluate the inhibition of PD-L1 binding a soluble PD-L1 fusion protein (e.g., hPD-L1 (B7H1) TEV-hIgG1-Fc-biotin (Ancell)) is added to each well with the exception of unstained negative control wells and incubated (e.g., at 4-25° C. for 30-120 minutes). To evaluate the inhibition of PD-L2 binding a soluble PD-L2 fusion protein (e.g., CD273 (PD-L2) muIgG/biotin (Ancell)) is added to each well with the exception of unstained negative control wells and incubated (e.g., at 4-25° C. for 30-120 minutes). The cells are washed 1-3 times (e.g., the microtiter plate is spun at 600×g (1620 rpm) for 5 minutes and then washed with blocking buffer and spun again). The cells are resuspended in blocking buffer. With the exception of unstained negative control wells, the appropriate secondary reagent for detection of the PD-L1 or PD-L2 fusion protein (e.g., streptavidin-PE labeled secondary (eBiosciences)) is added and incubated (e.g., at 4-25° C. for 15-120 minutes). The cells are washed as described above and resuspended in blocking buffer. The stained cells may be analyzed by flow cytometry. The flow cytometric data may be acquired on a FACSCalibur/Fortessa (Becton Dickinson/Fortessa), and analyzed for the loss mean fluorescent intensity of labeled sPD-L1 or sPD-L2 in the presence of a PD-1×CTLA-4 bispecific molecule, an anti-PD-1 antibody, an anti-CTLA-4, or a combination of anti-PD-1 and anti-CTLA-4 antibodies using FlowJo software (TreeStar), and plotted and fitted using the log(agonist) vs. response-variable slope (four parameter) function in Prism6 software (Graphpad).

b. CTLA-4 Receptor/Ligand Binding

The ability of PD-1×CTLA-4 bispecific molecules to inhibit CTLA-4 from binding CD80 and/or CD86 may be evaluated using cells that express CTLA-4 (target cells). Such cells may be immune cells stimulated to express CTLA-4, or a cell line engineered to express CTLA-4, for example NSO-cells retrovirally transduced with the human CTLA-4 gene. Briefly, CTLA-4 expressing cells are harvested and resuspended in blocking buffer (e.g., FACS buffer+10% Human Ab Serum) and plated in a microtiter plate (e.g., 0.25×10⁶-1.0×10⁶ cells/well). Starting at equal molar concentrations (e.g., 20 nM in total of 200 μl) of a PD-1×CTLA-4 bispecific molecule, an anti-PD-1 antibody, an anti-CTLA-4, or a combination of anti-PD-1 and anti-CTLA-4 antibodies are prepared for dilution in a separate microtiter plate and serially diluted (e.g., 1:4, 1:5, 1:10, etc.) 5-12 times to generate a 5-12 point curve. The highest starting concentration in all experiments is determined empirically. The same volume (e.g., 50 μl) of each dilution is added to each well of the microtiter plate containing the target cells. To evaluate the inhibition of CD80 binding a soluble CD80 fusion protein (e.g., hCD80-muIg-biotin (ADIPOGEN®)) is added to each well with the exception of unstained negative control wells and incubated (e.g., at 4-25° C. for 30-120 minutes). To evaluate the inhibition of CD86 binding a soluble CD86 fusion protein (e.g., hCD86-mulg-biotin (ADIPOGEN®)) is added to each well with the exception of unstained negative control wells and incubated (e.g., at 4-25° C. for 30-120 minutes). The cells are washed 1-3 times (e.g., the microtiter plate is spun at 600×g (1620 rpm) for 5 minutes and then washed with blocking buffer and spun again). The cells are resuspended in blocking buffer. With the exception of unstained negative control wells, the appropriate secondary reagent for detection of the CD80 or CD86 fusion protein (e.g., streptavidin-PE labeled secondary (eBiosciences)) is added and incubated (e.g., at 4-25° C. for 15-120 minutes). The cells are washed as described above and resuspended in blocking buffer. The stained cells may be analyzed by flow cytometry. The flow cytometric data may be acquired on a FACSCalibur/Fortessa (Becton Dickinson/Fortessa), and analyzed for the loss mean fluorescent intensity of labeled CD86 or CD80 in the presence of a PD-1×CTLA-4 bispecific molecule, an anti-PD-1 antibody, an anti-CTLA-4, or a combination of anti-PD-1 and anti-CTLA-4 antibodies using FlowJo software (TreeStar), and plotted and fitted using the log(agonist) vs. response-variable slope (four parameter) function in Prism6 software (Graphpad).

C. Reporter Assays

The functional activity of PD-1×CTLA-4 bispecific molecules in blocking the interaction of PD-1 with PD-L1 may be assessed using a commercial reporter system developed by Promega according to the manufacturer's direction. Briefly, two cell lines engineered to function as either a stimulator line or reporter cell line are used. The stimulator line was engineered from a CHO-parental line to express the PD-L1 molecule and a T cell activator, which is a membrane bound anti-CD3 agonist mAb [CHO/PDL1 cells]. The reporter cell line was engineered from a CD3-positive Jurkat parental line to express a luciferase reporter construct under the transcription control of nuclear factor of activated T-cells (NFAT) [NFAT-luc2/PD-1 Jurkat cells]. When cultured together, the anti-CD3 agonist expressed on the CHO-PDL1 cell line drives luciferase expression by the NFAT signal transduction pathway mediated by the engagement of the TCR/CD3 signaling complex present on the Jurkat-NFAT-luc/PD-1 cell line. In the absence of anti-PD-1 or anti-PD-L1 antibodies, luciferase is expressed at a level relative to TCR/CD3 signaling but down-modulated or inhibited by the presence of the PD-1/PD-L1 inhibitory axis, which functions as a brake. In the presence of molecules which inhibit PD-1/PD-L1 signaling (e.g., anti-PD-1 or anti-PD-L1 antibodies), this inhibitory axis or “brake” is released, permitting enhanced luciferase expression that can be measured. Accordingly, the PD-1 inhibitory activity of PD-1×CTLA-4 bispecific molecules may be evaluated by culturing CHO/PDL1 with NFAT-luc2/PD1 Jurkat (3H-D5). Briefly, CHO-PDL1 are plated into a microtiter plate (e.g., at 4.0×10⁴ cells/well) and cultured overnight (e.g., in RPMI media containing 10% FBS+100 ug/mL Hygromycin B+500 ug/mL G418). The next day, assay buffer (e.g., RPMI+2% FBS is prepared along with a 5-12 point serial dilution of a PD-1×CTLA-4 bispecific molecule, or an anti-PD-1 antibody in assay buffer with highest dilution point at equal molar equivalence (e.g., 100-200 nM) and 5-12 serial dilutions (e.g., 1:4, 1:5, 1:10, etc.) are prepared. In the following order, a portion of cell the culture media is removed from the microtiter plate containing adherent CHO/PDL1 cells and aliquots of each dilution are added to the CHO/PDL1 cells. Cultured NFAT-luc2/PD-1 Jurkat cells are harvested and resuspended in assay buffer and added (e.g., 5.0×10⁴ cells/well in 40 μl/well) to the CHO/PDL1 cells. The co-culture is incubated (e.g., for 6 hours at 37° C.). At the end of the incubation, Bio-Glo substrate (Promega) is reconstituted and added to the ambient temperature equilibrated microtiter plate. Following incubation (e.g., 5-10 minutes) the optical density of each well is read on a VICTOR™ X4 Multilabel Plate Reader (Perkin Elmer #2030-0040) at 450 nm with luminescence relative light unit (RLU) as the readout. The data may then be plotted and fitted using the log(agonist) vs. response-variable slope (four parameter) function in Prism6 software (Graphpad).

Similar reporter assays are available for CTLA-4 signaling (e.g., CTLA-4 Blockade Bioassay Kit (Promega)) and/or may be readily generated to analyze the functional activity of PD-1×CTLA-4 bispecific molecules in blocking the interaction CTLA-4 with its respective ligand(s).

D. Immunomodulatory Assays

Assays that can be used to analyze the immunomodulatory activity of the PD-1×CTLA-4 bispecific molecules of the invention include mitogen stimulation assays such as the “SEB” assay detailed above, and Mixed Lymphocyte Reaction (MLR) assays such as those provided in more detail below. The ability of the PD-1×CTLA-4 bispecific molecules of the invention to modulate both the PD-1 and the CTLA-4 inhibition pathways is expected to provide enhanced stimulation in assays as compared to anti-PD1 and anti-CTLA-4 antibodies alone or the combination of such antibodies.

PBMCs or T cells are isolated from the blood of the same (autologous) or unrelated (allogeneic) patient(s) healthy donor(s) blood by centrifugation over a Ficoll-Paque™ gradient as described above and resuspended in complete culture medium. For allo-MLR assays that employ mDCs, monocytes are purified and matured as describe above. For one-way (unidirectional) allo-MLR assays responder cells (e.g., PBMCs) are co-cultured with stimulating cells in a microtiter plate. Depending on the context, stimulating cells are DCs, autologous PBMCs (for auto-MLR, i.e., negative control), or allogeneic PBMCs (for allo-MLR, i.e., positive control). The ratio of responder:stimulating cells is typically 1:1 or 2:1, but may be varied. The co-cultures are performed in the presence of equal molar amounts of serial (e.g., 1:4 1:5, 1:10, etc.) dilutions of a PD-1×CTLA-4 bispecific molecule, an anti-PD-1 antibody, an anti-CTLA-4, a combination of anti-PD-1 and anti-CTLA-4 antibodies, or the corresponding isotype mAbs. Serial antibody dilutions may be prepared as described above. In addition, single cell populations controls stimulated with or without anti-CD3+/−anti-CD28 mAbs may be used as controls in such experiments. Stimulating cells (stimulators) are pre-irradiated (e.g., at 45 grays[Gy] (4500 rads) using a Gammacell® 3000 Elan Blood/Cell Irradiator (Theratronics)) to prevent proliferation of the stimulator cells and allow measurement of only the proliferation of the responding cell (responders). After 5-7 days (the time will be adjusted to ensure expression of PD-1 and CTLA-4 during the assay), [3H]-thymidine (e.g., 1 Ci/well (Perkin Elmer)) is added for further 18-48 hours. The radioactivity incorporated into DNA is measured in (e.g., in a TOPCountNXT β-scintillation counter (Perkin Elmer)). Results are expressed as either mean counts per minute (cpm) or expressed as stimulation index (SI) allowing the comparison of results from different donors. SI is calculated as follows: mean counts per minute (cpm) from stimulated cells divided by mean cpm from non-stimulated cells. MLR responses are considered positive when SI was ≥3 for PBMC-induced stimulation and SI≥6 for DC-induced stimulation. Alternatively, proliferation may be measured, using a CEFSE-based proliferation assay (Boks, M. A., et al. (2010) “An optimized CFSE based T-cell suppression assay to evaluate the suppressive capacity of regulatory T-cells induced by human tolerogenic dendritic cells,” Scand J Immunol 72:158-168).

Additional MLR assays which may be used to evaluate the immune stimulatory activity of the PD-1×CTLA-4 bispecific molecules of the invention are known in the art. See, for example, Davies, J. K. et al. (2011) “Induction of alloantigen-specific anergy in human peripheral blood mononuclear cells by alloantigen stimulation with co-stimulatory signal blockade,” Journal of Visualized Experiments: JoVE, (49), 2673; Kruisbeek, A. M., et al. (2004) “Proliferative Assays for T cell Function,” CURRENT PROTOCOLS IN IMMUNOLOGY, 60:11:3.12.1-3.12.20; Wallgren, A. C. et al. (2006) “The Direct Pathway Of Human T-Cell Allorecognition Is Not Tolerized By Stimulation With Allogeneic Peripheral Blood Mononuclear Cells Irradiates With High-Dose Ultraviolet,” Ba. Scand J of Immunol 63:90-96; Levitsky, J. et al. (2009) “The Human ‘Treg MLR’ Immune Monitoring for Foxp3+ T regulatory cell generation, Transplantation 88:1303-11.

E. In Vivo Anti-Tumor Assays

The anti-tumor activity of the PD-1×CTLA-4 bispecific molecules of the invention may be evaluated in various animal models known in the art. Treatment with the PD-1×CTLA-4 bispecific molecules of the invention is expected to inhibit tumor establishment and/or tumor growth to a greater extent than treatment with anti-PD1 and anti-CTLA-4 antibodies alone or the combination of such antibodies.

Murine xenograph tumor models are particularly useful. Briefly, mice are implanted with a cancer cell line, or tumor cells of interest and are treated with (i) a PD-1×CTLA-4 bispecific molecule (ii) an anti-PD-1 antibody (iii) an anti-CTLA-4 antibody (iv) a combination of anti-PD-1 and anti-CTLA-4 antibody, and (vi) no-treatment control which may be vehicle alone and/or an irrelevant antibody. Treatment may begin prior to implantation (e.g., 1 day before (i.e., day −1)); on the same day as implantation (i.e., day 0), or after establishment of a tumor (e.g., day 7). The animals may receive a single treatment or may receive multiple treatments (e.g., weekly post implantation). The animals are monitored over time to determine the in vivo effect of these molecules on tumor establishment and/or growth. Growth of tumors may be monitored my measuring the tumors and determining the tumor volume (height×width×length). Treated animals which show complete tumor regression can be used to examine tumor-specific immunity by rechallenge using the same or tumor cells and irrelevant tumor cells as a control. In addition, these models may be modified to include combination treatment with standard of care treatments such as chemotherapy, radiation, etc.

Numerous transplantable cancer cell lines which may be utilized in such xenograph models are known in the art and include, but are not limited to: MDST8, SW480 and SW620 colorectal cancer cells; AGS gastric cancer cells; UACC-62, A2058, and LOX IN4VI melanoma cells; 22rv prostate cancer cells; AsPC-1 and BxPc-3 pancreatic cancer cells; Caki-1, A498 and 786-0 renal cancer cells; HT-1197 Bladder cancer cells; 4T1, MDA-MB-231, mammary cancer cells; A549, WX322 Lung cancer cells; HT1080 Fibrosarcoma cells; HBL-2 human mantle cell lymphoma cells; Raji Burkitt's lymphoma cells. Particularly preferred are Patient-Derived Xenograft (PDX) models. Such cancer cell lines, or patient-derived tumors are engrafted into immunocompromised mice strains (e.g., Nude mice, Scid mice, NOD mice, Rag 1 null mice, etc. (see, e.g., Belizario, J. E., (2009) “Immunodeficient Mouse Models: An Overview,” Bentham Open 1874-2262/09) or humanized mice such as transgenic human HLA-A2 mice (see, e.g., Shultz, L. D., et al. (2012) “Humanized mice for immune system investigation: progress, promise and challenges,” Nature Rev Immunol 12:786-798) as described above. In addition, for evaluation of molecules which modulate immune checkpoint immune-deficient mice may be engrafted with human immune system components (e.g., reconstituted with human PBMCs, stem cells, immune progenitor cells, etc.) prior to or concurrently with implantation of the desired tumor cells and treatment as detailed above.

### Example 5

### PD-1×CTLA-4 Bispecific Molecules Binding Studies

Several PD-1×CTLA-4 bispecific molecules were generated, including Fc Region-containing diabodies and Fc-Region-containing trivalent molecules comprising four polypeptides chains. Three diabodies having four polypeptide chains and comprising E/K-coil Heterodimer-Promoting Domains were generated and accorded the designations “DART B,” “DART C,” and “DART D.” One diabody having four chains and comprising CH1/CL Domains was generated and accorded the designation “DART E.” Two trivalent binding molecules having four chains and comprising E/K-coil Heterodimer-Promoting Domains and CH1/CL Domains were generated and accorded the designations “TRIDENT A,” and “TRIDENT B.”

In addition, several antibodies having specificity for PD-1 or CTLA-4 were generated. One antibody specific for PD-1 was generated and accorded the designation “PD-1 mAb 6 G4P.” Three antibodies specific for CTLA-4 were generated and accorded the designations “CTLA-4 mAb 1,” “CTLA-4 mAb 3 G1AA,” and “CTLA-4 mAb 3 G4P.”

The structure and amino acid sequences of these PD-1×CTLA-4 bispecific molecules, anti-PD-1 antibodies, anti-CTLA-4 antibodies are provided above and are summarized in Table 9 below.

TABLE 9

| | Variable | | | SEQ ID | Other |
|---|---|---|---|---|---|
| Name | Regions | Fc^{‡} | Chains | NOs: | Components |
| DART B | CTLA-4 mAb 1 | IgG4 | 1 | 95 | E/K-Coils; |
| | PD-1 mAb 6-ISQ | (YTE) | 2 | 96 | see FIG. 3B |
| | | | 3 | 95 | |
| | | | 4 | 96 | |
| DART C | CTLA-4 mAb 3 | IgG4 | 1 | 97 | E/K-Coils; |
| | PD-1 mAb 6-ISQ | | 2 | 98 | see FIG. 3B |
| | | | 3 | 97 | |
| | | | 4 | 98 | |
| DART D | PD-1 mAb 6-ISQ | IgG4 | 1 | 99 | E/K-Coils; |
| | CTLA-4 mAb 3 | (YTE) | 2 | 100 | see FIG. 3B |
| | | | 3 | 99 | |
| | | | 4 | 100 | |
| DART E | CTLA-4 mAb 3 | IgG4 | 1 | 102 | CL/CH1; |
| | PD-1 mAb 6-ISQ | (YTE) | 2 | 103 | see FIG. 3C |
| | | | 3 | 102 | |
| | | | 4 | 103 | |
| DART F | PD-1 mAb 6-ISQ | IgG1 | 1 | 101 | E/K-Coils; |
| | CTLA-4 mAb 3 | (AA/ | 2 | 100 | see FIG. 3B |
| | | YTE) | 3 | 101 | |
| | | | 4 | 100 | |
| TRIDENT | PD-1 mAb 6-ISQ | IgG4 | 1 | 104 | E/K-Coils |
| A | CTLA-4 mAb 3 | (YTE) | 2 | 105 | and CL/CH1; |
| | | | 3 | 106 | see FIG. 6A |
| | | | 4 | 107 | |
| TRIDENT | PD-1 mAb 6-ISQ | IgG1 | 1 | 108 | E/K-Coils |
| B | CTLA-4 mAb 3 | (AA/ | 2 | 105 | and CL/CH1; |
| | | YTE) | 3 | 109 | see FIG. 6A |
| | | | 4 | 107 | |
| PD-1 | PD-1 mAb 6-ISQ | IgG4 | 1 | 88 | natural |
| mAb 6 | | | 2 | 89 | antibody |
| G4P | | | 3 | 88 | structure |
| | | | 4 | 89 | |
| CTLA-4 | CTLA-4 mAb 1 | IgG1 | 4 | ** | natural |
| mAb 1 | (ipilimumab | | | | antibody |
| | replica) | | | | structure |
| CTLA-4 | CTLA-4 mAb 3 | IgG1 | 1 | 92 | natural |
| mAb 3 | | (AA) | 2 | 94 | antibody |
| G1AA | | | 3 | 92 | structure |
| | | | 4 | 94 | |
| CTLA-4 | CTLA-4 mAb 3 | IgG4 | 1 | 93 | natural |
| mAb 3 | | | 2 | 94 | antibody |
| G4P | | | 3 | 93 | structure |
| | | | 4 | 94 | |

^{‡}Molecules incorporating IgG4 Fc regions also incorporate a stabilized IgG4 hinge region.
** the same amino acid sequence as ipilimumab (see, e.g., IMGT 3D and 2D Structural Database Accession Nos. 8568_H and 8568_L)

Additional PD-1×CTLA-4 bispecific molecules comprising alternative PD-1 and/or CTLA-4 epitope-binding sites may be readily generated by incorporating different VH and VL Domains. Similarly, molecules comprising alternative linkers, Fc Regions, and/or having alternative structures may be generated as provided herein (see, e.g., Table 8).

A. ELISA Binding Studies

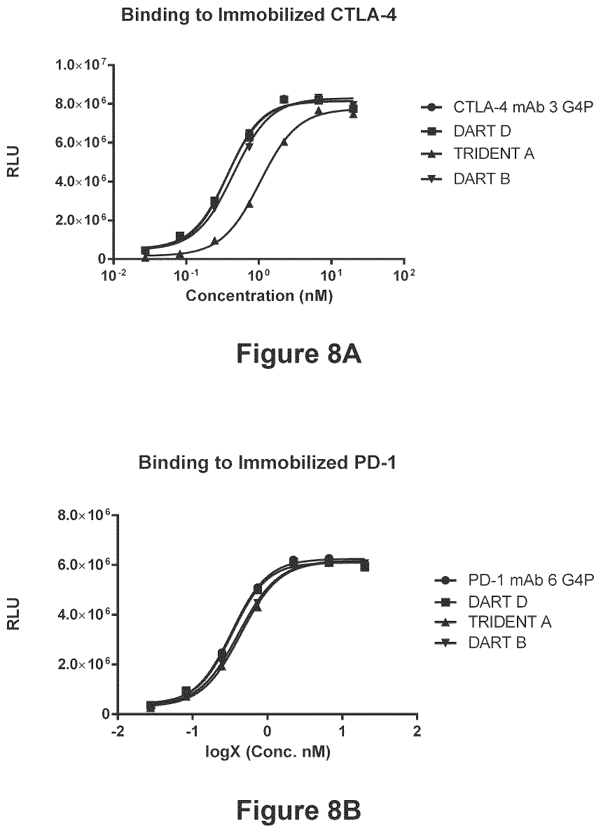

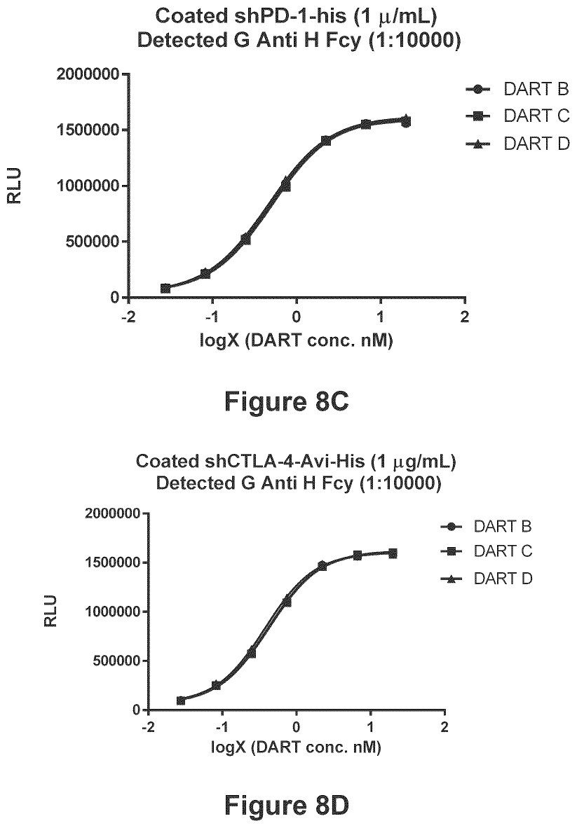

ELISA studies were conducted to measure the binding of serially diluted binding molecules (antibody CTLA-4 mAb 3 G4P, DART D, TRIDENT A or DARTB) to soluble hCTLA-4-Avi-His (1 μg/mL) or hPD-1-His (1 μg/mL) that had been coated onto support plates. Goat anti-human-Fc-HIRP (1:10,000) was employed as the secondary detection molecule to detect binding. The results of such studies are shown in Table 10 and in FIGS. 8A-8B. The data shows that PD-1×CTLA-4 bispecific molecules having two binding sites for PD-1 and CTLA-4 (e.g., DART D and DART B) exhibited binding to PD-1 and CTLA-4 that was similar to that of their respective parental anti-PD-1 and anti-CTLA-4 antibodies. PD-1×CTLA-4 bispecific molecules having two binding sites for PD-1 and one binding site for CTLA-4 (e.g., TRIDENT A) exhibited binding to PD-1 that was similar to that of the parental anti-PD-1 antibody and exhibited reduced binding to CTLA-4 (relative to that of the parental antibody) due to the reduced avidity of the trivalent molecule, which comprises only a single binding site for CTLA-4. Similar binding results were observed for DART F and TRIDENT B having IgG1 CH1 and/or IgG1 (AA/YTE) Fc regions.

TABLE 10

| | EC₅₀ of CTLA- | EC₅₀ of PD-1 |
|---|---|---|
| Construct | 4 Binding (nM) | Binding (nM) |
| CTLA-4 mAb 3 G4P | 0.4 | N/A |
| PD-1 mAb 6 G4P | N/A | 0.3 |
| DART D | 0.4 | 0.3 |
| TRIDENT A | 1.0 | 0.4 |
| DART B | 0.4 | 0.4 |

The effect of altering orientations and binding domains on binding was investigated by incubating PD-1×CTLA-4 bispecific molecules comprising the CTLA-4 binding domains of CTLA-4 mAb 1 (e.g., DART B) and CTLA-4 mAb 3 (e.g., DART C and DART D) in the presence of soluble human PD-1 (FIG. 8C), or soluble human CTLA-4-Avi-His (FIG. 8D), that had been coated onto support plates. Goat anti-human-Fcγ-HRP was employed as the secondary detection molecule to detect binding using PICO chemiluminescent substrate. The results indicate that PD-1×CTLA-4 bispecific molecules comprising the CTLA-4 binding domains of CTLA-4 mAb 1 (e.g., DART B) and CTLA-4 mAb 3 (e.g., DART C and DART D) exhibit similar binding to CTLA-4. The orientation of the binding domains (i.e., location on first or second chain) was not found to significantly alter binding to PD-1 or CTLA-4 (compare binding of DART C and DART D).

B. ELISA Blocking Studies

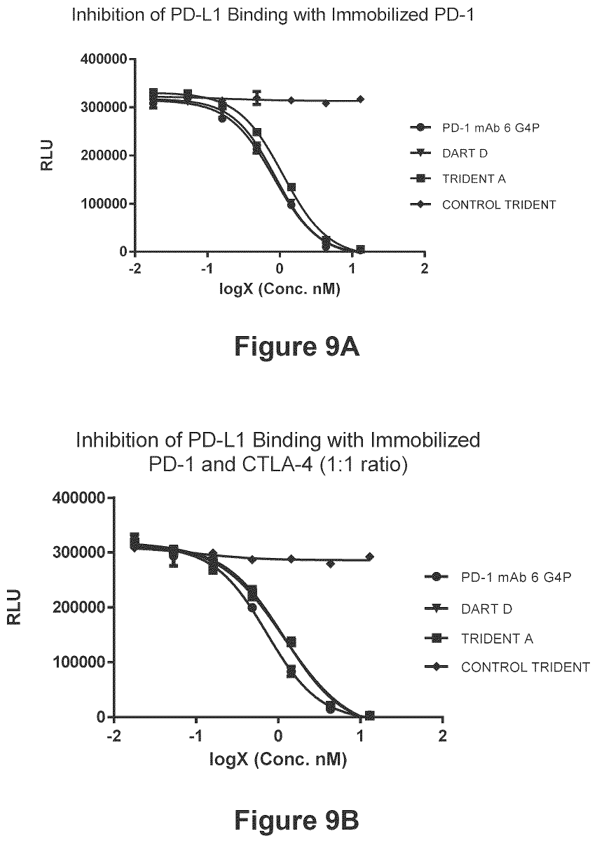

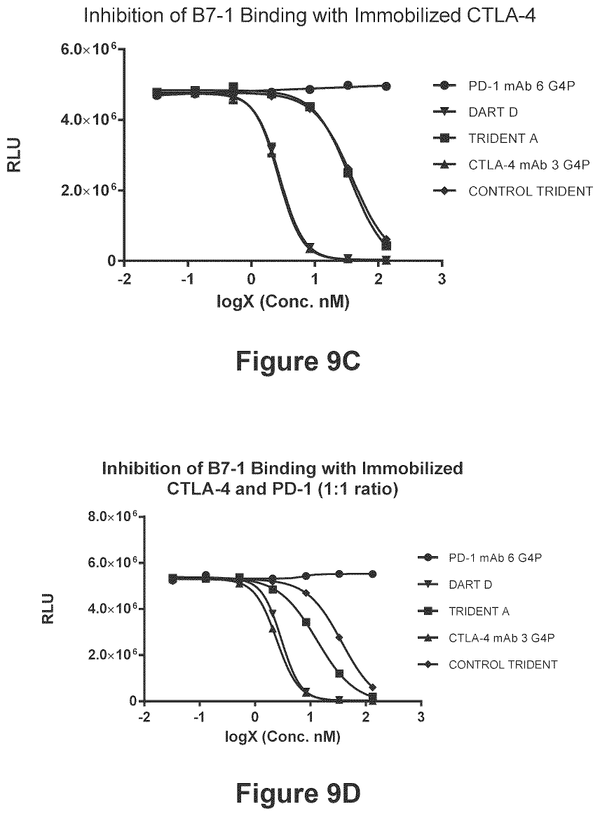

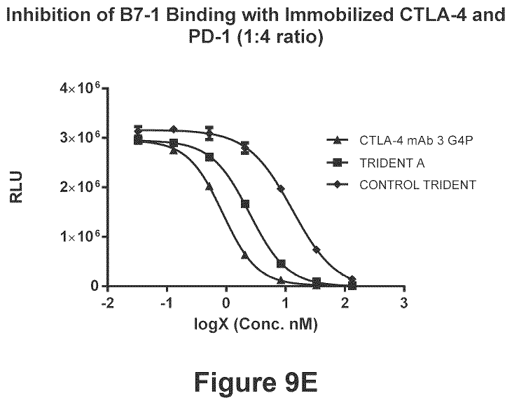

A series of ELISA assays were conducted to evaluate the ability of bispecific molecules of the invention to block ligand binding to PD-1 and CTLA-1, alone and in combination. Blockade of PD-L1 binding to PD-1 was evaluated in the presence of equal amounts of an irrelevant antigen and in the presence of equal amounts of CTLA-4. Plates were coated with a 11 mix of His-tagged soluble human PD-1 (shPD-1) and a His-tagged irrelevant antigen (irrAg) (2 μg/ml each), or a 11 mix of shPD-1 and a His-tagged soluble human CTLA-4 (shCTLA-4) (2 μg/ml each). PD-1 mAb 6 G4P, DART D, TRIDENT A or a CONTROL TRIDENT (having two binding sites for RSV and one binding site for CTLA-4) at the indicated concentrations were premixed for 5 mins with 6 μg/ml biotin-labeled PD-L1 and added to the plates. PD-L1 binding was detected using streptavidin HRP (1:3,000). The results of this evaluation are presented in FIGS. 9A-9B. All of the PD-1 binding molecules tested were found to be able to inhibit PD-L1 binding to PD-1.

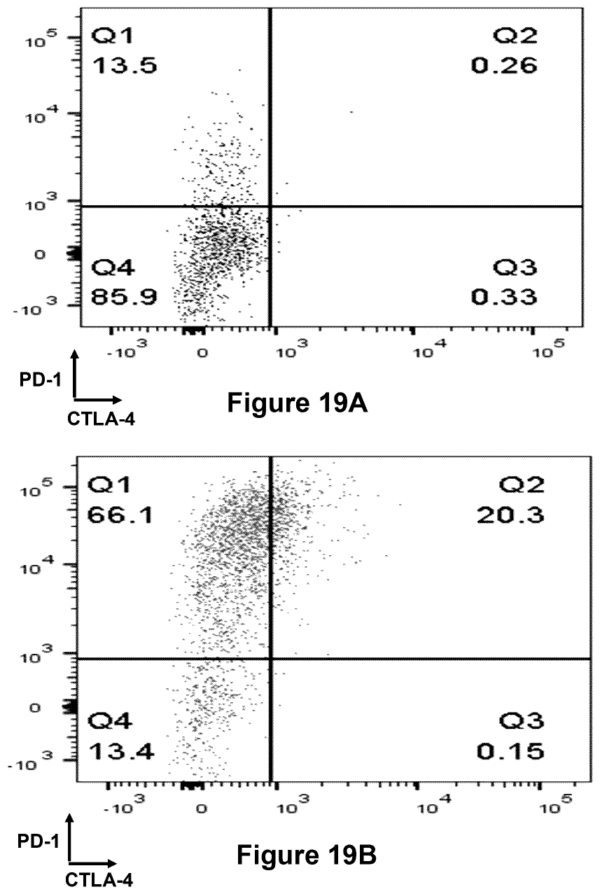

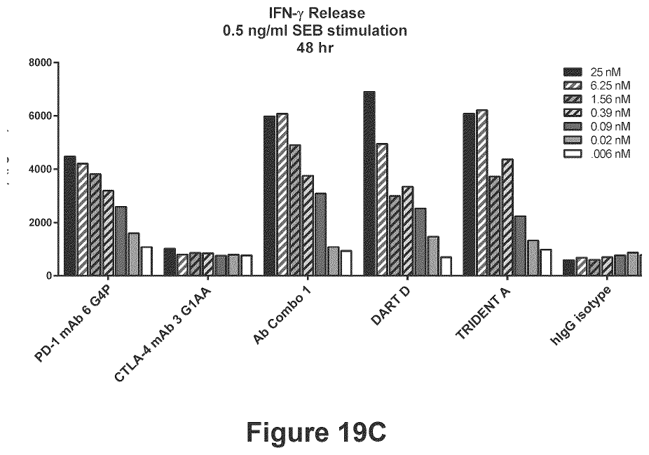

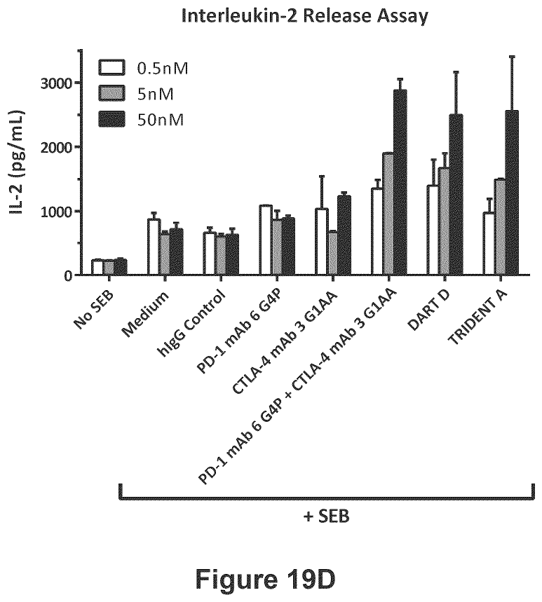

Blockade of B7-1 binding to CTLA-4 was evaluated in the presence of equal amounts of an irrelevant antigen and in the presence of equal amounts of, or four-fold more PD-1. Plates were coated with a 1:1 mix of shCTLA-4 and irrAg (2 μg/ml each), a 1:1 mix of shCTLA-4 shPD-1 (2 μg/ml each), or a 1:4 mix of shCTLA-4 (0.8 μg/ml) and shPD-1 (3.2 g/ml). PD-1 mAb 6 G4P, DART D, TRIDENT A, CTLA-4 mAb 3 G4P, or CONTROL TRIDENT at indicated concentrations were premixed for 5 mins with 0.2 μg/ml biotin-labeled B7-1 and added to the plates. B7-1 binding was detected using streptavidin HRP (1:3,000). The results of this evaluation are presented in FIG. 9C-9E. All of the CTLA-4 binding molecules tested were found to be able to inhibit B7-1 binding to CTLA-4. TRIDENT A blocking of B7-1 binding was found to be enhanced by the interaction of its PD-1 binding arm interacting with immobilized PD-1 (compare to CONTROL TRIDENT which does not bind PD-1) (FIG. 9D). Moreover, under the 1:4 CTLA-4:PD-1 condition, which better mimics the relative expression levels seen on stimulated cells (see, FIG. 19A), TRIDENT A blocking of B7-1 binding was found to be further enhanced (i.e., the TRIDENT A curve was further shifted compared to the curve of the CONTROL TRIDENT, which does not bind PD-1) (FIG. 9E).

The results of these ELISA studies demonstrate that all of the PD-1 binding molecules tested were able to inhibit PD-L1 from binding to the PD-1 (FIGS. 9A-9B). All such molecules are bivalent for PD-1 and exhibited similar inhibition profiles. All of the CTLA-4 binding molecules tested were able to inhibit B7-1 from binding to immobilized CTLA-4 (FIG. 9C-9E) with molecules comprising two PD-1 binding sites and one CTLA-4 binding site exhibiting stronger inhibition in the presence of PD-1 (FIG. 9D-9E). Thus, the trivalent molecules comprising a single CTLA-4 binding site exhibit a PD-1 biased blockade of CTLA-4 ligands, demonstrating that the CTLA-4 interaction can be tailored by adjusting the valency.

C. BIACORE® Studies

The binding affinity of DART A, TRIDENT A, and CTLA-4 mAb 1 to human CTLA-4 and cynomolgus monkey CTLA-4 was investigated using BIACORE® analysis. Briefly, His-tagged soluble CTLA-4 (an extracellular portion of human or cynomolgus monkey CTLA-4 fused to a histidine-containing peptide) was captured on immobilized anti-PentaHis and then different concentrations (12.5-200 nM) of the CTLA-4 binding molecules were passed over the immobilized CTLA-4 proteins. The kinetics of binding were determined via BIACORE® analysis (affinity by 1:1 Langmuir binding model (simultaneous ka/kd); or avidity by separate ka/kd 1:1 fit). The calculated kₐ, k_{d} and K_{D} from these studies are presented in Table 11.

TABLE 11

| | Human CTLA-4 | | | Cyno CTLA-4 | | |
|---|---|---|---|---|---|---|
| | kₐ | k_{d} | KD | kₐ | k_{d} | KD |
| Molecule | (×10⁵) | (×10⁻⁴) | (nM) | (×10⁵) | (×10⁻³) | (nM) |
| CTLA-4 mAb 1^{*} | 6.6 | 8.9 | 1.4 | 10 | 1.3 | 1.3 |
| DART D^{*} | 2.3 | 7.1 | 3.1 | 3.5 | 1.7 | 4.9 |
| TRIDENT A^{‡} | 1.2 | 32 | 26.7 | 2.5 | 65 | 260 |

^{*}avidity by separate ka/kd 1:1 fit
^{‡}affinity by 1:1 Langmuir binding model

DART D is bivalent for CTLA-4 and exhibits binding affinities to human and cynomolgus monkey CTLA-4 that are within about 2 to 4-fold that of the CTLA-4 mAb 1. TRIDENT A is monovalent for CTLA-4 exhibits lower affinity for both human and cynomolgus monkey CTLA-4 as expected in view of its reduced avidity.

The binding affinity of DART A, TRIDENT A, PD-1 mAb 6 G4P, and CTLA-4 mAb 3 G1AA to human PD-1 was investigated using BIACORE® analysis. The binding molecules were captured on immobilized F(ab)₂ goat anti-human Fc and then different concentrations (6.25-100 nM) of His-tagged soluble human PD-1 were passed over the immobilized binding molecules, and the kinetics of binding was determined via BIACORE® analysis (Langmuir 1:1 binding fit). The calculated ka, kd and KD from these studies are presented in Table 12 (n.d., not detectable).

TABLE 12

| | Human PD-1 | | |
|---|---|---|---|
| | kₐ | k_{d} | KD |
| Molecule | (×10⁵) | (×10⁻⁴) | (nM) |
| CTLA-4 mAb 3 G1AA | n.d. | n.d. | n.d. |
| PD-1 mAb 6 G4P | 6.2 | 6.7 | 1.1 |
| DART D | 4.8 | 8.1 | 1.7 |
| TRIDENT A | 5.2 | 6.8 | 1.3 |

DART A, TRIDENT A, PD-1 mAb 6 G4P are each bivalent for PD-1 and exhibit comparable binding affinities. As expected, CTLA-4 mAb 3 G1AA did not exhibit any detectable binding for human PD-1.

D. CTLA-4 Cell Based Assays

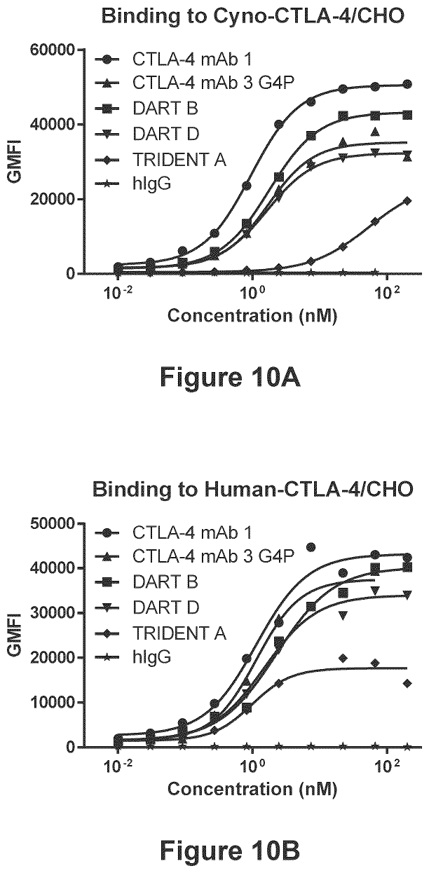

DART B, DART D, TRIDENT A, the anti-CTLA-4 antibodies CTLA-4 mAb 1, CTLA-4 mAb 3 G4P, and an hIgG control antibody were evaluated for binding to CHO cells expressing cynomolgus monkey CTLA-4 (cynoCTLA-4) or human CTLA-4 (huCTLA-4). The results of this evaluation are shown in FIGS. 10A-10B. The binding molecules were incubated in the presence of CHO cells that were expressing either cynomolgus monkey CTLA-4 (FIG. 10A) or human CTLA-4 (FIG. 10B). Binding to such cells was detected using an anti-human Fc secondary antibody. The results show that all the molecules tested were able to bind human and cynomolgus monkey CTLA-4 expressed on the surface of the CHO cells. The anti-CTLA-4 antibodies exhibited similar binding profiles to huCTLA-4; the bivalent, bispecific molecules DART B and DART D exhibited slightly reduced binding, and the trivalent binding molecule. TRIDENT A, which is monovalent for CTLA-4 exhibited lower binding than the molecules having higher valency for CTLA-4. The control antibody did not bind. Similar results were seen for binding to cynoCTLA-4.

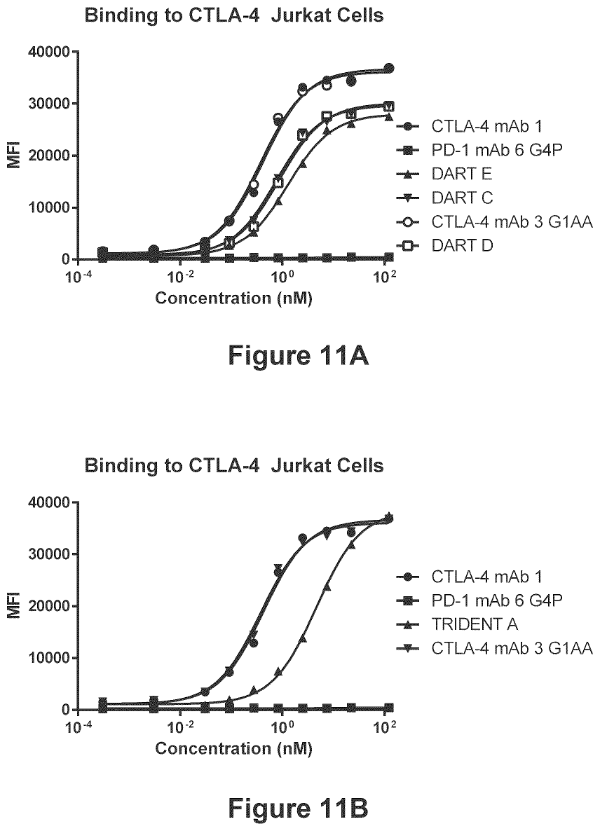

DART C, DART D, DART E, TRIDENT A, the anti-CTLA-4 antibodies CTLA-4 mAb 1, CTLA-4 mAb 3 G1AA, and the anti-PD-1 antibody PD-1 mAb 6 G4P were evaluated for binding to Jurkat cells which express huCTLA-4 but not PD-1 on their surface. Binding of the DART and TRIDENT molecules to human CTLA-4 was detected using anti-human FC secondary Ab (FACS). The results of the evaluation are shown in Table 13 and FIG. 11A (DART C, DART D, DART E, CTLA-4 mAb 1, CTLA-4 mAb 3 G1AA, and PD-1 mAb 6 G4P) and FIG. 11B (CTLA-4 mAb 1, CTLA-4 mAb 3 G1AA, PD-1 mAb 6 G4P and TRIDENT A). As shown in FIGS. 11A-11B, the PD-1 antibody did not bind CTLA-4, but all the CTLA-4 binding molecules tested were able to bind huCTLA-4 expressed on the surface of Jurkat cells. The anti-CTLA-4 antibodies exhibited similar binding profiles; the bivalent, bispecific molecules DART C, DART D, and DART E exhibited slightly reduced binding to Jurkat cells and the trivalent binding molecule. TRIDENT A, which is monovalent for CTLA-4 exhibited lower binding than the molecules having higher valency for CTLA-4.

TABLE 13

| Molecule | EC50 (nM) |
|---|---|
| CTLA-4 mAb 1 | 0.4215 |
| PD-1 mAb 6 G4P | 6.557 |
| CTLA-4 mAb 3 G1AA | 0.3728 |
| DART E | 1.269 |
| DART C | 0.7575 |
| DART D | 0.8829 |
| TRIDENT A | 4.638 |

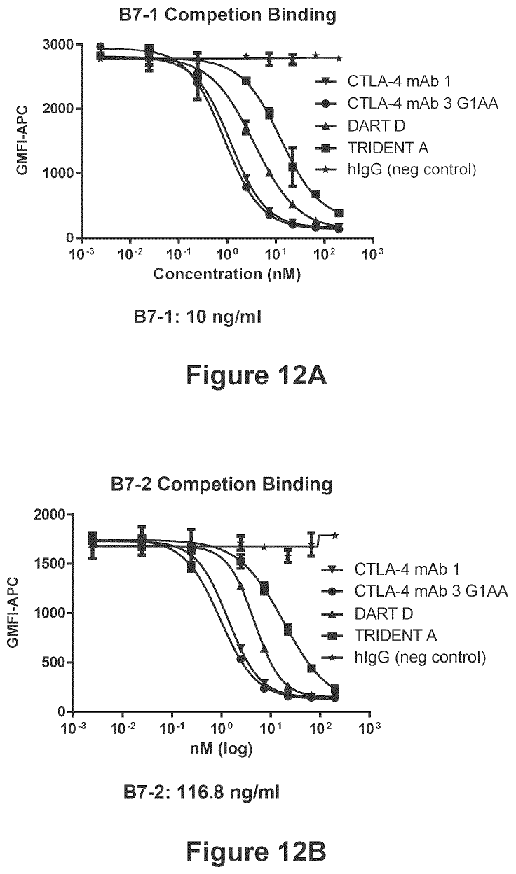

DART D, TRIDENT A and the anti-CTLA-4 antibodies CTLA-4 mAb 1, CTLA-4 mAb 3 G1AA were evaluated for their ability to block the CTLA-4 ligands B7-1 and B7-2. His-tagged derivatives of B7-1 and B7-2 were incubated in the presence of CTLA-4 Jurkat cells. Binding of His-B7-1 and His-B7-2 was detected using an anti-His antibody. The results of this evaluation are shown in FIG. 12A (His-B7-1) and FIG. 12B (His-B7-2). All the molecules tested were found to be able to inhibit B7-1 and B7-2 from binding CTLA-4 expressed on the surface of the Jurkat cells. The anti-CTLA-4 antibodies exhibited similar inhibition profiles; the bivalent, bispecific molecule DART D was slight less potent an inhibitor and the trivalent binding molecule. TRIDENT A, which is monovalent for CTLA-4 was less potent than any of the molecules having higher valency for CTLA-4. The control antibody did not inhibit at all. The ELISA studies described above suggest that TRIDENT A, and similar molecules having two PD-1 binding sites and one CTLA-4 binding site would be more potent inhibitors in the presence of PD-1.

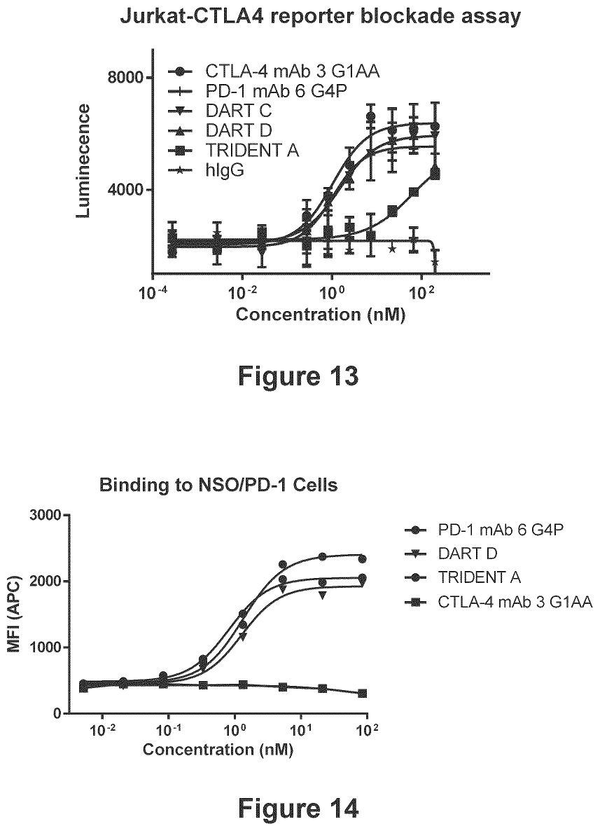

An IL-2/Luc Jurkat cell CTLA-4 reporter assay was used to evaluate the ability of DART C, DART D, TRIDENT A, CTLA-4 mAb 3 G1AA and PD-1 mAb 6 G4P to reverse CTLA-4 immune checkpoint inhibitory signal as demonstrated by increased luciferase expression. IL-2/Luc-Jurkat-CTLA-4 cells were therefore incubated in the presence of such molecules (R:S=1:0.3) for 30 min at 37° C., after which time artificial antigen presenting Raji cells were added and the incubation continued for 6 hours. The artificial antigen presenting cells activate the TCR/CD3 complex on the Jurkat reporter cells. The results of the evaluation are shown in FIG. 13. All of the CTLA-4 binding molecules tested were able reverse the CTLA-4 immune checkpoint inhibitory signal as determined by the luciferase assay. TRIDENT A, which is monovalent for CTLA-4 was less potent in this assay than any of the molecules having higher valency for CTLA-4. The control antibody did not inhibit at all. The ELISA studies described above suggest that TRIDENT A, and similar molecules having two PD-1 binding sites and one CTLA-4 binding site would be more potent in the presence of PD-1.

E. PD-1 Cell Based Assays

DART D, TRIDENT A, PD-1 mAb 6 G4P, and CTLA-4 mAb 3 G1AA were evaluated for their ability to bind NSO cells expressing PD-1 but not CTLA-4. Binding molecules were incubated in the presence of the cells and the mean fluorescence index of the cells was measured. The results of this evaluation are presented in FIG. 14. As expected, the CTLA-4 antibody did not bind, all the bispecific binding molecules were found to be able to bind PD-1 expressed on the surface of NSO cells. All the bispecific molecules are bivalent for PD-1 and exhibited similar binding to NSO cells.

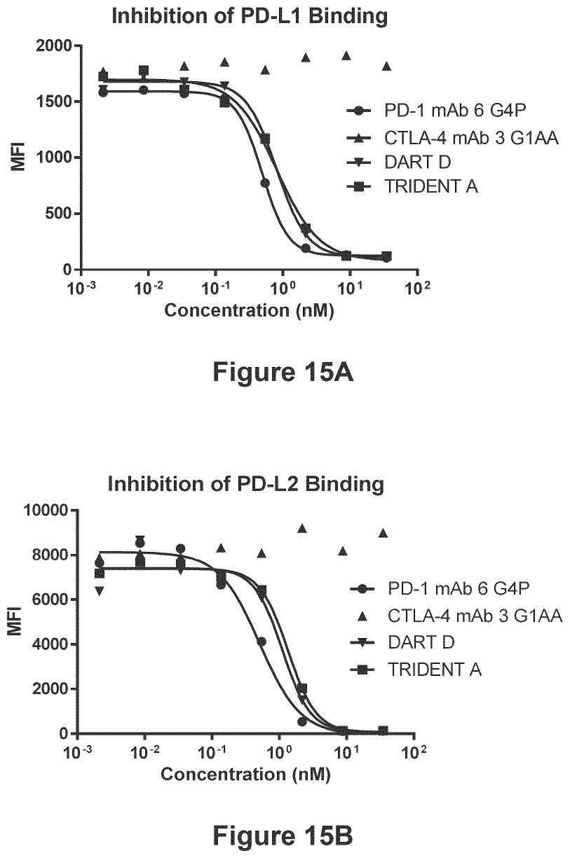

DART D, TRIDENT A, PD-1 mAb 6 G4P, and CTLA-4 mAb 3 G1AA were evaluated for their ability to block binding between PD-1 expressed on the cell surface and its ligands PD-L1 and PD-L2. PD-L1-PE or PD-L2-PE was incubated in the presence of such binding molecules and their ability to bind to NSO-PD-1 cells was evaluated using FACS. The results of this evaluation are presented in FIG. 15A (PD-L1) and FIG. 15B (PD-L2). As expected, the CTLA-4 antibody did not inhibit, all of the PD-1 binding molecules tested were able to inhibit both PD-L1 (FIG. 15A) and PD-L2 (FIG. 15B) from binding to the PD-1 expressed on the surface of the NSO cells. All the PD-1 binding molecules are bivalent for PD-1 and exhibited similar inhibition profiles.

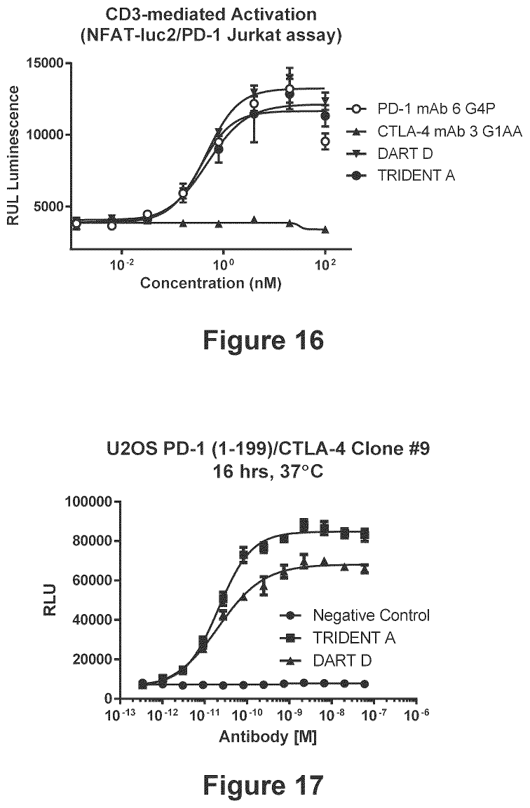

DART D, TRIDENT A, CTLA-4 mAb 3 G1AA, and PD-1 mAb 6 G4P were also evaluated in a PD-1 blockade reporter assay. Such binding molecules were incubated in the presence of PD-L1⁺ CHO and Jurkat effector cells, and the ability of the binding molecules to block immune inhibition (by blocking the PD-1/PD-L1 interaction) was assessed by following the extent of CD3-mediated activation (as demonstrated by increased luciferase expression in the NFAT-luc/PD-1 Jurkat assay; Promega). The results of this evaluation are presented in FIG. 16. All of the PD-1 binding molecules tested were able to reverse the PD-1 immune checkpoint inhibitory signal as demonstrated by increased luciferase expression. All the PD-1 binding molecules are bivalent for PD-1 and exhibited similar ability to inhibit PD-1 blockade of T cell signaling. The CTLA-4 antibody did not inhibit at all in this system.

F. CTLA-4/PD-1 Cell Based Assays

DART D, TRIDENT A, and a negative control antibody were examined for their ability to co-ligate PD-1 and CTLA-4 in an enzyme-fragment complementation assay by DiscoverX. In brief, aliquots of the U2OS CTLA-4(1-195)-PK PD-1(1-199)-EA cell line #9 were plated in quadruplicate at 5,000 cells/well in DiscoverX CP5 plating media on 384-well plates. Cells were allowed to attach for 4 hours at 37° C./5% CO₂. 11 point, 1:3 dilution series of each of the binding molecules were then added to the PD-1-CTLA-4 cells. The plates were incubated overnight (16 hrs) at 37° C./5% CO₂. PathHunter detection reagent was added to the wells, which were then incubated for 1 hour at room temperature in the dark, and the plate was then read on an Envision luminometer. The results of this evaluation are presented in Table 14 and FIG. 17 (U2OS CTLA-4(1-195)-PK PD-1(1-199)-EA cell line #9). Both the bispecific DART D and TRIDENT A molecules show comparable co-engagement of PD-1 and CTLA-4 in cells that co-express both receptors, as shown by enzyme-fragment complementation, indicating that the bispecific molecules of the invention are capable of simultaneous binding of PD-1 and CTLA-4, and further indicating that anchoring through PD-1 compensates for the decreased CTLA-4 avidity of the TRIDENT molecule when both target receptors are expressed. This finding is consistant with the ELISA inhibition studies described above. The negative control elicited no significant increase in signal in the PD1-CTLA4 cell line. Incubation with higher concentrations of TRIDENT A elicited a robust signal increase in the U2OS PD1-CTLA4 Dimerization cell line (S:B=12.7). The response with DART D in dose-response testing in the PD-1-CTLA-4 cell line was smaller in magnitude (S:B=9.2) but the EC50 values were similar for both these molecules (EC50=20 pM).

TABLE 14

| | Negative Control | TRIDENT A | DART D |
|---|---|---|---|
| HillSlope | ~15.99 | 1.103 | 0.8095 |
| EC50 (nM) | ~6.883 × 10⁻¹⁰ | 2.123 × 10⁻¹¹ | 2.090 × 10⁻¹¹ |

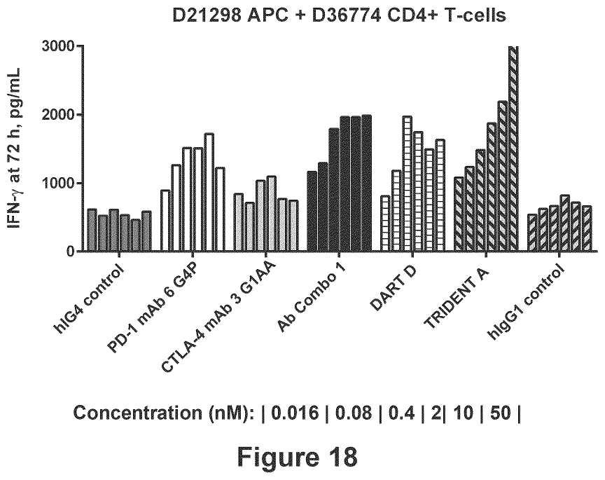

The ability of DART D, TRIDENT A, CTLA-4 mAb 3 G1AA, PD-1 mAb 6 G4P and the combinations of CTLA-4 mAb 3 G1AA/PD-1 mAb 6 G4P (Ab Combo 1) to enhance the response of a Mixed Lymphocyte Reaction (MLR) was evaluated. Monocyte-derived dendritic cells were generated by treating CD14+ monocytes (isolated from PBMCs using Miltenyi positive selection kit) with GM-CSF (100 ng/ml) and IL-4 (10 ng/ml) and then culturing the cells for 7 days. On day 7, cells were harvested and plated into 96-well plates and cultured for 24 h. On day 8, CD4+ T-cells (isolated by negative selection using Myltenyi kit) at 200,000 cells/well and test articles were added and cultured for 3 days. IFN-g levels in culture supernatants were then measured using using human DuoSet ELISA Kits for IFN-7 (R&D Systems) according to the manufacturer's instructions. When antibodies were used in combination, each antibody was added at the indicated concentration so that the total concentration of antibody added is doubled. The release of IFN-7 is plotted in FIG. 18. Both the bispecific DART D and TRIDENT A molecules were found to enhance the MLR response to the same extent or slightly better than the combination of individual parental antibodies.

The ability of DART D, TRIDENT A, CTLA-4 mAb 3 G1AA, PD-1 mAb 6 G4P and the combination of CTLA-4 mAb 1/PD-1 mAb 1 (Ab Combo 1) to enhance cytokine release through checkpoint inhibition was also evaluated in a Staphylococcus aureus enterotoxin type B (SEB) re-stimulation assay. In general, PBMCs were purified from whole blood (e.g., using the Ficoll-Paque Plus density gradient centrifugation method (GE Healthcare) according to manufacturer's instructions) from healthy donors. Purified PBMCs were cultured in RPMI-media+10% heat inactivated FBS+1% Penicillin/Streptomycin in T-25 bulk flasks for 2-3 days alone or with SEB (e.g., Sigma-Aldrich) at 0.5 ng/mL (primary stimulation). At the end of the first round of SEB-stimulation, PBMCs are washed twice with PBS and immediately plated in 96-well tissue culture plates at a concentration of 1-5×10⁵ cells/well in media alone, media with a control or a test article, media with SEB at 0.5 ng/mL (secondary stimulation) and no antibody, or media with SEB and a control IgG or a test article, and were cultured for an additional 2-3 days. At the end of the second stimulation, supernatants were harvested to measure cytokine secretion (e.g., using human DuoSet ELISA Kits for IFNγ, IL-2, TNFα, IL-10, and IL-4 (R&D Systems) according to the manufacturer's instructions).

FIGS. 19A-19B show fluorescence-activated cell sorting (FACS) dot plots of the expression of PD-1 vs. CTLA-1 by such PBMCs in the absence (FIG. 19A) or presence (FIG. 19B) of SEB stimulation. FIG. 19C shows the effect of the SEB stimulation on IFN-7 secretion. PBMCs were stimulated with Staphylococcus aureus enterotoxin type B (SEB) at 0.5 ng/ml for 48 hours. Cells were then harvested, washed and re-plated in 96 well plates with antibodies at various concentrations with fresh SEB for an additional 48 hours. The supernatant was then harvested and analyzed by flow cytometry ELISA for IFN-7 production. Both the bispecific DART and the TRIDENT protein showed an increase in IFN-7 response that recapitulated the response observed with the combination of the individual parental mAbs. Similar results were seen in a SEB Stimulation assay in which the PBMCs were cultured with a high concentration (500 ng/mL) of SEB for 72 hours. To further investigate the affect of PD1×CTLA-4 bispecific molecules on the T-cell response, PBMCs were stimulated with 0.5 ng/ml SEB for 48 hours, harvested, washed and re-plated in 96-well plates with fresh SEB and either DART D, TRIDENT A, CTLA-4 mAb 3 G1AA, PD-1 mAb 6 G4P or the combination of CTLA-4 mAb 3 G1AA/PD-1 mAb 6 G4P (Ab Combo 1) for an additional 48 hours, and the released TL-2 was measured (FIG. 19D). FIGS. 19A-19D show that the administration of PD1×CTLA-4 bispecific molecules significantly enhanced T-cell responses. When antibodies were used in combination, each antibody was added at the indicated concentration so that the total concentration of antibody added is doubled.

### Example 6

### In Vivo Studies

A. Activity of a PD-1×CTLA-4 Bispecific Molecule in GVHD Murine Model

The activity of a representative PD1×CTLA-4 bispecific bivalent molecule, DART D was assessed in a PBMC implanted NOG murine model of Graft Versus Host Disease (GVHD). The study design is presented in Table 15.

TABLE 15

| | | | Dose | Route/ | |
|---|---|---|---|---|---|
| Group | N/sex | Treatment | (μg/kg) | Schedule | Cell Implant(s) |
| 1. | 7/F | DART D | 500 | IV/Q7D × 7 | PBMC (IP, 1E7) |
| 2. | 7/F | DART D | 50 | IV/Q7D × 7 | PBMC (IP, 1E7) |
| 3. | 7/F | DART D | 5 | IV/Q7D × 7 | PBMC (IP, 1E7) |
| 4. | 7/F | Vehicle | 0 | IV/Q7D × 7 | PBMC (IP, 1E7) |

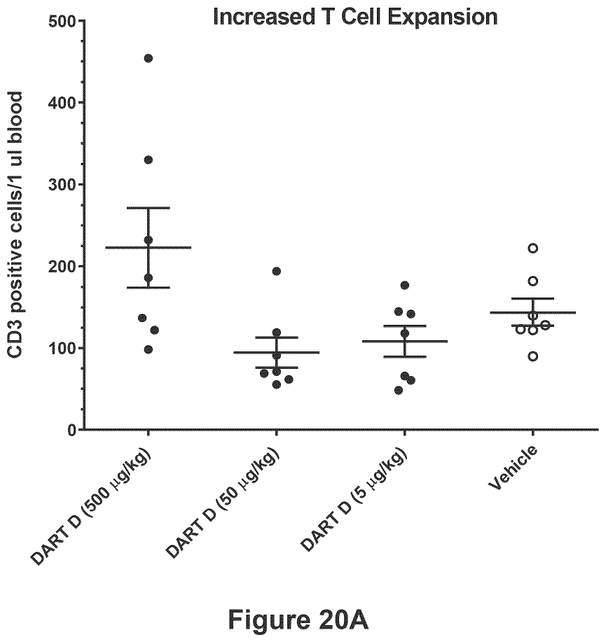

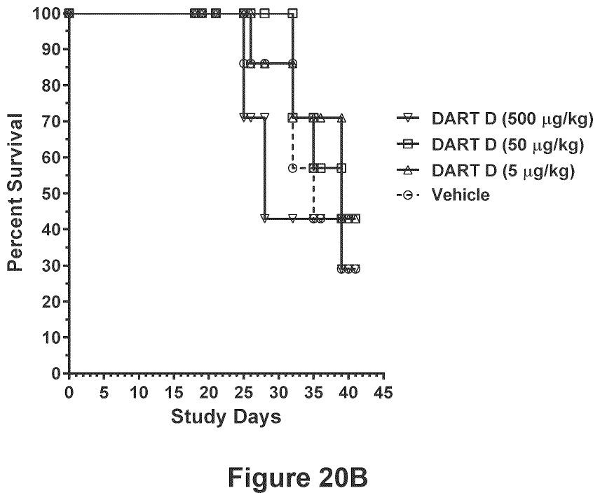

CD3+ T cell counts were performed via FACS on study day 14 and are plotted in FIG. 20A. Survival was monitored over the course of the study and is plotted as percent survival in FIG. 20B. Increased T cell expansion and accelerated GVHD was seen in animal treated with 500 μg/kg DART D, consistence with enhancement of T cell immune responses.

B. Toxicology and Pharmacokinetic Study of PD-1×CTLA-4 Bispecific Molecules

The safety profile of a representative PD1×CTLA-4 bispecific bivalent molecule, DART D, and a representative PD1×CTLA-4 bispecific trivalent molecule, TRIDENT A, was assessed in a non-GLP (Good Laboratory Practice) dosing study in cynomolgus monkeys. In addition, several markers pharmacodynamics activity were examined.

In this study the potential toxicity of the PD-1×CTLA-4 bispecific molecules, when administered by multiple intravenous infusions was evaluated. The study design is presented in Table 16.

TABLE 16

| | | | | Number of |
|---|---|---|---|---|
| Group | Test Article | Dose (mg/kg) | Dose Days | Animals |
| 1 | Control | 0 | 1, 8, 15 | 1M 1F |
| 2 | DART D | 50 | 1, 8, 15 | 3M 3F |
| 3 | DART D | 75 | 15, 22, 29 | 3M 3F |
| 4 | TRIDENT A | 5 | 1 | 2M 1F |

A 2-week interval was thus provided between the 50 mg/kg dose and escalation to 75 mg/kg. The following parameters and endpoints were evaluated in this study: clinical signs, body weights, food consumption, body temperature, clinical pathology parameters (coagulation, clinical chemistry and hematology pre-dose and 23 hours post-dose for Groups 1-3; out to day 22 for Group 4), bioanalysis and toxicokinetic parameters, flow cytometry (pre-dose and 23 hours post dose), cytokines (2, 6, 22 hours post-dose). Anti-Drug-Antibodies were evaluated for Group 4 only on days 8, 15 and 22. Necropsy was performed 48 hours after the 3^{rd} dose for Groups 1-3 only. The in vivo binding and activity of the PD-1×CTLA-4 bispecific molecules was also examined as described below.

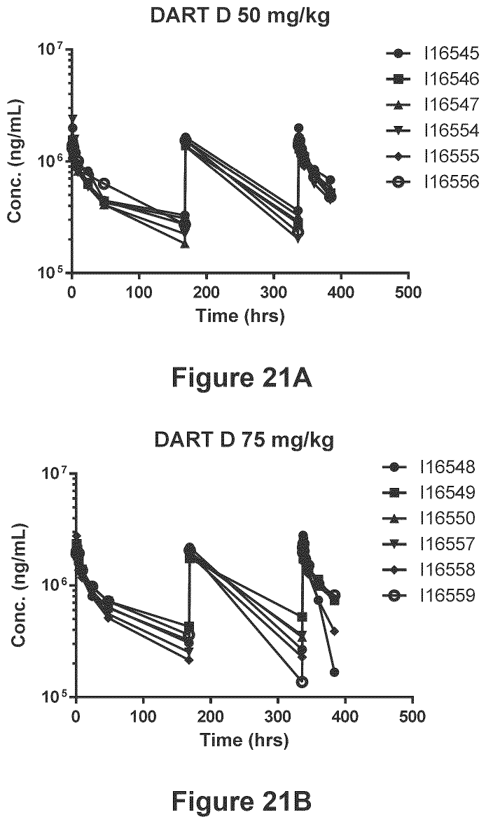

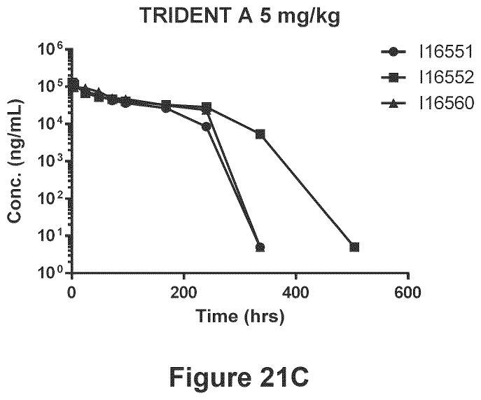

All animals survived until scheduled euthanasia. No adverse clinical observations in animals receiving 3 doses up to 75 mg/kg/week. In particular, no diarrhea was observed. The histopathology was also unremarkable. Increases in globulin levels were observed in the treatment groups and the organ weight of the spleen and thymus were observed to increase in Groups 2-3 (see Table 17, Group 4 was not necropsied), as would be expected upon stimulation of the immune system. The serum concentration-time profiles for each of the treatment groups are shown in FIGS. 21A-21C and are consistent with molecules comprising human Fc regions in cynomolgus monkeys.

TABLE 17

| | Test | Dose | Spleen: | Thymus: |
|---|---|---|---|---|
| Group | Article | (mg/kg) | Body Weight | Body Weight |
| 1 | Control | 0 | 0.080 (mean, n = 2) | 0.035 (mean, n = 2) |
| 2 | DART D | 50 | 0.239 (mean, n = 6) | 0.088 (mean, n = 6) |
| 3 | DART D | 75 | 0.225 (mean, n = 6) | 0.084 (mean, n = 6) |

It has been reported that increases in absolute lymphocyte count (ALC) after treatment with the anti-CTLA-4 antibody ipilimumab appear to correlate with clinical benefit and overall survival (see, e.g., Ku, G. Y., et al. (2010) “Single-Institution Experience With Ipilimumab In Advanced Melanoma Patients In The Compassionate Use Setting: Lymphocyte Count After 2 Doses Correlates With Survival” Cancer 116(7):1767-1775) indicating that ALC may be a useful pharmacodynamic (PD) endpoint. The ALC counts were examined in each of the above-described groups pre-treatment and post-treatment on days 2, 8, 9, 15 and 16. Occupancy of DART D or TRIDENT A binding sites on PD-1+ T cells was determined by measuring the mean fluorescent intensity (MFI) of anti-human IgG4 Alexa 488+ events in the CD4+/PD-1+ and CD8+/PD-1+ T cell populations under two conditions for each monkey blood sample. Under one condition, the MFI values obtained in the presence of excess DART D or TRIDENT A were used to determine the maximal DART D or TRIDENT A binding intensity on PD-1+ cells within each cell population. Under the second condition, the MFI values obtained in the presence of excess negative control were used to determine the binding intensity of PD-1+ cells within each cell population exhibited in the DART D or TRIDENT A-treated animal at the time of sample collection. The difference between the two conditions was used to calculate % occupancy of DART D or TRIDENT A binding sites on PD-1+ T cell subsets in DART D or TRIDENT A-treated animals as follows:

$$\begin{matrix}\% Occupancy of DART D or TRIDENT A \\ Binding Sites On PD ‐ 1 + T Cell Subsets\end{matrix} = \\ [ \text{} \frac{( \begin{matrix}MFI of Anti ‐ HuIg G 4 + Events in \\ the Presence of Excess AEX 1367\end{matrix} )}{( \begin{matrix}MFI of Anti ‐ HuIg G 4 + Events in the \\ Presence of Excess DART D or TRIDENT A\end{matrix} )} ] \times 100$$

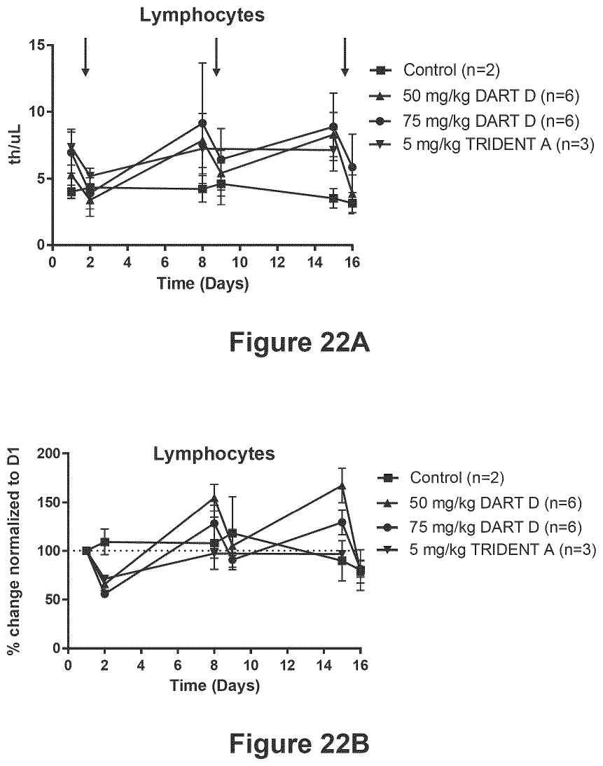

The absolute counts, and the percent change normalized to Day 1 are plotted in FIG. 22A (in thousands of cells/μl (th/μl)) and in FIG. 22B (percent change in the ALC normalized to Day 1 (D1)). Each of the DART D treatment groups exhibited an initial drop in ALC counts immediately after treatment followed by an increase in ALC to levels well above baseline. A similar trend was observed for the TRIDENT A treatment group, which only received only one lower dose.

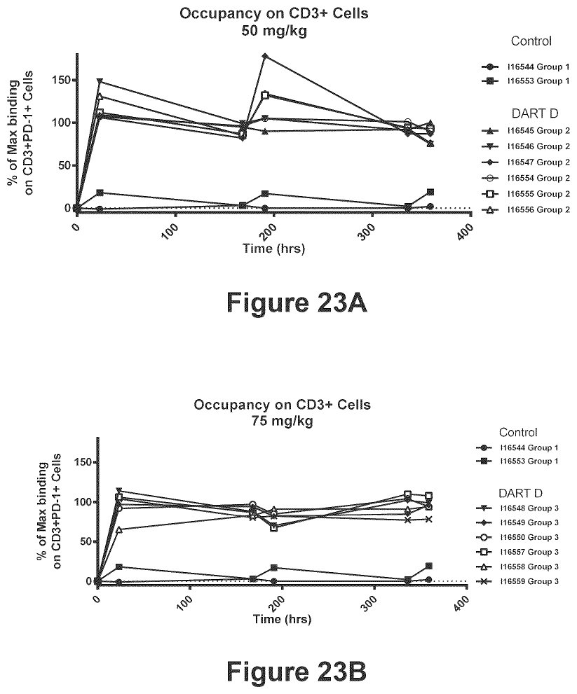

In addition, CD4+ T cell proliferation and PD-1 occupancy on T cells were examined for the above-described Groups 1-3. Briefly, CD3+/PD-1+ T cells were analyzed by FACS to evaluate the percent cells bound by DART D. Forty microliters of the negative control molecule (respiratory syncytial virus (RSV)×fluorescein IgG4,κ Fc DART) or test article (DART D or TRIDENT A) at 35 μg/mL were added to a 96 deep-well plate. One hundred microliters of well-mixed anticoagulated whole blood were then added into each well, thoroughly mixed using a pipette, and incubated in the dark for 45 to 75 minutes at ambient temperature. One thousand microliters of 1×BD FACS Lysing solution were then added to each well and mixed using a pipette; the plate was then incubated in the dark for an additional 10 to 20 minutes at ambient temperature. The plate was then centrifuged at 400×g for 5 minutes and the supernatant was discarded. One thousand microliters of FACS buffer were added in each well and mixed as a washing step. The plate was then centrifuged at 400×g for 5 minutes and the supernatant was discarded. The cell pellet was resuspended with twenty microliters of Panel 1 antibody mix and incubated for 30 to 60 minutes at ambient temperature. The plate was washed as in previous wash steps. At the end of incubation, the plate was washed again and the cell pellet was finally resuspended in three-hundred microliters of FACS buffer and the samples were analyzed with a BD FACSCanto II cell analyzer. The results of the analysis are shown in FIGS. 23A-23B.

As shown in FIG. 23A (for DART D administered at 50 mg/kg) and FIG. 23B (for DART D administered at 75 mg/kg), PD-1 occupancy (i.e., binding by DART D) was maximal throughout the duration of treatment for Groups 2 and 3. Proliferation CD4+ T cells were evaluated by FACS for co-expression of Ki-67 (a cellular marker for proliferation).

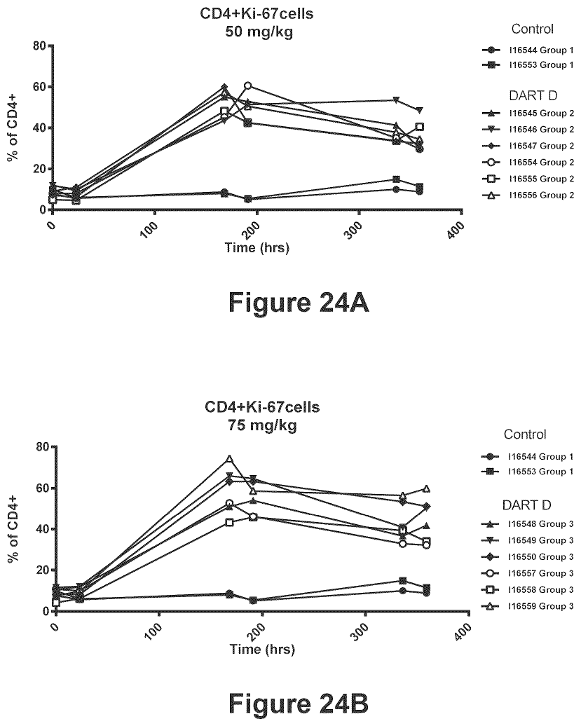

Twenty microliters of an antibody mixture A (containing antibodies that bind cell surface markers: CD45, CD3, CD4, and CD8) were added into a 96 deep-well plate. Fifty microliters of well-mixed anticoagulated whole blood were then added into each well, mixed thoroughly using a pipette, and incubated in the dark for 15 to 45 minutes at ambient temperature. Five hundred microliters of 1×BD FACS Lysing solution were then added to each well and mixed using a pipette; the plate was then incubated in the dark for an additional 10 to 20 minutes at ambient temperature. The plate was centrifuged at 1200 rpm for 5 minutes and the supernatant was discarded. Five hundred microliters of FACS buffer were then added in each well and mixed as a washing step. The plate was then centrifuged at 1200 rpm for 5 minutes and the supernatant was discarded. The cell pellet was resuspend in antibody mixture B (containing antibodies that bind the intracellular marker, Ki 67) or were resuspended in an iso antibody preparation (containing isotype controls for the intracellular marker) and incubated in the dark for 15 to 45 minutes. After washing, the cell pellet was resuspended in three hundred microliters of FACS buffer and the samples were analyzed with a BD FACSCanto II cell analyzer. From a T Cell Intracellular Staining Panel, the percentage of CD4+ and CD8+ cells was determined as the fraction of total CD45+ leukocyte gated cells. The cellular events of Ki 67+ in gated CD4+ cells were counted and the percentage of CD4+/Ki 67+ T cells (proliferative CD4 T cells) was determined as the fraction of total CD4+ cells. In a similar manner, the percentage of CD8+/Ki 67+ T cells (proliferative CD8 T cells) was determined as the fraction of total CD8+ cells. The results of the analysis are shown in FIGS. 24A-24B.

As shown in FIGS. 24A-24B, proliferation of CD4+ T cells was markedly enhanced in treatment Groups 2 and 3 throughout the duration of treatment. The results of this study indicate that administration of PD1×CTLA-4 bispecific molecules is well tolerated in cynomolgus monkeys at concentrations of up to 75 mg/kg. Well above the 5 mg/kg dosage where adverse events have been reported for cynomolgus monkeys treated with Ipilimumab. The molecules exhibited a favorable pharmacokinetic profile and a number of markers pharmacodynamics activity were observed including increased lymphocyte count, increased globulin levels, increased spleen and thymus organ weights, increased T cell proliferation (both T cell counts and expression of Ki-67) and maximal PD-1 occupancy on T cells.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for treating a disease or condition associated with a suppressed immune system, comprising administering to a subject in need thereof a composition comprising a bispecific molecule in an amount effective to treat the disease or condition, wherein the bispecific molecule comprises two polypeptide chains each comprising the polypeptide of SEQ ID NO:99 and two polypeptide chains each comprising the polypeptide of SEQ ID NO:100.

2. The method of claim 1, wherein the disease or condition is a cancer or an infection.

3. The method of claim 2, wherein the cancer is characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumor, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, a hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

4. The method of claim 2, wherein the infection is characterized by the presence of a bacterial, fungal, viral or protozoan pathogen.

5. A method for stimulating an immune-mediated response, comprising administering to a subject in need thereof a composition comprising a bispecific molecule in an amount effective to stimulate an immune-mediated response, wherein the bispecific molecule comprises two polypeptide chains each comprising the polypeptide of SEQ ID NO:99 and two polypeptide chains each comprising the polypeptide of SEQ ID NO:100.

6. A method for treating a disease or condition associated with a suppressed immune system, comprising administering to a subject in need thereof a composition comprising a bispecific molecule in an amount effective to treat the disease or condition, wherein the bispecific molecule comprises two polypeptide chains each comprising the polypeptide of SEQ ID NO:101 and two polypeptide chains each comprising the polypeptide of SEQ ID NO:100.

7. The method of claim 6, wherein the disease or condition is a cancer or an infection.

8. The method of claim 7, wherein the cancer is characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumor, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, a hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

9. The method of claim 7, wherein the infection is characterized by the presence of a bacterial, fungal, viral or protozoan pathogen.

10. A method for stimulating an immune-mediated response, comprising administering to a subject in need thereof a composition comprising a bispecific molecule in an amount effective to stimulate an immune-mediated response, wherein the bispecific molecule comprises two polypeptide chains each comprising the polypeptide of SEQ ID NO:101 and two polypeptide chains each comprising the polypeptide of SEQ ID NO:100.

11. A method for treating a disease or condition associated with a suppressed immune system, comprising administering to a subject in need thereof a composition comprising a bispecific molecule in an amount effective to treat the disease or condition, wherein the bispecific molecule comprises one polypeptide chain comprising the polypeptide of SEQ ID NO:104, one polypeptide chain comprising the polypeptide of SEQ ID NO:105, one polypeptide chain comprising the polypeptide of SEQ ID NO:106, and one polypeptide chain comprising the polypeptide of SEQ ID NO:107.

12. The method of claim 11, wherein the disease or condition is a cancer or an infection.

13. The method of claim 12, wherein the cancer is characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumor, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, a hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

14. The method of claim 12, wherein the infection is characterized by the presence of a bacterial, fungal, viral or protozoan pathogen.

15. A method for stimulating an immune-mediated response, comprising administering to a subject in need thereof a composition comprising a bispecific molecule in an amount effective to stimulate an immune-mediated response, wherein the bispecific molecule comprises one polypeptide chain comprising the polypeptide of SEQ ID NO:104, one polypeptide chain comprising the polypeptide of SEQ ID NO:105, one polypeptide chain comprising the polypeptide of SEQ ID NO:106, and one polypeptide chain comprising the polypeptide of SEQ ID NO:107.

16. A method for treating a disease or condition associated with a suppressed immune system, comprising administering to a subject in need thereof a composition comprising a bispecific molecule in an amount effective to treat the disease or condition, wherein the bispecific molecule comprises one polypeptide chain comprising the polypeptide of SEQ ID NO:108, one polypeptide chain comprising the polypeptide of SEQ ID NO:105, one polypeptide chain comprising the polypeptide of SEQ ID NO:109, and one polypeptide chain comprising the polypeptide of SEQ ID NO:107.

17. The method of claim 16, wherein the disease or condition is a cancer or an infection.

18. The method of claim 17, wherein the cancer is characterized by the presence of a cancer cell selected from the group consisting of a cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumor, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, a hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

19. The method of claim 17, wherein the infection is characterized by the presence of a bacterial, fungal, viral or protozoan pathogen.

20. A method for stimulating an immune-mediated response, comprising administering to a subject in need thereof a composition comprising a bispecific molecule in an amount effective to stimulate an immune-mediated response, wherein the bispecific molecule comprises one polypeptide chain comprising the polypeptide of SEQ ID NO:108, one polypeptide chain comprising the polypeptide of SEQ ID NO:105, one polypeptide chain comprising the polypeptide of SEQ ID NO:109, and one polypeptide chain comprising the polypeptide of SEQ ID NO:107.
